(19) United States
(12) Patent Application Publication
(10) Pub. No.: US 2022/0288028 A1
(43) Pub. Date: Sep. 15, 2022

(54) A THERAPEUTIC APPROACH FOR TREATING NON-INFECTIOUS OCULAR IMMUNOINFLAMMATORY DISORDERS

(71) Applicant: The Schepens Eye Research Institute, Inc., Boston, MA (US)

(72) Inventors: Reza Dana, Newton, MA (US); Yihe Chen, Somerville, MA (US)

(21) Appl. No.: 17/613,467

(22) PCT Filed: May 29, 2020
(86) PCT No.: PCT/US2020/035222

§ 371 Date: Nov. 22, 2021

Related U.S. Application Data
(60) Provisional application No. 62/879,839, filed on Jul. 29, 2019.
(60) Provisional application No. 62/854,575, filed on May 30, 2019.

(51) Int. Cl. A61K 31/41; A61K 31/4035; A61P 27/02; A61K 31/404; A61K 31/439; A61K 31/451; A61K 31/5377; A61K 45/06; A61K 9/00

(52) U.S. Cl. CPC A61K 31/41; A61K 31/4035; A61P 27/02; A61K 31/404; A61K 31/439; A61K 31/451; A61K 31/5377; A61K 45/06; A61K 9/0048

(57) ABSTRACT

Disclosed are methods of treating a non-infectious ocular immunoinflammatory disorder in a subject. Methods of reducing a symptom, e.g., ocular redness, of a non-infectious ocular immunoinflammatory disorder in a subject, and pharmaceutical composition containing an SP blocker, an SP antagonist, an SP receptor blocker or an SP receptor antagonist as an active component and a pharmaceutically acceptable carrier or excipient are also described.

### CROSS REFERENCE TO RELATED APPLICATIONS

This Application claims the benefit of U.S. Provisional Application 62/879,839 filed on Jul. 29, 2019 and U.S. Provisional Application 62/854,575 filed on May 30, 2019. The entire contents of these applications are incorporated herein by reference in their entirety.

### FEDERALLY SPONSORED RESEARCH AND DEVELOPMENT

This invention was made with government support under R01-EY20889 awarded by the National Institutes of Health. The Government has certain rights in the invention.

### FIELD OF THE INVENTION

The present invention relates generally to the field of ophthalmology.

### BACKGROUND

Dry Eye Disease (DED) is characterized by chronic ocular surface inflammation, and is the most frequent non-refractive reason leading patients to seek professional eye care. It affects approximately 5 million Americans over the age of 50 years, with millions more experiencing intermittent symptoms of dry eye. The disease has an adverse effect on vision-related quality of life and productivity, and has caused a considerate economic burden on public health.

Ocular redness is one of the most commonly seen signs in ophthalmological clinic and is commonly due to the dilation of conjunctival blood vessels with infectious and non-infectious causes. Among the non-infectious ocular redness, DED and allergy are the two typical underlying disorders. Treatment of ocular redness depends on the underlying cause. The therapeutic strategies have been restricted to symptomatic relief

### SUMMARY

There remains an unmet need for treatment of DED and ocular redness.

Embodiments of the invention are directed to compositions and the use of those compositions in the treatment of DED, keratoneuralgia and/or ocular redness, e.g., ocular redness associated with a non-infectious ocular immunoinflammatory disorder. The present disclosure describes achieving immune quiescence by restoring or enhancing suppressive functions of regulatory T cells (Tregs) in non-infectious ocular immunoinflammatory disorders such as ocular redness, dry eye disease, and/or ocular pain, via blocking substance (SP) signaling. The endogenous receptor for substance P is neurokinin 1 receptor (NK1-receptor, NK1R). The results described herein, demonstrated that inhibition of SP-NK1R signaling restored or enhanced Treg functions, and thus suppresses inflammation and achieves immune quiescence in ocular immunoinflammatory disorders. NK1R antagonist confer clinical benefits to subject diagnosed with or suffering from the aforementioned Treg-associated ocular disorders.

Accordingly, in certain embodiments, a method of treating a non-infectious ocular immunoinflammatory disorder in a subject, comprises administering to a subject with a regulatory T cell (Treg)-associated ocular disorder, e.g., those described above, a composition comprising a therapeutically effective amount of a neurokinin 1 receptor (NK1R) antagonist wherein the NK1R antagonist increases or restores the Treg function as compared to a control.

In this and other embodiments, a Treg-associated ocular disorder is one selected from Dry Eye Disease (DED), non-DED-related ocular redness, allergic conjunctivitis and ocular pain.

In certain embodiments, the non-DED-related ocular redness is allergic ocular redness. In certain embodiments, the non-DED-related ocular redness is non-allergic ocular redness.

In conain orabodiraesus, a method of modulating regulatory T (Treg) cell activity or function comprises administering to a subject in need thereof, a pharmaceutical composition comprising a therapeutically effective amount of a substance P (SP) blocker, an SP antagonist, an SP receptor blocker, an SP receptor antagonist or combinations thereof as an active component. In certain embodiments, the SP signaling blockade-inducing agent is selected from an SP blocker, an SP antagonist, an SP receptor blocker and an SP receptor antagonist. In certain embodiments, the SP receptor is NK1R (SEQ ID NO:1).

Subjects suffering from an ocular immune inflammatory disease, e.g., Treg-associated ocular disorder, may suffer from one or more symptoms. In certain embodiments, a method of reducing a symptom of a non-infectious ocular immunoinflammatory disorder in a subject, comprises administering to the subject with a Treg-associated ocular disorder a composition comprising a therapeutically effective amount of an SP signaling blockade-inducing agent. In certain embodiments, the Treg-associated ocular disorder is one selected from Dry Eye Disease (DED), non-DED-related ocular redness, allergic conjunctivitis and ocular pain. Non-limiting examples of such symptoms include a sandy or gritty feeling (e.g., self-reported by a subject) as if something is in the eye, eye dryness, heavy eyelids, stinging, itching, burning, irritation, pain, redness, inflammation, discharge, inability to cry when emotionally stressed, eye fatigue, blurred vision, or excessive watering, and wherein the method inhibits or reduces the severity of the sandy or gritty feeling as if something is in the eye, eye dryness, heavy eyelids, stinging, itching, burning, irritation, pain, redness, inflammation, discharge, inability to cry when emotionally stressed, eye fatigue, blurred vision, and excessive watering. In embodiments, the method described herein may include identifying a subject having one or more of these symptoms.

Various implementations of the subject matter herein relate to the treatment of an ocular immunoinflammatory disorder that is antigen presentation cell- and T_{H}17 cell-mediated ocular immunoinflammatory disorder. In certain embodiments, the T_{H}17 cell-mediated ocular immunoinflammatory disorder is dry eye disease (DED).

The compositions embodied herein, comprise one or more NK1R antagonists.

In these and other embodiments, an NK1R antagonist comprises a small molecule antagonist of NK1R, a neutralizing anti-NK1R antibody, a blocking fusion protein against substance P (SP), an anti-SP antibody, a nucleic acid, or a polypeptide (e.g., an antibody or a soluble peptide such as the extracellular domain of a receptor or a ligand-binding portion thereof).

In certain embodiments, the NK1R antagonist is a small molecule. A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons. Small molecules may be, e.g., organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecule inhibitors can be synthetically engineered to consist of a fragment, or small portion, or a longer amino acid chain to fill a binding pocket of an enzyme. Typically small molecules are less than one kilodalton.

In certain embodiments, a pharmaceutical composition comprises an NK1R antagonist. For example, a NK1R peptide antagonist comprises Spantide (RPKPQQWFWLL; SEQ ID NO: 2). In some examples, the NK1R antagonist is a chemical compound, e.g., a small molecule antagonist. Exemplary NK1R small molecule antagonists are shown below.

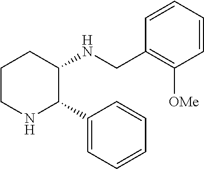 [SMILES: COc1ccccc1CN[C@H]1CCCN[C@H]1c1ccccc1]

(2S,3S)—N-[(2-Methoxyphenyl)methyl]-2-phenyl-3-piperidinamine dihydrochloride,

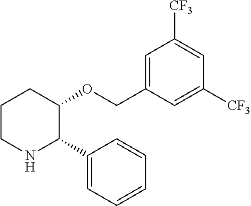 [SMILES: FC(F)(F)c1cc(CO[C@H]2CCCN[C@H]2c2ccccc2)cc(C(F)(F)F)c1]

(2S,3S)-3-[[3,5-bis(Trifluoromethyl)phenyl]methoxy]-2-phenylpiperidine hydrochloride,

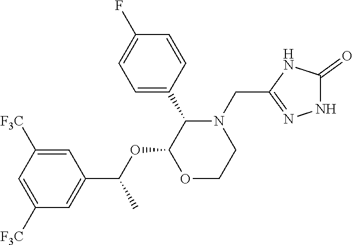 [SMILES: C[C@@H](O[C@H]1OCCN(Cc2n[nH]c(=O)[nH]2)[C@H]1c1ccc(F)cc1)c1cc(C(F)(F)F)cc(C(F)(F)F)c1]

5-[[(2R,3S)-2-[(1R)-[3,5-Bis(trifluoromethyl)phenyl]ethoxy]-3-(4-fluorophenyl)-4-morpholinylinyl]methyl]-1,2-dihydro-3H-1,2,4-triazol-3-one,

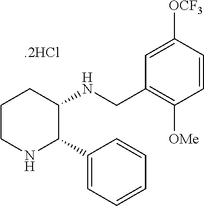 [SMILES: COc1ccc(OC(F)(F)F)cc1CN[C@H]1CCCN[C@H]1c1ccccc1]

(2S,3S)—N-[[2-Methoxy-5-trifluoromethoxy)pheny]methyl]-2-phenyl-3-piperidinamine dihydrochloride,

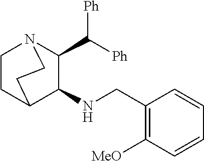 [SMILES: COc1ccccc1CN[C@H]1C2CCN(CC2)[C@H]1C(c1ccccc1)c1ccccc1]

(2S,3S)—N-(2-Methoxyphenyl)methyl-2-diphenylmethyl-1-azabicyclo[2.2.2]octan-3-amine,

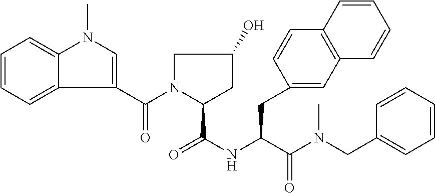 [SMILES: CN(Cc1ccccc1)C(=O)[C@H](Cc1ccc2ccccc2c1)NC(=O)[C@@H]1C[C@@H](O)CN1C(=O)c1cn(C)c2ccccc12]

(4R)-4-Hydroxy-1-[(1-methy-1H-indol-3-yl)carbonyl]-L-prolyl-N-methyl-3-2-naphthalenyl)-N-(phenylmethyl)-L-alaninamide,

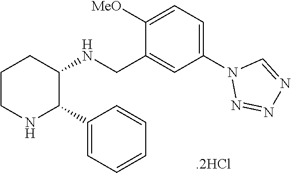 [SMILES: COc1ccc(-n2cnnn2)cc1CN[C@H]1CCCN[C@H]1c1ccccc1]

(2S,3S)—N-[[2-Methoxy-5-(1H-tetrazol-1-yl)phenyl]methyl]-2-phenyl-3-piperidinamine hydrochloride,

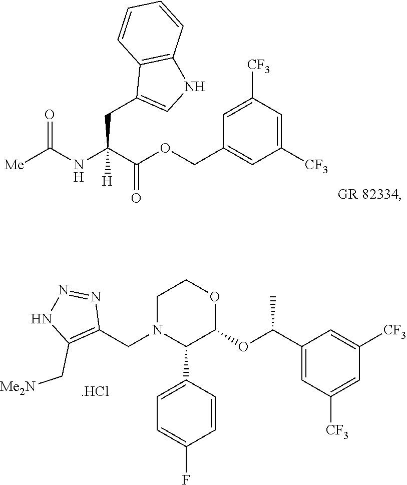

5-[[(2R,3S)-2[(1R)-1-[3,5-Bis(trifluoromethyl)phenyl]ethoxy]-3-(4-fluorophenyl)-4-morpholinyl]methyl-N,N-dimethyl-1H-1,2,3-triazole-4-methanamine hydrochloride,

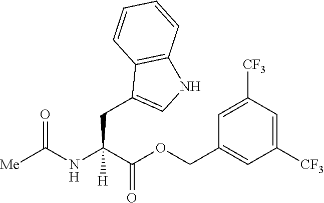

N-Acetyl-L-tryptophan 3,5-bis(trifluoromethyl)benzyl ester,

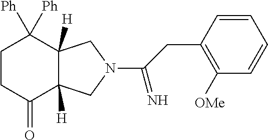 [SMILES: COc1ccccc1CC(=N)N1C[C@@H]2C(=O)CCC(c3ccccc3)(c3ccccc3)[C@@H]2C1]

(3aR,7aR)-Octahydro-2-[1-imino2-(2-methoxyphenyl)ethyl]-7,7-diphenyl-4H-isoindol,

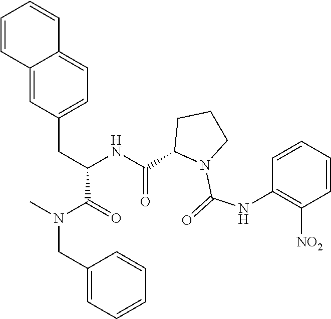 [SMILES: CN(Cc1ccccc1)C(=O)[C@H](Cc1ccc2ccccc2c1)NC(=O)[C@@H]1CCCN1C(=O)Nc1ccccc1[N+](=O)[O-]]

1-[[(2-Nitrophenyl)amino]carbonyl]-L-prolyl-N-methyl-3-2-naphthalenyl)-N-(phenylmethyl)-L-alaninamide,

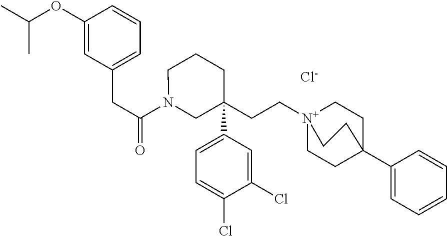 [SMILES: CC(C)Oc1cccc(CC(=O)N2CCC[C@](CC[N+]34CCC(c5ccccc5)(CC3)CC4)(c3ccc(Cl)c(Cl)c3)C2)c1 ; [Cl-]]

1-[2-[(3S)-3-(3,4-Dichlorophenyl)-1-[2-[3-(1-methylthoxy)phenyl]acetyl]-3-piperidinyl]ethyl]-4-phenyl-1-azoniabicyclo[2.2.2]octane chloride, or combinations thereof.

In certain embodiments, a pharmaceutical composition comprises an NK1R antagonist. In certain embodiments, the NK1R antagonist is selected from achiral pyridine class of neurokinin-1 receptor antagonists; netupitant 21; betctupitant 29; elzlopitant; lanepitant; osanetant; talnetant; GR205171; MK 0517; MK517; MEN 11467; nepadutant; MEN 11420; M274773; [Sar (9), Met (02) (11)]-Substance P; Tyr (6), D-Phe (7), D-His (9)-Substance-P (6-11) (sendide); (beta;-Ala(8))-Neurokinin A (4-10); (Tyr(5), D-Trp (6,8,9), Lys-NH(2) (10))-Neurokinin A; [D-Proz, D-Trip 7,9]-SP DPDT-SP; [D-Proz, D-Phe7, D-Trp9]-SP; SR48968 and 4-Alkylpiperidine derivative; telnetant; SB223412; SB223412A; telnetant hydrochloride; MDL103392; phosphorylated morpholine acetal human neurokinin-1 receptor agonists; SDZ NKT 343; LY 303 870; Ym-35375 and spiro-substituted piperidines; YM-44778; YM-38336; Septide; L732,13; Dactinomyan analogues; MEN 10207; L 659874; L 668,169; FR113680 and derivative; GR 83074; tripeptides possersi, the glutaminyl-D-tryptophyphenylalonite sequence; L 659,877; R396; Imidazo[4,5-b]quinoxaline cyonines as neurokinin antagonists; MEN 10208; DPDTP-octa; GR73632; GR64349; senktide; GR71251; [D-Argl, D-Pro2, D-Trp 7,9, Leul1]-SP (1-11); Ac heu-Asp-Gln-Trp-Phe-Gly NH2; Thr-Asp-Tyr-D-Tvp-Val-D-Trp-D-Trp-Arg NH2; Cyclo [Eln-Trp-Phe-Gly-Leu-Met]; D-Pro2D-Trp 7,9; D-Arg1D-Trp 7,9 leul1; [Gly6]-NKB [3-10]; [Arg3, D-Ala6]-NKB [3-10]; CP-9634; 3 aminoquinudidine; CP-99994; S18525; S19752; 4-quinoline carboxinide fremincik class; CP-122721; MK-869; GR205171; Spantide II; CP-96,345; L703,606; SR140, DNK333; 2-phenyl-4-quinolinecarboximides class; FK224; FR 115224; FK888; ZM253270-pyrrolopyrimidine class of nonpeptide neurokinin antagonists; GR71251; GR82334; RP67580; diacylpiperazine antagonists of human neurokinin e.g. L-161664; RP67580; MEN10376; GR98400; N2-[N2-(1H-indol-3-ylcarbonyl)-L-lysyl]-N-methyl-N-(phenyl-methyl)-L-phenylalaninamibe (2b); SP-(1-11); SP-(6-11); SP-(4-11) WIN51703; Spantide II; Spantide III; Spantide I; aprepitant; L754030; MK0869; ONO-7436; ONO 7436; MEN13510; 1-[2-(R)-{1-1R)-[3,5-bis(trifluoromethyl)phenyl]ethoxy}-3-(R)-(3,4-difluorophenyl)-4-(R)-tetrahydro-2H-pyran-4-ylmethyl]-3-(r)-methylpiperdine-3-carboxylic acid (1); LY 306,740; SLV-323; 2-substituted-4-aryl-6,7,8,9-tetrahydro-5H-pyrimido[4,5-b][1,5]oxazocin-5-one; 9-substituted-7-aryl-3,4,5,6-tetrahydro-2H-pyrido[4,3-b]- and [2,3-b]-1,5-oxazocin-6-one; SR142801; SB222200; CP96345; SR48968; ezlopitant; CJ 11974; MEN11558; [18F] SPA-RQ; neuropitant 21; betupitant 29; SR 144190; SR48692; SR141716; L733060; vofopitant; R-673; nepadutant; saredutant; UK 290795; 2-(4-biphenylyl)quinoline-4-carboxylate and carboxamide analogs (neurokinin-3 receptor antagonist); 4-Amino-2-(aryl)-butylbenzamides and analogues; MK-869; L742694; CP 122721; 1-alkyl-5-(3,4-dichlorophenyl)-5-[2-[(3-substituted)-1-azetidinyl- ]ethyl]-2-piperidines; L760735; L758,298, Cbz-Gly-Leu-Trp-0Bzl(CF(3))(2); L733,061; SR144190; SB235375; N-[(R,R)-(E)-1-arylmethyl-3-(2-oxo-azepan-3-yl)carbamoyl]allyl-N-methyl-3,5-bis(trifluoromethyl)benzamides; 3-[N¹-3,5-bis (trifluoromethyl)benzoyl-N-arylmethyl-N¹-methylhydrazino]-N-[(R)-2-oxo-azepan-3-yl]propionanides; SR142806; SR48,968; CP141,938; LY306740; SB40023; SB414240; Nolpitantium; SR140333; perhydroisoindole RP 67580, Depitant; RPR 100893; Lanepitant; LY-303870; sanoti synthelabo; nolpitanium; SR 140333; SR 48968; Savedutant; AV 608; AV-608, AV608; CGP 60829; NK-608; NKP-608C; NKP608; CS003; R113281; Vestipitant; 597599; GW 597599; GW 597599B; SSR 240600; casopitant; 679769; GW 679769; TA 5538; SSR 146977; SLV317; SLV-317; 823296; GW 823296; AVE 5883; AVE-5883; AZ 311; SB 235375; SB 733210; AZ 685; SAR 102279; SAR 10279; SSR 241586; SLV 332; Neurokinin 2 antagonist-Solvay; SLV-332; SLV332, NIK 616; MPV4505; NIK616; MPC 4505; Z501; Z-501; 1 TAK 637; CP 96345; L 659877; CGP 49823; GR 203040; L 732138; S 16474; WIN 51708; ZD 7944; S 18523; CI 1021; PD 154075; 758298; ZD 4974; S 18920; HMR 2091; FK 355; SCH 205528; NK 5807; NIP 531; SCH 62373; UK 224671; MEN 10627; WIN 64821; MDL 105212A; MEN 10573; TAC 363; 1 MEN 11149; HSP 117; NIP 530; AZD 5106 or combinations thereof.

In certain embodiments, the NK1R antagonist comprises CP-99,994, L-733,060 or spantide.

In certain embodiments, the NKiR antagonist is a nucleic acid molecule. Non-limiting examples of nucleic acid molecules include RNA interference-inducing (RNAi) molecules (e.g., siRNA, shRNA, miRNA, snoRNA), antisense oligonucleotides, aptamers (e.g., DNA aptamers and RNA aptamers). In certain embodiments, the nucleic acid is one selected from an aptamer, a small interfering RNA, a microRNA, a small hairpin RNA and an antisense nucleic acid.

Compositions of the present subject matter can be formulated in a variety of forms. In various embodiments, the composition is in the form of a solid, an ointment, a gel, a liquid, an aerosol, a mist, a polymer, a contact lens, a film, an emulsion, or a suspension. In some embodiments, the composition is administered topically. In preferred embodiments, treatment does not comprise systemic administration or substantial dissemination to non-ocular tissue.

In these and other embodiments, the composition is administered to the subject by a topical administration, a subconjunctival administration or an intravitreal administration. In certain embodiments, the compositions comprising one or more NK1R antagonists are administered subcutaneously for alleviating pain or pain management associated with ocular diseases, e.g. keratoneuralgia. The compositions can be injected subcutaneously in regions around the eye, e.g. forehead, eyelids etc. For example, the NK1R antagonist is subcutaneously injected 0.1, 0.2, 0.5, 1.0, 2.0, 3.0, 4.0, 5.0, 6.0, 7.0, 8.0, 9.0, 10.0 cm from the perimeter of the eye. The dosage and schedule for a subcutaneous administration may be the similar as described for topical administration or less frequently (as indicated for pain management). For subcutaneous administration to the skin of the eyelid or forehead of management of pain, e.g., for those who are diagnosed or suffering from Varicella zoster/shingles and suffering from chronic pain and redness, NK1R antagonist is administered one to 3 times per week for a period of approximately one to two months or for the duration of the pain associated with this disease.

In certain embodiments, the composition is topically administered to the subject at least once a day. In certain embodiments, the composition is topically administered to the subject at least twice a day. In certain embodiments, the composition is topically administered to the subject at least three times a day. In certain embodiments, the composition is ocularly administered to the subject. In certain embodiments, the composition is administered to the subject in combination with a secondary therapy or a secondary agent.

In certain embodiments, a pharmaceutical composition comprises an SP blocker, an SP antagonist, an SP receptor blocker, an SP receptor antagonist or combinations thereof as an active component, and a pharmaceutically acceptable carrier.

In various embodiments, the inhibited or antagonized expression or activity of SP and/or NK1R is reduced by about 10%, 15%, 20%, 25%, 30%, 35%, 40%, 45%, 50%, 55%, 60%, 65%, 70%, 75%, 80%, 85%, 90%, 95%, 99% or more compared to the level of expression or activity in a control. In some embodiments, the inhibited expression or activity is between about 10% and about 25%, about 10% and about 50%, about 10% and about 75%, about 1% and about 50%, about 1% and about 25%, about 25% and about 50%, 50% and about 75% or any other range between 0.5% and 99% the level of expression or activity in a control.

In these and other embodiments, a symptom of the ocular immunoinflammatory disorder is reduced within about 5, 15, 30, or 60 minutes; or within 1, 2, 3, 4, 5, 6, 7, 8, 9, 10, 11, 12, 13, or 14 days after administration of an inhibitor. In some embodiments, a composition is administered to the eye of the subject, less than 1, 2, 3, 4, 5, or 6 times per day, about 1, 2, 3, 4, 5, 6, or 7 times per week; or once daily. In certain embodiments, the composition is administered by the subject (i.e., self-administration) or by a physician. Aspects of the present subject matter provide methods for treating a subject afflicted with an ocular immunoinflammatory disorder. Such methods include locally administering to an eye of the subject a composition having an effective amount of an NK1R antagonist so as to thereby treat the subject.

Aspects of the present disclosure also provide contact lens comprising a composition comprising an effective amount of an NK1R and/or SP antagonist. The composition is incorporated into or coated onto the lens. Devices including a polymer and a bioactive composition having an effective amount of an NK1R and/or SP antagonist are also provided. In various embodiments, the device is for delivery into or onto an ocular tissue. For example, the device contacts an ocular tissue. In certain embodiments, such devices are implanted or injected into an ocular tissue or fluid cavity. Aspects of the present invention further provide compositions with an effective amount of an NK1R and/or SP antagonist or inhibitor in an ophthalmically acceptable vehicle.

In certain embodiments, an eye drop composition comprises a therapeutically effective amount of an NK1R antagonist and a pharmaceutically acceptable carrier. In certain embodiments, a composition is within a dispenser suitable for administering a drop of the composition to an eye of a subject. In certain embodiments, the composition has an osmolarity between about 200 to about 400 milliosmoles/kilogram inclusive and a pH from about 6.5 to about 7.5 inclusive.

In certain embodiments, the composition comprises a neutralizing or function-blocking antibody against NK1R and/or SP and/or against NK1R/SP interactions. The neutralizing or function-blocking antibody may be a reformulated or humanized derivative of or bind to the epitope of an affinity-purified polyclonal antibody.

Exemplary methods for inhibiting or reducing the severity of an ocular immunoinflammatory disease may be carried out by locally administering to an eye of a subject a composition comprising a polynucleotide, a polypeptide, an antibody, a compound, or a small molecule that inhibits or modifies the transcription, transcript stability, translation, modification, localization, secretion, interaction, binding or function of a polynucleotide or polypeptide encoding NK1R or SP and/or any component of NK1R or SP.

In various embodiments, a composition comprises a ribozyme, an antisense oligonucleotide (such as a morpholino), a microRNA (miRNA), a short hairpin RNA (shRNA), or a short interfering RNA (siRNA) to reduce or silence gene expression.

In certain embodiments, the composition may comprise an intrabody that binds to NK1R or SP. The composition may alternatively, or in addition, comprise a soluble fragment of SP or mimetic thereof which binds NK1R but does not induce signaling. Exemplary polypeptides include, but are not limited to, fusion and/or chimeric proteins capable of disrupting SP/NK1R function.

In some embodiments, function-blocking antibodies targeted against NK1R or SP are monoclonal or polyclonal. Antibodies include those that bind to one or more sequences within NK1R receptor polypeptide. In certain embodiments, the antibody is an intrabody. In some embodiments, the antibody comprises a single chain, a humanized, a recombinant, or a chimeric antibody.

In certain embodiments, the NK1R antagonist is administered in combination with a second therapeutic agent or treatment. The NK1R antagonist is administered either simultaneously or sequentially with a secondary composition comprising one or more of the following: an antibiotic, an immunosuppressive composition, an anti-inflammatory composition, a growth factor, a steroid, a chemokine, or a chemokine receptor.

Other embodiments are described infra.

Each embodiment disclosed herein is contemplated as being applicable to each of the other disclosed embodiments. Thus, all combinations of the various elements described herein are within the scope of the invention.

### General Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, the term “about” in the context of a numerical value or range means ±10% of the numerical value or range recited or claimed, unless the context requires a more limited range. Unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments,±100% in some embodiments±50%, in some embodiments±20%, in some embodiments±10%, in some embodiments±5%, in some embodiments ±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

As used herein, the terms “aptamer(s)” or “aptamer sequence(s)” are meant to refer to single stranded nucleic acids (RNA or DNA) whose distinct nucleotide sequence determines the folding of the molecule into a unique three dimensional structure. Aptamers comprising 15 to 120 nucleotides can be selected in vitro from a randomized pool of oligonucleotides (1014-1015 molecules). Aptamers that bind to pre-selected targets including proteins and peptides with high affinity and specificity can be designed and/or selected using methods known in the art. See, e.g., Cox, J. C.; Ellington, A. D. (2001) Bioorganic & Medicinal Chemistry 9 (10): 2525-2531; Cox, J. C.; Hayhurst, A.; Hesselberth, J.; Bayer, T. S.; Georgiou, G.; Ellington, A. D. (2002) Nucleic Acids Research 30 (20): e108.; and Neves, M.A.D.; O. Reinstein; M.Saad; P.E. Johnson (2010) Biophys Chem 153 (1): 9-16, the entire content of each of which is incorporated herein by reference.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In various embodiments, a comparison window is the entire length of one or both of two aligned sequences. In some embodiments, two sequences being compared comprise different lengths, and the comparison window is the entire length of the longer or the shorter of the two sequences. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

In various embodiments, an algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information, as known in the art. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

For sequence comparison, typically one sequence acts as a reference sequence, to which test sequences are compared. In various embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

The term “enhancement,” “enhance,” “enhances,” or “enhancing” refers to an increase in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or an increase in the specified activity of at least about 5%, 10%, 25%, 35%, 40%, 50%, 60%, 75%, 80%, 90%, 95%, 97%, 98%, 99% or 100%.

As used herein, the term “immune cells” generally includes white blood cells (leukocytes) which are derived from hematopoietic stem cells (HSC) produced in the bone marrow “Immune cells” includes, e.g., lymphocytes (T cells, B cells, natural killer (NK) cells) and myeloid-derived cells (neutrophil, eosinophil, basophil, monocyte, macrophage, dendritic cells).

The term “immune effector cell,” as used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NK-T) cells, mast cells, and myeloic-derived phagocytes. “Immune effector function or immune effector response,” as that term is used herein, refers to function or response, e.g., of an immune effector cell, that enhances or promotes an immune attack of a target cell. For example, an immune effector function or response refers a property of a T or NK cell that promotes killing or the inhibition of growth or proliferation, of a target cell. In the case of a T cell, primary stimulation and co-stimulation are examples of immune effector function or response.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. Such sequences that are at least about 80% identical are said to be “substantially identical.” In some embodiments, two sequences are 100% identical. In certain embodiments, two sequences are 100% identical over the entire length of one of the sequences (e.g., the shorter of the two sequences where the sequences have different lengths). In various embodiments, identity may refer to the complement of a test sequence. In some embodiments, the identity exists over a region that is at least about 10 to about 100, about 20 to about 75, about 30 to about 50 amino acids or nucleotides in length. In certain embodiments, the identity exists over a region that is at least about 50 amino acids in length, or more preferably over a region that is 100 to 500, 100 to 200, 150 to 200, 175 to 200, 175 to 225, 175 to 250, 200 to 225, 200 to 250 or more amino acids in length.

The term “inhibit,” “diminish,” “reduce” or “suppress” refers to a decrease in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or a decrease or reduction in the specified activity of at least about 5%, 10%, 25%, 35%, 40%, 50%, 60%, 75%, 80%, 90%, 95%, 97%, 98%, 99% or 100%. These terms are intended to be relative to a reference or control.

As used herein, an “isolated” or “purified” nucleic acid molecule, polynucleotide, polypeptide, or protein, or chemical compound is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) or polypeptide is free of the genes or sequences that flank it in its naturally-occurring state. A purified or isolated peptide or protein fragment does not contain amino acids that flank the sequence of the peptide or protein fragment in the naturally-occurring full-length reference protein. The reference sequence is identified by a SEQ ID number. Purified also defines a degree of sterility that is safe for administration to a human subject, e.g., lacking infectious or toxic agents.

The term “metabolite” includes any compound into which the active agents can be converted in vivo once administered to the subject. Examples of such metabolites are glucuronides, sulphates and hydroxylates.

As used herein, “modulate,” “modulates” or “modulation” refers to enhancement (e.g., an increase) or inhibition (e.g., diminished, reduced or suppressed) of a specified activity. Modulation can increase activity more than 1-fold, 2-fold, 3-fold, 5-fold, 10-fold, 100-fold, etc., over baseline values. Modulation can also decrease its activity below baseline values. Modulation can also normalize an activity to a baseline value.

As used herein, “NK antagonist” is intended to encompass known and as yet unknown compounds (including pharmaceutically acceptable salts, derivatives, homologs or analogs thereof) which inhibit, decrease or block, or otherwise impair the activity of neurokinin 1, neurokinin 2 or substance P. Such compounds can act directly on neurokinin 1, neurokinin 2 or substance P to inhibit its activity or can act on the family of NK receptors such as NK1, NK2 or NK3 receptors. Examples of such agents include achiral pyridine class of neurokinin-1 receptor antagonists; aprepitant; netupitant 21; betctupitant 29; elzlopitant; lanepitant; osanetant; talnetant; GR205171; MK 0517; MK517; MEN 11467; nepadutant; MEN 11420; M274773; [Sar (9), Met (02) (11)]-Substance P; Tyr (6), D-Phe (7), D-His (9)-Substance-P (6-11) (sendide); (β-Ala(8))-Neurokinin A (4-10); (Tyr(5), D-Trp (6,8,9), Lys-NH(2) (10))-Neurokinin A; [D-Proz, D-Trip 7,9]-SP DPDT-SP; [D-Proz, D-Phe7, D-Trp9]-SP; SR48968 and 4-Alkylpiperidine derivative; telnetant; SB223412; SB223412A; telnetant hydrochloride; MDL103392; phosphorylated morpholine acetal human neurokinin-1 receptor agonists; SDZ NKT 343; LY 303 870; Ym-35375 and spiro-substituted piperidines; YM-44778; YM-38336; Septide; L732,13; Dactinomyan analogues; MEN 10207; L 659874; L 668,169; FR113680 and derivative; GR 83074; tripeptides possersi, the glutaminyl-D-trypto phy phenyl alonite sequence; L 659,877; R396; Imidazo[4,5-b]quinoxaline cyonines as neurokinin antagonists; MEN 10208; DPDTP-octa; GR73632; GR64349; senktide; GR71251; [D-Argl, D-Pro2, D-Trp 7,9, Leull]-SP (1-11); Ac heu-Asp-Gln-Trp-Phe-Gly NH2; Thr-Asp-Tyr-D-Tvp-Val-D-Trp-D-Trp-Arg NH2; Cyclo [Eln-Trp-Phe-Gly-Leu-Met]; D-Pro2D-Trp 7,9; D-Arg1D-Trp 7,9 leull; [Gly6]-NKB [3-10]; [Arg3, D-Ala6]-NKB [3-10]; CP-9634; 3 aminoquinudidine; CP-99994; 518525; 519752; 4-quinoline carboxinide fremincik class; CP-122721; MK-869; GR205171; Spantide II; CP-96,345; L703,606; SR140, DNK333; 2-phenyl-4-quinolinecarboximides class; FK224; FR 115224; FK888; ZM253270-yrrolopyrimidine class of nonpeptide neurokinin antagonists; GR71251; GR82334; RP67580; diacylpiperazine antagonists of human neurokinin eg L-161664; RP67580; MEN10376; GR98400; N2-[N2-(1H-indol-3-ylcarbonyl)-L-lysyl]-N-methyl-N-(phenyl-methyl)-L-phen- ylalaninamibe (2b); SP-(1-11); SP-(6-11); SP-(4-11) WIN51703; Spantide II; Spantide III; Spantide I; L754030; MK0869; ONO-7436; ONO 7436; MEN13510; 1-[2-(R)-{1-1R)-[3,5-bis(trifluoromethyl)phenyl]ethoxy}-3-(R)-(3,4-difluorophenyl)-4-(R)-tetrahydro-2H-pyran-4-ylmethyl]-3-(r)-methylpiperidine-3-c- arboxylic acid (1); LY 306,740; SLV-323; 2-substituted-4-aryl-6,7,8,9-tetrahydro-5H-pyrimido[4,5-b][1,5]oxazocin-5-one; 9-substituted-7-aryl-3,4,5,6-tetrahydro-2H-pyrido[4,3-b]- and [2,3-b]-1,5-oxazocin-6-one; SR142801; SB222200; CP96345; SR48968; ezlopitant; CJ 11974; MEN11558; [18F] SPA-RQ; neuropitant 21; betupitant 29; SR 144190; SR48692; SR141716; L733060; vofopitant; R-673; nepadutant; saredutant; UK 290795; 2-(4-biphenylyl)quinoline-4-carboxylate and carboxamide analogs (neurokinin-3 receptor antagonist); 4-Amino-2-(aryl)-butylbenzamides and analogues; MK-869; L742694; CP 122721; 1-alkyl-5-(3,4-dichlorophenyl)-5-[2-[(3-substituted)-1-azetidinyl-]ethyl]-2-piperidines; L760735; L758,298, Cbz-Gly-Leu-Trp-0Bzl(CF(3))(2); L733,061; SR144190; SB235375; N-[(R,R)-(E)-1-arylmethyl-3-(2-oxo-azepan-3-yl) carbamoyl]allyl-N-methyl-3,5-bis(trifluoromethyl)benzamides; 3-[N¹-3,5-bis(trifluoromethyl)benzoyl-N-arylmethyl-N¹-methylhydrazino-9 -N-[(R)-2-oxo-azepan-3-yl]propionanides; SR142806; SR48,968; CP141,938; LY306740; SB40023; SB414240; Nolpitantium; SR140333; perhydroisoindole RP 67580, Depitant; RPR 100893; Lanepitant; LY-303870; LY303870; sanoti synthelabo; nolpitanium; SR 140333; SR 48968; Savedutant; AV 608; AV-608, AV608; CGP 60829; NK-608; NKP-608C; NKP608; CS003; R113281; Vestipitant; 597599; GW 597599; GW 597599B; Neurokinin antagonist; SSR 240600; casopitant; 679769; GW 679769; TA 5538; SSR 146977; SLV317; SLV-317; 823296; GW 823296; AVE 5883; AVE-5883; AZ 311; SB 235375; SB 733210; AZ 685; SAR 102279; SAR 10279; SSR 241586; SLV 332; Neurokinin 2 antagonist-Solvay; NK-2 antagonist-Solvat; SLV-332; SLV332, NIK 616; MPV4505; NIK616; MPC 4505; Z501; Z-501; 1 TAK 637; CP 96345; L 659877; CGP 49823; GR 203040; L 732138; S 16474; WIN 51708; ZD 7944; S 18523; CI 1021; PD 154075; 758298; ZD 4974; S 18920; HMR 2091; FK 355; SCH 205528; NK 5807; NIP 531; SCH 62373; UK 224671; MEN 10627; WIN 64821; MDL 105212A; MEN 10573; TAC 363; 1 MEN 11149; HSP 117; NIP 530; and AZD 5106.

As used herein, the term “NK-1 receptor” is used as commonly understood in the art to refer to the mammalian receptor also referred to as the tachykinin NK-1 receptor, which is a 407 amino acid protein having a molecular weight of 58.000, and is a member of family 1 (rhodopsin-like) of the G protein-coupled receptors, and conservative variants thereof.

As used herein, the term “NK-1 Receptor Antagonist” refers to a compound that selectively binds to the NK-1 receptor and reduces or eliminates the biological activity thereof. For example, selective binding refers to about a 2-fold to 10,000-fold higher affinity of the antagonist to the NK-1 receptor relative to its affinity to either the NK-2 receptor or the NK-3 receptor. For example, the NK1R antagonist binds to the NK-1 receptor with a 2-fold, 5-fold, 10-fold, 50-fold 100-fold, 1000-fold, 5000-fold, 10,000-fold or more affinity compares to NK-2 or NK-3 receptor.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject. Such a carrier or excipient does not comprise NK1R antagonist activity.

As used herein, the term “prodrug” refers to a compound that is a precursor of another compound that is a pharmacologically active agent, wherein the precursor compound is administered to a subject in an inactive form and once administered is metabolized in vivo into the pharmacologically active agent.

A small molecule is a compound that is less than 2000 Daltons in mass. The molecular mass of the small molecule is preferably less than 1000 Daltons, more preferably less than 600 Daltons, e.g., the compound is less than 500 Daltons, 400 Daltons, 300 Daltons, 200 Daltons, or 100 Daltons.

The terms “subject,” “patient,” “individual,” and the like as used herein are not intended to be limiting and can be generally interchanged. That is, an individual described as a “patient” does not necessarily have a given disease, but may be merely seeking medical advice. The term “subject” as used herein includes any member of the animal kingdom, such as a mammal. In one embodiment, the subject is a human. In another embodiment, the subject is a mouse.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

As used herein, a “symptom” associated with a disorder includes any clinical or laboratory manifestation associated with the disorder, and is not limited to what the subject can feel or observe.

As used herein, “treating” encompasses, e.g., inhibition, regression, stasis of the progression, or reduction of the severity of a disorder or disease. Treating also encompasses the prevention or amelioration of any symptom or symptoms of the disorder. As used herein, “inhibition” of disease progression or a disease complication in a subject means preventing or reducing the disease progression and/or disease complication, signs, or symptoms in the subject.

GenBank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited. For example, where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

Each embodiment disclosed herein is contemplated as being applicable to each of the other disclosed embodiments. Thus, all combinations of the various elements described herein are within the scope of the invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### DESCRIPTION OF THE DRAWINGS

FIGS. 1A-1C are graphs showing the increased level of substance P (SP) in the DED mouse model.

FIG. 2 is a diagram showing mechanisms of blocking SP-NK1R signaling in treating ocular immunoinflammatory disorders.

FIGS. 3A and 3B are graphs showing that NK1R antagonist abrogates SP-mediated Regulatory T cell (Treg) decrease.

FIG. 4 is a graph showing that Substance P levels increase in the ocular surface in the course of DED. DED was induced in C57BL/6 mice for 14 days. Trigeminal ganglia (TGs) of control (day 0) and DED mice (day 4) were harvested and tissues were homogenized.. Protein levels of substance P in the TG were analyzed in the supernatant of homogenized tissues of control and DED mice using ELISA, and protein levels were adjusted to the total protein measured by the BCA Protein assay kit. Data represent mean±SEM of two independent experiments. *p<0.05.

FIGS. 5A and 5B are graphs showing that Substance P derived from trigeminal ganglion promotes bone marrow-derived dendritic cell maturation. FIG. 5A. Bone marrow derived dendritic cells (BMDCs) were cultured in the presence of different doses of substance P (25, 50 and 100 μg/mL) for 18 hours. Maturation of BMDCs was evaluated by assessing the expression (mean fluorescence intensity [MFI]) of MHC II using flow cytometry. FIG. 5B. Trigeminal ganglions (TGNs) harvested from either DED or control mice were cultured for 3 days, and were subsequently co-cultured with BMDCs for 24 hours. To block the effect of TGN-derived SP on BMDCs, neurokinin-1-receptor antagonist Spantide (10 μM and 100 μM) was added to the co-culture system. Expression of MHC II by CD11e BMDCs was assessed in different groups using flow cytometry. Data represent mean±SEM of two independent experiments. MFI: mean fluorescence intensity. *p<0.05.

FIGS. 6A-6E are a series of graphs and a scatter plot showing that topical blockade of NK1R inhibits antigen-presenting cell maturation and ameliorates dry eye disease. After induction of DED for 4 days, mice were topically treated with NK1R antagonists, CP-99,994 or L-733,060 (1 μg/p1), or PBS (as the control) three times a day until day 14 after DED induction. Untreated DED mice served as controls. (FIG. 6A) Corneal fluorescein staining (CFS) was performed on days 4, 7, 10 and 14 to assess disease severity. Frequencies of mature MHC II^{hi} CD11b⁺ antigen presenting cells (APCs) in the cornea (FIG. 6B) and draining lymph nodes (FIGS. 6C & 6D), and expression of MHC II by APCs in the draining lymph nodes (FIG. 6E) on day 14 were assessed in different groups using flow cytometry. MFI: mean fluorescence intensity. Data represent mean±SEM of two independent experiments. *p<0.05.

FIGS. 7A-7D are a series of graphs and plots showing that topical blockade of NK1R suppresses activation of T_{H}17 cells in draining lymph nodes and their infiltration in the conjunctivae. After induction of DED for 4 days, mice received topical treatment with NK1R antagonists, CP-99,994 or L-733,060 (1 μg/p1), or PBS (as the control) three times a day until day 14 after DED induction. Untreated DED mice served as the control. (FIG. 7A) Frequencies of CD4⁺ IL-17⁺ T_{H}17 cells in the DLN of naïve, DED mice treated with CP-99,994, L-733,060, or PBS and untreated DED mice were evaluated on day 14 after DED induction using flow cytometry. (FIGS. 7B & 7C) Frequencies of T_{H}17 cells in the conjunctivae of naïve, NK1R antagonist-treated and untreated DED mice, and (FIG. 7D) mRNA expression levels of IL-17 in the conjunctivae of different groups were assessed on day 14 using flow cytometry and real-time PCR, respectively. Data represent mean±SEM of two independent experiments. Conj: conjunctivae. *p<0.05.

FIGS. 8A-8C are a series of graphs showing that substance P induces Treg dysfunction by suppressing Treg expression of inhibitory molecules and secreted immunoregulatory cytokines, which is reversed by Spantide I. FIG. 8A is a series of graphs demonstrating that SP suppresses the expression of inhibitory molecules Foxp3, CTLA4 and PD-1 by Tregs in vitro. In addition, incubation of Tregs with SP results in (FIG. 8B) secretion of significantly lower levels of immunoregulatory cytokines (TGFβ and IL-10) by Tregs and (FIG. 8C) a lower capacity of Tregs to suppress effector T cell proliferation in vitro. However, addition of NK-1R antagonist, Spantide I (Spt), to the co-culture reverses SP-induced Treg dysfunction. Data represent mean±SEM. *p<0.05.

FIGS. 9A-9D are a series of graphs and plots demonstrating that inhibition of SP signaling through systemic administration of Spantide I restores Treg function, suppresses T_{H}17 cells and reduces the severity of DED. FIG. 9A: DED mice treated with Spantide I (Spt) had significantly lower corneal fluorescein staining (CFS) scores compared to PBS-treated controls. FIG. 9B: Tregs isolated from DLN of DED mice treated with Spantide I had a higher capacity in suppressing effector T cell proliferation than Tregs derived from the control group. In addition, treatment of DED mice with Spantide I led to a significant reduction in frequencies of CD4⁺L-17⁺ T_{H}h17 cells in (FIG. 9C) draining lymph nodes and (FIG. 9D) conjunctivae of DED mice compared to the control. Data represent mean±SEM. *p<0.05 **p<0.001.

FIG. 10 is a series of photographic images demonstrating an evaluation of ocular redness in animal models using Ocular Redness Index (ORI) with Image J. Step 1: The conjunctival image was captured by means of a digital camera and was recorded as an RGB color JPEG image (3264×2448 pixels). Step 2: To reduce the background noise in the images, a color correction (white balance) was made according to selected filter paper area (black box). Step 3: An ROI (Region Of Interest, area inside the yellow circle) was selected as the evaluation area by user, and the program read the red-green-blue (RGB) values of each pixel, converting it to huesaturation-value (HSV) space, and converted these values into a numeric centesimal value for redness in the selected area automatically (Image J computer program; NIH).

FIG. 11 is a series of images, a table and a graph demonstrating results obtained from a rabbit model of non-allergic (non-infectious) ocular redness. Animals were anesthetized, and 40 ᵢll of a series of increasing concentrations of dapiprazole was applied to the right eyes of animals to induce ocular redness. PBS was used as a control on the left eyes. Eyes were examined by a slit lamp, and images were taken pre-induction, and every 30 seconds for the first 2 minutes post-induction, followed by every 4 minutes for additional 8 minutes and every 10 minutes until 1 hour. The table summarizes the maximum change in ORI scores post-induction and the time when such change happened (peak time) during the 1-hour observing period. Representative images show the development of ocular redness, and the graph summarizes the kinetics of change in ORI score with data representing mean±SEM. Results show the 5% dapiprazole inducing the highest increase in ORI score. *, p<0.05, †, p<0.01 for 5% dapiprazole vs. PBS.

FIG. 12 is a series of images, a table and a graph demonstrating results obtained from a Guinea pig model of allergic ocular redness. Animals were anesthetized, and 20 μl of 1.5 mg/ml histamine was applied to the right eyes of animals to induce ocular redness. PBS was used as a control on the left eyes. Eyes were examined by a slit lamp, and images were taken pre-induction, and every 30 seconds for the first 2 minutes post-induction, followed by every 4 minutes for additional 8 minutes and every 10 minutes until 1 hour. The table summarizes the maximum change in ORI scores post-induction and the time when such change happened (peak time) during the 1-hour observing period. Representative images show the development of ocular redness, and the graph summarizes the kinetics of change in ORI score with data representing mean±SEM. †, p<0.01 as compared to PBS.

FIG. 13 is a graph and a series of images demonstrating that neurokinin-1 receptor antagonism ameliorates non-allergic ocular redness. Animals were topically treated with 1.0 mg/ml L-703,606 (a highly selective NK1R antagonist) at the same time of 5% dapiprazole instillation in 40 μl volume. Animals induced with dapiprazole but without L-703,606 treatment served as the control. Eyes were examined by a slit lamp, and images were taken pre-induction, and every 30 seconds for the first 2 minutes post-induction, followed by every 4 minutes for additional 8 minutes and every 10 minutes until 1 hour. The kinetics of change in ORI score with data representing mean±SEM is summarized. Results show blockade of SP/NK1R rapidly and consistently ameliorating the ocular redness. *, p<0.05, †, p<0.01 as compared to control.

FIG. 14 is a graph and a series of images demonstrating that neurokinin-1 receptor antagonism ameliorates allergic ocular redness. Animals were topically treated with 1.0 mg/ml L-703,606 (a highly selective NK1R antagonist) at the same time of 1.5 mg/ml histamine instillation in 20 μl volume. Animals induced with histamine but without L-703,606 treatment served as the control. Eyes were examined by a slit lamp, and images were taken pre-induction, and every 30 seconds for the first 2 minutes post-induction, followed by every 4 minutes for additional 8 minutes and every 10 minutes until 1 hour. The kinetics of change in ORI score with data representing mean±SEM is summarized. Results show blockade of SP/NK1R rapidly and consistently ameliorating the ocular redness. *, p<0.05, †, p<0.01 as compared to control.

FIGS. 15A and 15B are a series of graphs showing that substance P levels increase in the ocular surface in the course of dry eye disease (DED). DED was induced in C57BL/6 mice for 14 days. Corneas, conjunctiva, and trigeminal ganglia (TGs) of control (day 0) and DED mice (days 4 and 14) were harvested; and tissues were homogenized. FIG. 15A: Protein levels of substance P in the cornea, conjunctiva, and TG were analyzed in the supernatant of homogenized tissues of control and DED mice using enzyme-linked immunosorbent assay; and protein levels were adjusted to the total protein measured by the BCA Protein assay kit (Thermo Scientific, Rockford, Ill.). FIG. 15B: Substance P mRNA levels in the cornea, conjunctiva, and TG of control and DED mice were analyzed using real-time PCR. Data are expressed as means±SEM (FIGS. 15A and 15B). n=2 independent experiments (FIGS. 15A and 15B).

FIGS. 16A-16C are schematic representations of non-peptide small molecule compounds useful in the methods described herein. Each of these non-peptide compounds shares similar structures, consisting of three elements: (a) a piperidine or quinuclidine ring with a bridgehead nitrogen; (b) a benzhydryl group; and (c) a benzyl amino side chain. All three elements are needed for the interactivity with NK1R. L-733,060 is structurally closest to MK-869 (L-754,030)/aprepitant, the first FDA-approved NK1R antagonist indicated for chemotherapy-induced nausea and vomiting. The drug has also been used for migraine or depression. Poor water solubility of MK-869 prevents its use as an eye drop formulation. FIG. 16A is a schematic representation showing the molecular structure of CP-99,994 (Pfizer). FIG. 16B is a schematic representation showing the molecular structure of L-733,060 (Merck), an analogue of CP-99,994. FIG. 16C is a schematic representation showing the molecular structure of L-703,060 (Merck), an analogue of CP-96,345. L-703,060 has a higher NK1R affinity than CP-96,345. Instead of a piperidine ring, L-703,060 has a benzylamino quinuclidine structure.

FIG. 17 is a schematic representation showing the molecular structure of dapiprazole.

### DETAILED DESCRIPTION

The present disclosure relates to a method or the treatment of substance P (SP)-associated, non-infectious ocular disorders, including Dry Eye Disease (DED), ocular redness, allergic conjunctivitis, and ocular pain, comprising ocular delivery (e.g., topical, subconjunctival, or intravitreal administration) of a blocker or antagonist to SP or SP receptor (e.g., neurokinin 1 receptor, NK1R) of the antagonist in combination with a suitable vehicle preparation. The ‘SP or NK1R blocker or antagonist’ comprises any agent able to suppress SP receptor-mediated signal transduction, and may include, but is not restricted to the following: a small molecule antagonist of NK1R, a neutralizing anti-NK1R antibody, a blocking fusion protein or antibody directed against SP, or any other agents (such as a DNA aptamer, an RNA aptamer, or an oligonucleotide) that reduce the expression, or signaling mediated by, NK1R or SP. Suppresses SP-associated inflammatory responses in non-infectious ocular surface diseases including, but not limited to, DED, ocular redness, allergic conjunctivitis, and ocular pain.

The ocular surface (cornea and conjunctiva) is the most innervated tissue in the body. Among various nerve-derived factors, substance P (SP), an 11-amino acid neuropeptide, serves as an active mediator of inflammation (J Cell Physiol, 2004; 201:167-180). SP blockade has been shown to reduce the severity in corneal infections (Invest Ophthalmol Vis Sci. 2008; 49:4458-4467; Invest Ophthalmol Vis Sci. 2011; 52:8604-8613) and neovascularization (Invest Ophthalmol Vis Sci, 2014; 55:6783-6794). However, little is known about the role of SP in the pathogenesis of non-infectious ocular surface disorders, such as DED and ocular redness (non-DED-related).

SP-associated, non-infectious ocular disorders are highly prevalent. For example, DED, which is characterized by chronic ocular surface inflammation, is the most frequent non-refractive reason leading patients to seek professional eye care (Am J Ophthalmol. 2007; 143:409-15). DED is estimated to affect 10-20% of the adult population (Ocul Surf 2007; 5:75-92) and approximately 5 million Americans over the age of 50 years, with millions more experiencing intermittent symptoms of dry eye (Ocul Surf 2007; 5:93-107). The prevalence in women is nearly two times higher than that in men (Am J Ophthalmol. 2003; 136:318-26; Arch Ophthalmol. 2009; 127:763-8). The disease has an adverse effect on vision-related quality of life and productivity, and has caused a considerate economic burden on public health (Ocul Surf 2017; 15:334-65). The therapeutic strategies have been restricted to symptomatic relief with various types of lubricating drops and ointments, which do not address the underlying disease process, and non-specific anti-inflammatory treatments with corticosteroid, which is limited for the long-term usage due to the sight-threatening side effects of raised intraocular pressure and cataracts (Curr Opin Ophthalmol 2000; 11:478-483). For example, non-specific anti-inflammatory therapies are the mainstay of treatment for moderate to severe DED, along with topical cyclosporine and lifitegrast. Despite the recent advent of two FDA-approved therapeutics, topical cyclosporine (RESTASIS®) and lifitegrast (XIIDRA®), there remains an unmet need for immunomodulatory agents that focus on targeting specific components of the underlying immune response in DED.

Ocular redness is even more prevalent, and most people experience red eyes at some point. In a study, 9 out of 10 subjects reported self-medicating for ocular redness. So far only subjective quantification of ocular redness severity is used clinically (Efron Nathan, et al. “Validation of Grading Scales for Contact Lens Complications.” Ophthalmic and Physiological Optics, vol. 21, no. 1, 2001, pp. 17-29; Schulze, Marc M., et al. “The Development of Validated Bulbar Redness Grading Scales.” Optometry and Vision Science, vol. 84, no. 10, 2007, pp. 976-983; Schulze, Marc M., et al. “The Perceived Bulbar Redness of Clinical Grading Scales.” Optometry and Vision Science, vol. 86, no. 11, 2009, pp. E1250-E1258). Ocular redness is one of the most commonly seen signs in ophthalmological clinic, and is commonly due to the dilation of conjunctival blood vessels with infectious and non-infectious causes (Invest Ophthalmol Vis Sci. 2013; 54:4821-4826). Ocular redness is characterized by reactive dilatation of the conjunctival blood vessels, resulting in hyperemia of the conjunctiva (Leibowitz, Howard M. “The Red Eye.” The New England Journal of Medicine, vol. 343, no. 5, 2000, pp. 345-351; Amparo, et al. “The Ocular Redness Index: A Novel Automated Method for Measuring Ocular Injection.” Investigative Ophthalmology & Visual Science, 2013, pp. Quick submit: 2017-06-18T21:14:33-0400; McLaurin, Eugene, et al. “Brimonidine Ophthalmic Solution 0.025% for Reduction of Ocular Redness: A Randomized Clinical Trial.” Optometry and Vision Science, vol. 95, no. 3, 2018, pp. 264-271). Among the non-infectious ocular redness, DED and allergy are the two typical underlying disorders (Clin Ophthalmol. 2013; 7:1197-1204; Curr Eye Res. 2018; 43:43-5.). Treatment of ocular redness depends on the underlying cause. Antihistamines and mast cell stabilizers are currently used for mild allergic conjunctivitis, and topical steroids are required for the server form. The long-term usage of corticosteroids is limited due to its significant side effects. For non-allergic redness, topical vasoconstrictor agents are commonly used. For example, current treatments for non-infectious ocular redness mainly include the over-the-counter (OTC) eye drops containing vasoconstrictors, such as CLEAR EYES® and VISINE®. However, their efficacy is limited due to tachyphylaxis (tolerance or loss of effectiveness), redness rebound upon treatment discontinuation (worsening of condition as compared to baseline), and systemic side effects (Curr Eye Res. 2018; 43:43-45). More recently, a more selective vasoconstrictor—LUMIFY® (0.025% brimonidine) has been approved by FDA as an OTC to treat ocular redness. But, the medication itself can cause ocular redness due to allergic reaction to the medical components or preservatives, and it still has the potential causing adverse effects of tachyphylaxis and rebound.

This invention is a fundamentally different approach to the treatment of non-infectious ocular immunoinflammatory diseases, including DED and ocular redness (an independent clinical indication) and does not relate to any current existing therapeutic approach in the treatment of non-infectious ocular immune disorders. Corticosteroids are nonspecific anti-inflammatory agents, and currently used off-label for DED and ocular redness, but they are associated with many untoward side effects. The two FDA approved prescription therapies for DED in the US are topical cyclosporine (RESTASIS®) and lifitegrast (XIIDRAO). RESTASIS® has myriad efficacy and tolerability issues, including ocular burning. XIIDRAO was approved in 2016, and early results are not dramatically different from RESTASIS® with low efficacy as well as many tolerability issues and side effects that lead to patient discontinuation of treatment. Moreover, neither helps ocular redness.

Neurokinin-1 (NK-1) Receptor Antagonists

The neurokinin-1 (NK-1) receptor is a receptor for the neurotransmitter substance P, and is distributed throughout the central nervous system. Certain neurokinin-1 (NK-1) receptor antagonists are known as having antidepressant, anxiolytic, and antiemetic properties. There is currently no evidence demonstrating that any NK1R agonist (such as SP) treatment can reduce DED. In fact, NK1R^{-/-} mice have multiple distinct phenotypes from wild-type mice, including neurologic pathologies. The SP signaling could still be present or even enhanced in this genetically modified mouse strain through those “non-preferred” SP receptors (NK2R or NK3R) in wild-type case (becoming “preferred” in the knockout mouse). Therefore, the precise roles of SP signaling in DED pathogenesis required further studies using better animal models.

Accordingly, in certain embodiments, a composition comprises a therapeutically effective amount of a NK-1 receptor antagonist, a pharmaceutically acceptable salt thereof, a prodrug of the NK-1 receptor antagonist or pharmaceutically acceptable salt thereof, or a solvate or hydrate of the NK-1 compound, of the NK-1 receptor antagonist or of the pharmaceutically acceptable salt thereof.

In certain embodiments, the NK1R antagonist comprises a small molecule antagonist of NK1R, a neutralizing anti-NK1R antibody, a blocking fusion protein against SP, an anti-SP antibody or a nucleic acid. In certain embodiments, the NK1R antagonist is a small molecule.

In certain embodiments, the NK1R antagonist comprises:

### Spantide (RPKPQQWFWLL; SEQ ID NO: 2),

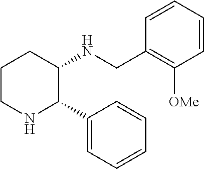 [SMILES: COc1ccccc1CN[C@H]1CCCN[C@H]1c1ccccc1]

(2S,3S)—N-[(2-Methoxyphenyl)methyl]-2-phenyl-3-piperidinamine dihydrochloride,

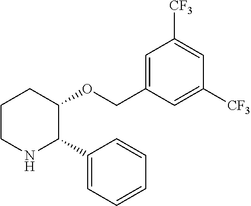 [SMILES: FC(F)(F)c1cc(CO[C@H]2CCCN[C@H]2c2ccccc2)cc(C(F)(F)F)c1]

(2S,3S)-3-[[3,5-bis(Trifluoromethyl)phenyl]methoxy]-2-phenylpiperidine hydrochloride,

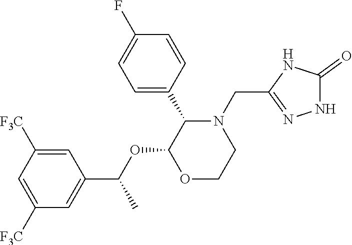 [SMILES: C[C@@H](O[C@H]1OCCN(Cc2n[nH]c(=O)[nH]2)[C@H]1c1ccc(F)cc1)c1cc(C(F)(F)F)cc(C(F)(F)F)c1]

5-[[(2R,3)-2-[(1R)-1-[3,5-Bis(triflromethyl)phenyl]ethoxy]-3-(4-fluorophenyl)-4-morpholinyl]methyl]-1,2-dihydro-3H-1,2,4-triazol-3-one,

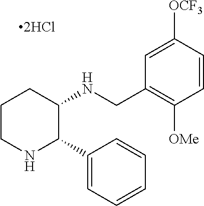 [SMILES: COc1ccc(OC(F)(F)F)cc1CN[C@H]1CCCN[C@H]1c1ccccc1]

(2S,3S)—N-[[2-Methoxy-5-(trifluoromethoxy)phenyl]methyl]-2-phenyl-3-piperidinamine dihydrochloride,

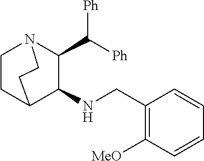 [SMILES: COc1ccccc1CN[C@H]1C2CCN(CC2)[C@H]1C(c1ccccc1)c1ccccc1]

(2S,3S)—N-(2-Methoxyphenyl)methyl-2-diphenylmethyl-1-azabicyclo[2.2.2]octan-3-amine,

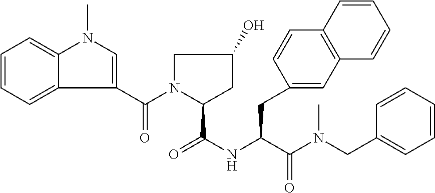 [SMILES: CN(Cc1ccccc1)C(=O)[C@H](Cc1ccc2ccccc2c1)NC(=O)[C@@H]1C[C@@H](O)CN1C(=O)c1cn(C)c2ccccc12]

(4R)-4-Hydroxy-1-[(1-methyl-1H-indol-3-yl)carbonyl]-L-prolyl-N-methyl-3-(2-naphthalenyl-N-(phenylmethyl)-L-alaninamide,

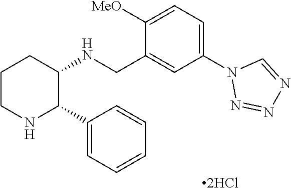 [SMILES: COc1ccc(-n2cnnn2)cc1CN[C@H]1CCCN[C@H]1c1ccccc1]

(2S,3S)—N-[[2-Methoxy-5-(1H-tetrazol-1-yl)phenyl]methyl]-2-phenyl-piperidinamine dihydrochloride,

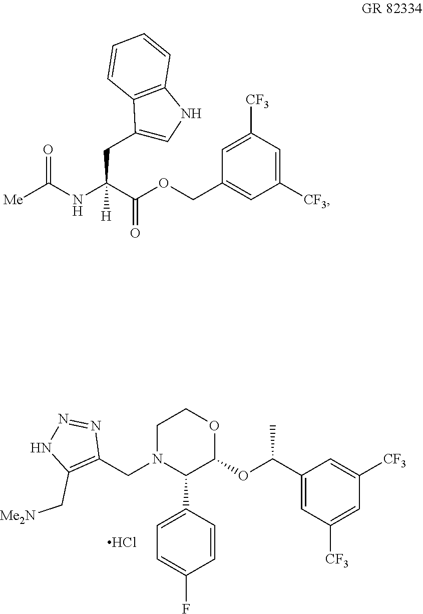

5-[[(2R,3S)-2[(1R)-1-[3,5-Bis(trifluoromethyl)phenyl]ethoxy]-3-(4-fluorophenyl)-4-morpholinyl]methyl-N,N-dimethyl-1H-1,2,3-triazole-4-methanamine hydrochloride,

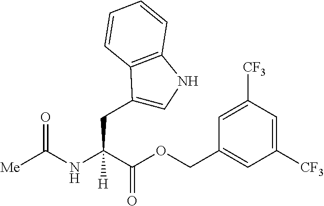

N-Acetyl-L-tryptophan 3,5-bis(trifluoromethyl)benzyl ester,

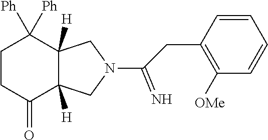 [SMILES: COc1ccccc1CC(=N)N1C[C@@H]2C(=O)CCC(c3ccccc3)(c3ccccc3)[C@@H]2C1]

(3aR,7aR)-Octahydro-2-[1-imino2-(2-methoxyphenyl)ethyl]-7,7-diphenyl-4H-isoindol,

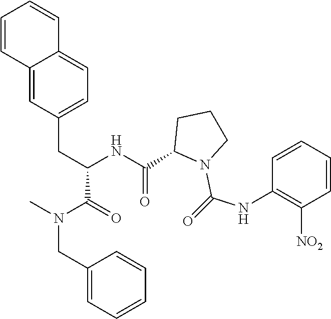 [SMILES: CN(Cc1ccccc1)C(=O)[C@H](Cc1ccc2ccccc2c1)NC(=O)[C@@H]1CCCN1C(=O)Nc1ccccc1[N+](=O)[O-]]

1-[[(2-Nitrophenyl)amino]carbonyl]-L-prolyl-N-methyl-3-2-naphthalenyl)-N-(phenylmethyl)-L-alaninamide

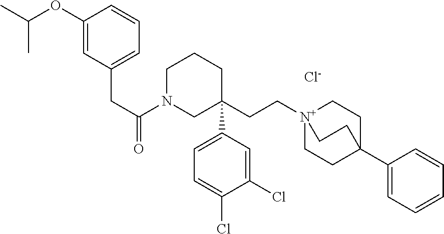 [SMILES: CC(C)Oc1cccc(CC(=O)N2CCC[C@](CC[N+]34CCC(c5ccccc5)(CC3)CC4)(c3ccc(Cl)c(Cl)c3)C2)c1 ; [Cl-]]

1-[2-[(3S)-3-(3,4-Dichlorophenyl)-1-[2-[3-(1-methylthoxy)phenyl]acetyl]-3-piperidinyl]ethyl]-4-phenyl-1-azoniabicyclo[2.2.2]octane chloride, analogs, or combinations thereof.

In certain embodiments, an NK1R antagonist comprises CP-99,994 [(2S,3S)-N-[(2-Methoxyphenyl)methyl]-2-phenyl-3-piperidinamine dihydrochloride] or L-733,060 [(2S,3S)-3-[[3,5-bis(Trifluoromethyl)phenyl]methoxy]-2-phenylpiperidine hydrochloride].

In certain embodiments, the NK1R antagonist comprises L-733,060, L-703,060 or the combination thereof. In certain embodiments, a method of preventing or treating DED and/or ocular redness, comprises administration of to a subject in need thereof, a therapeutically effective amount of L-733,060, L-703,060 or the combination thereof.

In certain embodiments, a pharmaceutical composition comprises an NK antagonist. In certain embodiments, the NK antagonist is selected from achiral pyridine class of neurokinin-1 receptor antagonists; netupitant 21; betctupitant 29; elzlopitant; lanepitant; osanetant; talnetant; GR205171; MK 0517; MK517; MEN 11467; nepadutant; MEN 11420; M274773; [Sar (9), Met (02) (11)]-Substance P; Tyr (6), D-Phe (7), D-His (9)-Substance-P (6-11) (sendide); (beta;-Ala(8))-Neurokinin A (4-10); (Tyr(5), D-Trp (6,8,9), Lys-NH(2) (10))-Neurokinin A; [D-Proz, D-Trip 7,9]-SP DPDT-SP; [D-Proz, D-Phe7, D-Trp9]-SP; SR48968 and 4-Alkylpiperidine derivative; telnetant; SB223412; SB223412A; telnetant hydrochloride; MDL103392; phosphorylated morpholine acetal human neurokinin-1 receptor agonists; SDZ NKT 343; LY 303 870; Ym-35375 and spiro-substituted piperidines; YM-44778; YM-38336; Septide; L732,13; Dactinomyan analogues; MEN 10207; L 659874; L 668,169; FR113680 and derivative; GR 83074; tripeptides possersi, the glutaminyl-D-trypto phy phenyl alonite sequence; L 659,877; R396; Imidazo[4,5-b]quinoxaline cyonines as neurokinin antagonists; MEN 10208; DPDTP-octa; GR73632; GR64349; senktide; GR71251; [D-Argl, D-Pro2, D-Trp 7,9, Leull]-SP (1-11); Ac heu-Asp-Gln-Trp-Phe-Gly NH2; Thr-Asp-Tyr-D-Tvp-Val-D-Trp-D-Trp-Arg NH2; Cyclo [Eln-Trp-Phe-Gly-Leu-Met]; D-Pro2D-Trp 7,9; D-Arg1D-Trp 7,9 leull; [Gly6]-NKB [3-10]; [Arg3, D-Ala6]-NKB [3-10]; CP-9634; 3 aminoquinudidine; CP-99994; S18525; S19752; 4-quinoline carboxinide fremincik class; CP-122721; MK-869; GR205171; Spantide II; CP-96,345; L703,606; SR140, DNK333; 2-phenyl-4-quinolinecarboximides class; FK224; FR 115224; FK888; ZM253270-pyrrolopyrimidine class of nonpeptide neurokinin antagonists; GR71251; GR82334; RP67580; diacylpiperazine antagonists of human neurokinin e.g. L-161664; RP67580; MEN10376; GR98400; N2-[N2-(1H-indol-3-ylcarbonyl)-L-lysyl]-N-methyl-N-(phenyl-methyl)-L-phenylalaninamibe (2b); SP-(1-11); SP-(6-11); SP-(4-11) WIN51703; Spantide II; Spantide III; Spantide I; aprepitant; L754030; MK0869; ONO-7436; ONO 7436; MEN13510; 1-[2-(R)-{1-1R)-[3,5-bis(trifluoromethyl)phenyl]ethoxy}-3-(R)-(3,4-difluorophenyl)-4-(R)-tetrahydro-2H-pyran-4-ylmethyl]-3-(r)-methylpiperdine-3-carboxylic acid (1); LY 306,740; SLV-323; 2-substituted-4-aryl-6,7,8,9-tetrahydro-5H-pyrimido[4,5-b][1,5]oxazocin-5-one; 9-substituted-7-aryl-3,4,5,6-tetrahydro-2H-pyrido[4,3-b]- and [2,3-b]-1,5-oxazocin-6-one; SR142801; SB222200; CP96345; SR48968; ezlopitant; CJ 11974; MEN11558; [18F] SPA-RQ; neuropitant 21; betupitant 29; SR 144190; SR48692; SR141716; L733060; vofopitant; R-673; nepadutant; saredutant; UK 290795; 2-(4-biphenylyl)quinoline-4-carboxylate and carboxamide analogs (neurokinin-3 receptor antagonist); 4-Amino-2-(aryl)-butylbenzamides and analogues; MK-869; L742694; CP 122721; 1-alkyl-5-(3,4-dichlorophenyl)-5-[2-[(3-substituted)-1-azetidinyl]ethyl]-2-piperidines; L760735; L758,298, Cbz-Gly-Leu-Trp-0Bzl(CF(3))(2); L733,061; SR144190; SB235375; N-[(R,R)-(E)-1-arylmethyl-3-(2-oxo-azepan-3-yl) carbamoyl]allyl-N-methyl-3,5-bis(trifluoromethyl)benzamides; 3-[N¹-3,5-bis(trifluoromethyl)benzoyl-N-arylmethyl-N¹-methylhydrazino]-N-[(R)-2-oxo-azepan-3-yl]propionanides; SR142806; SR48,968; CP141,938; LY306740; SB40023; SB414240; Nolpitantium; SR140333; perhydroisoindole RP 67580, Depitant; RPR 100893; Lanepitant; LY-303870; sanoti synthelabo; nolpitanium; SR 140333; SR 48968; Savedutant; AV 608; AV-608, AV608; CGP 60829; NK-608; NKP-608C; NKP608; CS003; R113281; Vestipitant; 597599; GW 597599; GW 597599B; SSR 240600; casopitant; 679769; GW 679769; TA 5538; SSR 146977; SLV317; SLV-317; 823296; GW 823296; AVE 5883; AVE-5883; AZ 311; SB 235375; SB 733210; AZ 685; SAR 102279; SAR 10279; SSR 241586; SLV 332; Neurokinin 2 antagonist-Solvay; SLV-332; SLV332, NIK 616; MPV4505; NIK616; MPC 4505; Z501; Z-501; 1 TAK 637; CP 96345; L 659877; CGP 49823; GR 203040; L 732138; S 16474; WIN 51708; ZD 7944; S 18523; CI 1021; PD 154075; 758298; ZD 4974; S 18920; HMR 2091; FK 355; SCH 205528; NK 5807; NIP 531; SCH 62373; UK 224671; MEN 10627; WIN 64821; MDL 105212A; MEN 10573; TAC 363; 1 MEN 11149; HSP 117; NIP 530; and AZD 5106.

In certain embodiments, the antagonist includes a compound having a formula (I),

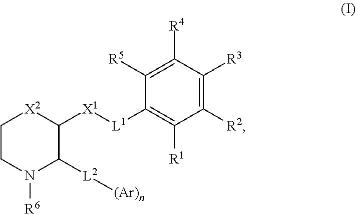

or a pharmaceutically acceptable salt thereof.

Ar is substituted or unsubstituted aryl or heteroaryl.

n is an integer from 1 to 3.

X¹ is —NH—, —C(O)— or —O—.

X² is —CHR⁷— or —O—.

L¹ is a bond, or substituted or unsubstituted alkylene.

L² is a bond, or substituted or unsubstituted alkylene.

Each R¹, R², R³, R⁴, R⁵, R⁶, and R⁷ is independently hydrogen, halogen, substituted or unsubstituted alkylene, substituted or unsubstituted 2 to 4 membered heteroalkyl, substituted or unsubstituted cycloalkyl, substituted or unsubstituted heterocycloalkyl, substituted or unsubstituted aryl, or substituted or unsubstituted heteroaryl; or R⁶ and R⁷ are jointed to form a substituted or unsubstituted heterocycloalkyl.

In certain embodiments, L² is a bond; n is 1; Ar is phenyl; X² is —CH₂—; and R⁶ is hydrogen.

In some embodiments, the antagonist is a compound having a formula (II),

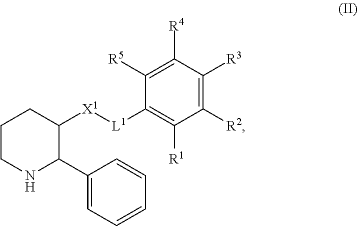

or a pharmaceutically acceptable salt thereof. X¹, L¹, R¹, R², R³, R⁴, and R⁵ are described herein.

In some embodiments, X¹ is —NH— or —O—. In some embodiments, L¹ is substituted or unsubstituted methylene.

In some embodiments, the antagonist has the following formula,

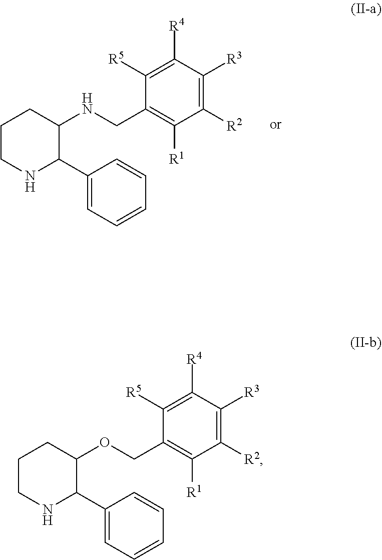

or a pharmaceutically acceptable salt thereof.

In some embodiments, each R¹, R², R⁴ and R⁵ is independently hydrogen, —OCH₃, —OCF₃, —OCH₃, —CF₃, or

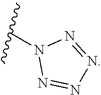 [SMILES: *n1nnnn1]

In some embodiments, R³ is hydrogen.

In some embodiments, R¹ or R⁵ is independently hydrogen or —OCH₃.

In some embodiments, R² or R⁴ is independently hydrogen,

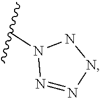

—CF₃ or —OCF₃

In some embodiments, the compound of formula (II-a) includes:

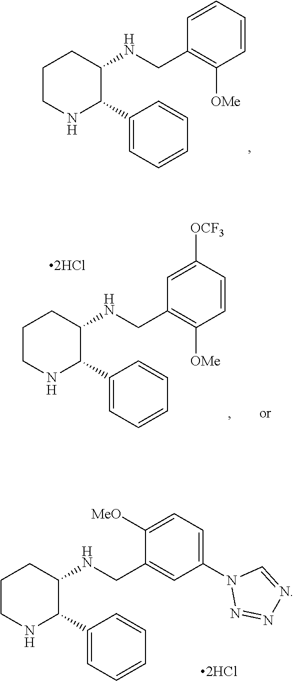 [SMILES: COc1ccccc1CN[C@H]1CCCN[C@H]1c1ccccc1 ; COc1ccc(OC(F)(F)F)cc1CN[C@H]1CCCN[C@H]1c1ccccc1 ; "·2HCl" ; COc1ccc(-n2cnnn2)cc1CN[C@H]1CCCN[C@H]1c1ccccc1 ; "·2HCl"]

In some embodiments, the compound of formula (II-b) includes:

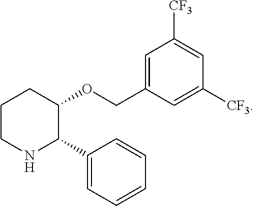 [SMILES: FC(F)(F)c1cc(CO[C@H]2CCCN[C@H]2c2ccccc2)cc(C(F)(F)F)c1]

In certain embodiments, X² is —O—; L² is a bond; and n is 1.

In some embodiments, the antagonist is a compound having a formula (III),

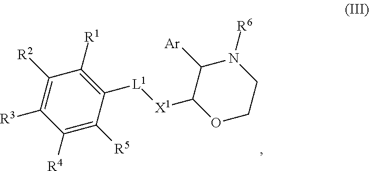

or a pharmaceutically acceptable salt thereof. Ar, X¹, L¹, R¹, R², R³, R⁴, R⁵, and R⁶ are described herein.

In some embodiments, L^{I}- is substituted or unsubstituted methylene. For example, L¹ is —CH(CH₃)—.

In some embodiments, the antagonist is a compound having a formula (III-a).

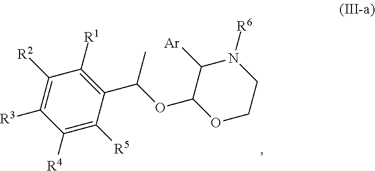

or a pharmaceutically acceptable salt thereof. Ar, R¹, R², R³, R⁴, R⁵, and R⁶ are described herein.

In some embodiments, Ar is substituted or unsubstituted phenyl. In some embodiments, Ar is

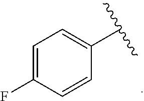 [SMILES: *c1ccc(F)cc1]

In some embodiments, R⁶ is substituted C₁-C₄ alkyl. In some embodiments, R⁶ is

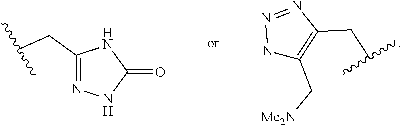 [SMILES: *Cc1n[nH]c(=O)[nH]1 ; *Cc1nn[nH]c1CN(C)C]

In some embodiments, each R¹, R², R⁴ and R⁵ is independently hydrogen, or —CF₃. In some embodiments, R³ is hydrogen.

In some embodiments, each R¹ and R⁵ is independently hydrogen.

In some embodiments, each R² and R⁴ is independently hydrogen or —CF₃.

In some embodiments, the compound of formula (III-a) includes

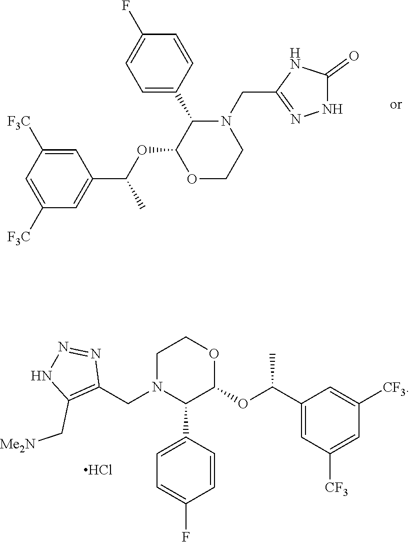 [SMILES: C[C@@H](O[C@H]1OCCN(Cc2n[nH]c(=O)[nH]2)[C@H]1c1ccc(F)cc1)c1cc(C(F)(F)F)cc(C(F)(F)F)c1 ; C[C@@H](O[C@H]1OCCN(Cc2nn[nH]c2CN(C)C)[C@H]1c1ccc(F)cc1)c1cc(C(F)(F)F)cc(C(F)(F)F)c1 ; Cl]

In certain embodiments, R⁶ and R⁷ are joined to form a 5 to 6 membered heterocycloalkyl. In certain embodiments, X² is —CHR⁷—; R⁶ and R⁷ are joined to form a 6 membered heterocycloalkyl; and n is 2.

In some embodiments, the antagonist is a compound having a formula (IV),

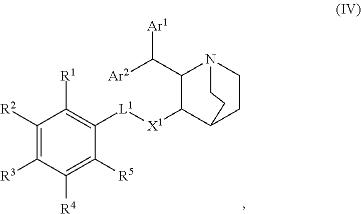

or a pharmaceutically acceptable salt thereof. X¹, L¹, R¹, R², R³, R⁴, and R⁵ are described herein. Ar¹ and Ar² are the same as Ar.

In some embodiments, L² is methylene.

In some embodiments, the antagonist is a compound having a formula (IV-a),

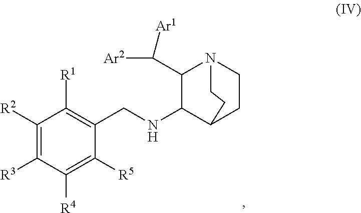

or a pharmaceutically acceptable salt thereof. R¹, R², R³, R⁴, and R⁵ are described herein. Ar¹ and Ar² are the same as Ar.

In some embodiments, Ar¹ and Ar² are phenyl.

In some embodiments, each R¹ and R⁵ is independently hydrogen, or —OCH₃,

In some embodiments, R², R³ and R⁴ are hydrogen.

In some embodiments, the compound of formula (IV) includes

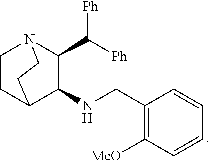 [SMILES: COc1ccccc1CN[C@H]1C2CCN(CC2)[C@H]1C(c1ccccc1)c1ccccc1]

In various embodiments, a composition comprises a polynucleotide, an aptamer, a polypeptide, an antibody or a fragment thereof, or a small molecule that binds or modifies the function of NK1R administered topically with a pharmaceutically appropriate carrier.

Delivery methods for polynucleotide compositions include, but are not limited to, liposomes, receptor-mediated delivery systems, naked DNA, and engineered viral vectors such as herpes viruses, retroviruses, adenoviruses and adeno-associated viruses, among others. Polynucleotide compositions may be administered topically with a pharmaceutically acceptable liquid carrier, e.g., a liquid carrier, which is aqueous or partly aqueous. In certain embodiments, polynucleotide sequences within the composition are associated with a liposome (e.g., a cationic or anionic liposome).

In some embodiments, the NK1R antagonist comprises nucleic acid molecules such as: ribonucleic acids (RNA), deoxyribonucleic acids (DNA), synthetic RNA or DNA sequences, modified RNA or DNA sequences, complementary DNA (cDNA), short guide RNA (sgRNA), a short interfering RNA (siRNA), a micro, interfering RNA (miRNA), a small, temporal RNA (stRNA), a short, hairpin RNA (shRNA), mRNA, nucleic acid sequences comprising one or more modified nucleobases or backbones, or combinations thereof. In certain embodiments, the nucleic acid molecules are antisense oligonucleotides. Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used.

By “antisense oligonucleotides” or “antisense compound” is meant an RNA or DNA molecule that binds to another RNA or DNA (target RNA, DNA). For example, if it is an RNA oligonucleotide it binds to another RNA target by means of RNA-RNA interactions and alters the activity of the target RNA. An antisense oligonucleotide can upregulate or downregulate expression and/or function of a particular polynucleotide. The definition is meant to include any foreign RNA or DNA molecule which is useful from a therapeutic, diagnostic, or other viewpoint. Such molecules include, for example, antisense RNA or DNA molecules, interference RNA (RNAi), micro RNA, decoy RNA molecules, siRNA, enzymatic RNA, short, hairpin RNA (shRNA), therapeutic editing RNA and agonist and antagonist RNA, antisense oligomeric compounds, antisense oligonucleotides, external guide sequence (EGS) oligonucleotides, alternate splicers, primers, probes, and other oligomeric compounds that hybridize to at least a portion of the target nucleic acid. As such, these compounds may be introduced in the form of single-stranded, double-stranded, partially single-stranded, or circular oligomeric compounds.

Antisense oligonucleotide molecules can be directly administered or provided in a DNA construct and introduced into a cell to decrease the level of SP, for example. In certain embodiments, the antisense oligonucleotides specifically bind to regulatory regions resulting in inhibition or enhanced transcription.

In some embodiments, antisense oligonucleotides can be deoxyribonucleotides, ribonucleotides, or a combination of both. In various embodiments, oligonucleotides mag be modified to increase the half-life of the oligonucleotide in vivo. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters.

Modified or Mutated Nucleic Acid Sequences. In some embodiments, any of the nucleic acid sequences embodied herein may be modified or derived from a native nucleic acid sequence, for example, by introduction of mutations, deletions, substitutions, modification of nucleobases, backbones and the like. The nucleic acid sequences include the vectors, gene-editing agents, isolated nucleic acids, antisense oligonucleotides etc. The nucleic acid sequences of the present invention also include variants in which a different base is present at one or more of the nucleotide positions in the compound. For example, if the first nucleotide is an adenosine, variants may be produced which contain thymidine, guanosine or cytidine at this position. This may be done at any of the positions of the isolated nucleic acid sequence. The nucleic acid sequences of the invention may have modifications to the nucleobases or backbones. Examples of some modified nucleic acid sequences envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH₂—NH—O—CH₂, CH, —N(CH₃)—O—CH₂ [known as a methylene(methylimino) or MMI backbone], CH₂—O—N(CH₃)—CH₂, CH₂—N (CH₃)—N(CH₃)—CH₂ and O—N(CH₃)—CH₂—CH₂ backbones, wherein the native phosphodiester backbone is represented as O—P—O—CH,). The amide backbones disclosed by De Mesmaeker et al. Acc. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

The nucleic acid sequences may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp75-77; Gebeyehu, G., et al. Nucl. Acids Res. 1987, 15:4513). A “universal” base known in the art, e.g., inosine may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278).

Another modification of the nucleic acid sequences of the invention involves chemically linking to the nucleic acid sequences one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651).

In certain embodiments, an isolated nucleic acid sequence, comprises combinations of phosphorothioate internucleotide linkages and at least one internucleotide linkage selected from the group consisting of: alkylphosphonate, phosphorodithioate, alkylphosphonothioate, phosphoramidate, carbamate, carbonate, phosphate triester, acetamidate, carboxymethyl ester, and/or combinations thereof. In another preferred embodiment, an isolated nucleic acid sequence optionally comprises at least one modified nucleobase comprising, peptide nucleic acids, locked nucleic acid (LNA) molecules, analogues, derivatives and/or combinations thereof.

It is not necessary for all positions in a given nucleic acid sequence to be uniformly modified, and in fact more than one of the aforementioned modifications may be incorporated in a single nucleic acid sequence or even at within a single nucleoside within a nucleic acid sequence.

Certain isolated nucleic acid sequences are chimeric molecules. “Chimeric molecules” or “chimeras,” in the context of this disclosure, are isolated nucleic acid sequences which contain two or more chemically distinct regions, each made up of at least one nucleotide. These isolated nucleic acid sequences typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense modulation of gene expression. Consequently, comparable results can often be obtained with shorter isolated nucleic acid sequences when chimeric isolated nucleic acid sequences are used, compared to phosphorothioate deoxyoligonucleotides hybridizing to the same target region. Chimeric isolated nucleic acid sequences may be formed as composite structures of two or more oligonucleotides, modified oligonucleotides, oligonucleosides and/or oligonucleotide mimetics.

In another embodiment, the region of the isolated nucleic acid sequence which is modified comprises at least one nucleotide modified at the 2′ position of the sugar, most preferably a 2′-O-alkyl, 2′-O-alkyl-O-alkyl or 2′-fluoro-modified nucleotide. In another embodiment, the isolated nucleic acid sequences can also be modified to enhance nuclease resistance. Cells contain a variety of exo- and endo-nucleases which can degrade nucleic acids. A number of nucleotide and nucleoside modifications have been shown to make nucleic acid sequence into which they are incorporated more resistant to nuclease digestion than the native oligodeoxynucleotide. Nuclease resistance is routinely measured by incubating isolated nucleic acid sequences with cellular extracts or isolated nuclease solutions and measuring the extent of intact oligonucleotide remaining over time, usually by gel electrophoresis. Isolated nucleic acid sequences which have been modified to enhance their nuclease resistance survive intact for a longer time than unmodified isolated nucleic acid sequences. A variety of oligonucleotide modifications have been demonstrated to enhance or confer nuclease resistance. Isolated nucleic acid sequences can contain at least one phosphorothioate modification. In some cases, oligonucleotide modifications which enhance target binding affinity are also, independently, able to enhance nuclease resistance. Some desirable modifications can be found in De Mesmaeker et al. Acc. Chem. Res. 1995, 28:366-374.

In some embodiments, NK1R antagonists comprising RNA molecules, are engineered to comprise one or more modified nucleobases. Modified RNA components include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl- uridine; 5-carboxymethylaminomethyl-2-thiouridine; dihydrouridine; dihydroribosylthymine; 2′-methyladenosine; 2-methyladenosine; N⁶Nmethyladenosine; N⁶, N⁶-dimethyladenosine; N⁶, 2′-O-trimethyladenosine; 2 methylthio-N⁶Nisopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶ threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methylinosine; 1-methyl inosine; 1;2′-O-dimethyl inosine; 2′-O-methyl guanosine; 1-methyl guanosine; N²-methyl guanosine; N², N²-dimethyl guanosine; N², 2′-O-dimethyl guanosine; N², N², 2′-O-trimethyl guanosine; 2′-O-ribosyl guanosine (phosphate); 7-methyl guanosine; N²; 7-dimethyl guanosine; N²; N²; 7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

In other embodiments, RNA modifications include 2′-fluoro, 2¹-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher T. (i.e., higher target binding affinity) than 2′-deoxyoligonucleotides against a given target.

A number of methods have been developed for delivering short DNA or RNA sequences into cells; e.g., polynucleotide molecules can be contacted directly onto the tissue site, or modified polynucleotide molecules, designed to specifically target desired cell types (e.g., sequences linked to peptides or antibodies that specifically bind receptors or antigens expressed on the target cell surface).

An exemplary approach uses a recombinant DNA construct in which the short polynucleotide sequence is placed under the control of a strong polymerase III or polymerase II promoter. The use of such a construct will result in the transcription of sufficient amounts of polynucleotide that will form complementary base pairs with the endogenous transcripts of nucleic acids of the invention and thereby prevent translation of endogenous mRNA transcripts. The invention encompasses the construction of a short polynucleotide using the complementary strand as a template. For example, a vector can be introduced in vivo such that it is taken up by a cell and directs the transcription of an interfering RNA or precursor to a double stranded RNA molecule. Alternatively, the template for the short polynucleotide transcript is placed under the transcriptional control of a cell-type specific promoter or other regulatory element. Thus, diffusion or absorption of a topically administered composition beyond the intended ocular target tissue does not cause deleterious or systemic side effects. The vector remains episomal or becomes chromosomally integrated, as long as it can be transcribed to produce the desired polynucleotide.

Expression vectors are constructed by recombinant DNA technology methods standard in the art. Vectors can be plasmid, viral, or others known in the art, used for replication and expression in mammalian cells. Expression of the sequence encoding the short polynucleotide can be placed under the control of any promoter known in the art to act in mammalian, preferably human cells. Promoters are inducible or constitutive. Exemplary promoters include, but are not limited to: the SV40 early promoter region (Bernoist et al., Nature 290:304, 1981); the promoter contained in the 3′ long terminal repeat of Rous sarcoma virus (Yamamoto et al., Cell, 22:787-797, 1988); the herpes thymidine kinase promoter (Wagner et al., Proc. Natl. Acad. Sci. USA, 78:1441, 1981); or the regulatory sequences of the metallothionein gene (Brinster et al., Nature, 296:39, 1988).

In some embodiments, polypeptide compositions are associated with liposomes alone or in combination with receptor-mediated delivery systems, to enable transport across the plasma membrane. Polypeptide compositions may be, e.g., soluble or membrane-bound. An exemplary receptor-mediated delivery system involves fusion of a low-density or very-low-density lipoprotein containing particle or vesicle to the low-density lipoprotein (LDL) receptor (LDLR) as observed with Hepatitis C Virus (HCV) infection and HCV-mediated drug delivery methods.

In certain embodiments, a composition comprises one or more extracellular or intracellular antibodies (also called intrabodies) raised or directed against NK1R and/or SP (or a subunit thereof). Extracellular antibodies are topically administered with a pharmacologically appropriate aqueous or non-aqueous carrier. Sequences encoding intracellular antibodies are subcloned into a viral or mammalian expression vector, packed in a lipophilic device to facilitate transport across the plasma membrane, and topically administered to eye tissue with a pharmacologically appropriate aqueous or non-aqueous carrier. Once inside the plasma membrane, host cell machinery transcribes, translates, and processes the intrabody code to generate an intracellular function-blocking antibody targeted against NK1R and/or SP (or a subunit thereof). In the case of secreted molecules, intracellular antibodies prevent post-translational modification or secretion of the target protein. In the case of membrane-bound molecules, intracellular antibodies may also prevent intracellular signaling events upon receptor engagement or binding by SP.

In some embodiments, a composition comprises an NK1R antagonist or SP inhibitor wherein the inhibitor inhibits the transcription, transcript stability, translation, modification, localization, secretion, or receptor binding of SP.

### Methods of Treatment

Various embodiments relate to a method for treating an ocular immunoinflammatory disorder by inhibiting antigen-presenting cell maturation and T_{H}17 cell activation and SP-mediated Treg decrease in cell numbers and/or function in an eye tissue. In non-limiting examples, immunoinflammatory disorder comprises ocular redness, DED, autoimmune uveitis, keratoneuralgia, corneal hyperalgesia, corneal alodynia or ocular graft versus host disease. In some embodiments, the method comprises topically administering a compound that preferentially inhibits SP-NK1R signaling.

In certain embodiments, a method of treating a non-infectious ocular immunoinflammatory disorder in a subject, comprising administering to the subject with a regulatory T cell (Treg)-associated ocular disorder a composition comprising a therapeutically effective amount of one or more neurokinin 1 receptor (NK1R) antagonists.

Among the sub-types and subpopulations of T cells and/or of CD4⁺ and/or of CD8⁺ T cells are naïve T (TN) cells, effector T cells (T_{EFF}), memory T cells and sub-types thereof, such as stem cell memory T (T_{SCMX}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MATT) cells, naturally occurring and adaptive regulatory T (Treg) cells, helper T cells, such as T_{H}1 cells, T_{H}2 cells, T_{H}3 cells, T_{H}17 cells, T_{H}9 cells, T_{H}22 cells, follicular helper T cells, alpha/beta T cells, and delta/gamma T cells.

In general, T regulatory cells have been identified as a CD4⁺CD25+T cell population capable of suppressing an immune response. The identification of Foxp3 as a “master-regulator” of Tregs helped define Tregs as a distinct T cell lineage. The identification of additional antigenic markers on the surface of Tregs has enabled identification and FACS sorting of viable Tregs to greater purity, resulting in a more highly-enriched and suppressive Treg population. In addition to CD4 and CD25, both mouse and human Tregs express GITR/AITR, CTLA-4, and express low levels of CD127 (IL-7Ra). Moreover, Tregs can exist in different states which can be identified based on their expression of surface markers. Tregs which develop in the thymus from CD4+thymocytes are known as “natural” Tregs; however, Tregs can also be induced in the periphery from naïve CD4+T cells in response to low-dose engagement of the TCR, TGF beta and IL-2. These “induced” Tregs secrete the immunosuppressive cytokine IL-10. The phenotype of Tregs changes again as they become activated, and markers including GARP in mouse and human, CD45RA in human, and CD103 in mouse have been shown to be useful for the identification of activated Tregs.

There is increasing evidence that Tregs acquire their function through a myriad of mechanisms that may include the secretion of immunosuppressive soluble factors such as IL-9, IL-10 and TGF beta, cell contact mediated regulation via the high affinity TCR and other costimulatory molecules such as CTLA-4, GITR, and cytolytic activity. Under the influence of TGF beta, adaptive Treg cells mature in peripheral sites, including mucosa-associated lymphoid tissue (MALT), from CD4⁺ Treg precursors, where they acquire the expression of markers typical of Tregs, including CD25, CTLA4 and GITR/AITR. Upon up-regulation of the transcription factor Foxp3, Treg cells begin their suppressive effect. This includes the secretion of cytokines including IL-10 and TGF beta which may induce cell-cycle arrest or apoptosis in effector T cells, and blocking co-stimulation and maturation of dendritic cells.

In the examples section which follow, it was demonstrated that immune quiescence was achieved by restoring or enhancing suppressive functions of Tregs in non-infectious ocular immunoinflammatory disorders including DED, via blocking SP signaling. It was found that blockade of SP-NK1R signaling restored or enhanced Treg functions, and thus suppressed inflammation and achieved immune quiescence in ocular immunoinflammatory disorders, such as ocular redness, dry eye disease, and ocular pain. It was also demonstrated that neurokinin-1 receptor antagonism ameliorates dry eye disease by inhibiting antigen-presenting cell maturation and T_{H}17 cell activation.

Aspects of the present subject matter provide a method of reducing T_{H}17 cell abundance in an ocular, adnexal, or lymph tissue of a subject in need thereof including administering to the subject a composition comprising an NK1R antagonist.

Accordingly, in certain embodiments, a method of treating a non-infectious ocular immunoinflammatory disorder in a subject, e.g., ocular redness and/or DED, comprises administering to the subject, a composition comprising a therapeutically effective amount of an NK1R antagonist wherein the NK1R antagonist inhibits antigen-presenting cell maturation and T_{H}17 cell activation.

In certain embodiments, a method of treating a non-infectious ocular immunoinflammatory disorder in a subject, comprises administering to the subject with a regulatory T cell (Treg)-associated ocular disorder a composition comprising a therapeutically effective amount of one or more neurokinin 1 receptor (NK1R) antagonists. In certain embodiments, the Treg-associated ocular disorder is one selected from non- Dry Eye Disease (DED)-related ocular redness, Dry Eye Disease (DED), allergic conjunctivitis and ocular pain. In certain embodiments, the non-DED-related ocular redness comprises allergic ocular redness. In certain embodiments, the non-DED-related ocular redness comprises non-allergic ocular redness.

In certain embodiments, a method of modulating regulatory T (Treg) cell activity or function comprises administering to a subject in need thereof, a pharmaceutical composition comprising a therapeutically effective amount of one or more neurokinin 1 receptor (NK1R) antagonists.

In certain embodiments, a method of reducing a symptom of a non-infectious ocular immunoinflammatory disorder in a subject, comprises administering to the subject with a Treg-associated ocular disorder a composition comprising a therapeutically effective amount of an SP signaling blockade-inducing agent. In certain embodiments, the Treg-associated ocular disorder comprises non-DED-related ocular redness, Dry Eye Disease (DED), allergic conjunctivitis, ocular pain, keratoneuralgia, corneal hyperalgesia, corneal alodynia. Keratoneuralgia has recently generated significant interest amongst both clinicians and scientists due to increasing awareness, and patients suffering with unexplained ocular surface pain and symptoms. This condition is frequently associated with dry eye disease since sensations of dryness and burning in the eye are a common symptom of both neuropathic eye pain and dry eye, but ocular neuropathic pain should be considered as a disease in its own right. Neuropathic pain patients may have few or no signs of aqueous dry eye, and frequently respond poorly to conventional dry eye treatments. Unlike conventional dry eye disease, there may be little or no sign of ocular surface damage, (the condition is sometimes referred to as “pain without stain”), however patients may also have symptoms of dry eye but with pain symptoms that are out of proportion to the dry eye presentation.

The experience of painful sensations in this condition can vary widely, reflecting a variety of causal factors such as: types of noxious stimuli causing insult to ocular surface nociceptors, the types of corneal sensory receptors affected, (including cold-sensing thermoreceptors, mechanoreceptors, and polymodal receptors), the extent of the inflammatory responses, and the type or types of disorders and damage affecting the nervous system.

Accordingly, in in certain embodiments, a method of treating keratoneuralgia, corneal hyperalgesia, corneal alodynia or reducing a symptom thereof, in a subject, comprises administering to the subject a composition comprising a therapeutically effective amount of one or more neurokinin 1 receptor (NK1R) antagonists.

In certain embodiments, one or more secondary agents can be co-administered or administered in conjunction with other therapeutic approaches in the treatment or symptoms thereof, of keratoneuralgia. For example, a secondary agent can be an anti-inflammatory agent such as, topical corticosteroids, topical and oral azithromycin, oral doxycycline, cyclosporine, tacrolimus, anakinra. Other treatment approaches include regenerative therapy, such as, for example, autologous serum eye drops (20-100%), nerve growth factor, platelet rich plasma, umbilical cord serum eye drops. Systemic pharmacotherapy for pain is another therapeutic approach that can be combined with the compositions embodied herein. For example, nortriptyline, amitryptilline, carbamazepine, 3. GABAergic drugs (gabapentin, pregabalin), SNRI like duloxetine and venlafaxine, opioids like Tramadol, Class 1B sodium channel blocker Mexiletine.

In certain embodiments, the NK1R antagonist is administered in combination with a second therapeutic agent or treatment. The NK1R antagonist is administered either simultaneously or sequentially with a secondary composition comprising one or more of the following: an antibiotic, an immunosuppressive composition, an anti-inflammatory composition, a growth factor, a steroid, a chemokine, or a chemokine receptor.

In certain embodiments, the composition comprises one or more antibiotic compositions to be used in combination with an NK1R antagonist. The antibiotic and NK1R antagonist compositions are administered simultaneously or sequentially. Exemplary antibiotic compositions used for combination-therapy with NK1R antagonist include but are not limited to, amikacin, gentamicin, kanamycin, neomycin, netilmicin, streptomycin, tobramycin, teicoplanin, vancomycin, azithromycin, clarithromycin, clarithromycin, dirithromycin, erythromycin, roxithromycin, troleandomycin, amoxicillin, ampicillin, azlocillin, carbenicillin, clozacillin, dicloxacillin, flucozacillin, mezlocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, oflazacin, trovafloxacin, mafenide, sulfacetamide, sulfamethizole, sulfasalazine, sulfisoxazole, tetracycline, trimethoprim, cotrimoxazole, demeclocycline, soxycycline, minocycline, doxycycline, oxytetracycline, or tetracycline.

In some embodiments, the composition comprises an NK1R antagonist, administered simultaneously or sequentially with a second immunosuppressive composition. The composition may be administered, e.g., topically or intraocularly. The second immunosuppressive composition may be administered topically, intraocularly, or systemically. In various embodiments, the immunosuppressive compound may comprise cyclosporin A or an analog thereof a concentration of 0.05-4.0% (mg/ml). Alternatively, or in addition, the immunosuppressive composition may comprise a glucocorticoid, a cytostatic agent, an alkylating agent (nitrogen mustards/cyclophosphamide, nitrosoureas, platinum compounds), an antimetabolic agent (methotrexate, any folic acid analog, azathioprine, mercaptopurine, any purine analog, any pyrimidine analog, any inhibitor of protein synthesis), a cytotoxic antibiotic (dactinomycin, an anthracycline, mitomycin C, bleomycin, mithramycin), a polyclonal antibody (ATGAM®, THYMPGLOBULINE®, any antibody against the antilymphocyte or antithymocyte antigens), a monoclonal antibody (OKT3®, any antibody against the T-cell receptor, any antibody against IL-2, basiliximab/SIMULECt®, declizumab/ZENAPAX®), Tacrolimus/PROGRAF™/FK506, Sirolimus/RAPAMUNE™/Rapamycin, interferon beta, interferon gamma, an opioid, a TNFα binding protein, mycophenolate, or FTY720.

### Pharmaceutical Formulations and Delivery to the Eye

Dosages, formulations, dosage volumes, regimens, and methods for antagonizing the NK1R can vary. Thus, minimum and maximum effective dosages vary depending on the method of administration. In certain embodiments, an NK1R antagonist is formulated as a topical formulation. In certain embodiments, the topical formulation is a liquid drop. In certain embodiments, the liquid drop comprises at least 0.0001 μg/μl to about 50 μg/μl of one or more NK1R antagonists. In certain embodiments, the liquid drop comprises at least 0.001 μg/μl to about 50 μg/82 l of one or more NK1R antagonists. In certain embodiments, the liquid drop comprises at least 0.01 μg/μl to about 50 μg/μl of one or more NK1R antagonists. In certain embodiments, the liquid drop comprises at least 0.1 μg/μl to about 50 μg/μl of one or more NK1R antagonists. In certain embodiments, the liquid drop comprises at least 0.0001 μg/μl to about 40 μg/μl of one or more NK1R antagonists. In certain embodiments, the liquid drop comprises at least 0.0001 μg/μl to about 35 μg/μl of one or more NK1R antagonists. In certain embodiments, the liquid drop comprises at least 0.0001 μg/μl to about 30 μg/μl of one or more NK1R antagonists. In certain embodiments, the liquid drop comprises at least 0.0001 μg/μl to about 25 μg/μl of one or more NK1R antagonists. In certain embodiments, the liquid drop comprises at least 0.1μg/μl to about 10 μg/μl of one or more NK1R antagonists.

In certain embodiments, the liquid drops comprising the NK1R antagonist(s) are administered topically to each eye at least once a day up to 4 or 5 times a day. In certain embodiments, the liquid drops comprising the NK1R antagonist(s) are administered for a duration of at least one to two or more days for ocular redness or as needed and indefinitely for dry eye therapy.

In various embodiments of the invention, a composition comprising an NK1R antagonist may be administered only once or multiple times. For example, an NK1R antagonist may be administered using a method disclosed herein at least about once, twice, three times, four times, five times, six times, or seven times per day week, month, or year. In some embodiments, a composition comprising an NK1R antagonist is administered once per month. In certain embodiments, the composition is administered once per month via intravitreal injection. In various embodiments, such as embodiments involving eye drops, a composition is self-administered.

In certain embodiments, the liquid eye drops are formulated in a pharmaceutically acceptable inactive excipient or carrier such as phosphate buffered saline and stored at 4° C.

Preferred formulations are in the form of a solid, a paste, an ointment, a gel, a liquid, an aerosol, a mist, a polymer, a contact lens, a film, an emulsion, or a suspension. The formulations are administered topically, e.g., the composition is delivered and directly contacts the eye. The composition is present at a concentration of 0.01-50% (weight/volume). For example, the inhibitory composition is present at concentrations of 1% (weight/volume), 10% (weight/volume), 20% (weight/volume), 25% (weight/volume), 30% (weight/volume), 40% (weight/volume), 50% (weight/volume), or any percentage point in between. The method does not involve systemic administration or planned substantial dissemination of the composition to non-ocular tissue.

Optionally, the composition further contains a pharmaceutically-acceptable carrier. Exemplary pharmaceutical carriers include, but are not limited to, compounds selected from the group consisting of a physiological acceptable salt, poloxamer analogs with carbopol, carbopol/hydroxypropyl methyl cellulose (HPMC), carbopol-methyl cellulose, a mucolytic agent, carboxymethylcellulose (CMC), hyaluronic acid, cyclodextrin, and petroleum. In one embodiment, the mucolytic agent is N-acetyl cysteine.

For the treatment of an ocular immunoinflammatory disease, an NK1R antagonist (e.g., a pharmaceutical composition comprising an NK1R antagonist) may be administered locally, e.g., as a topical eye drop, peri-ocular injection (e.g., sub-tenon), intraocular injection, intravitreal injection, retrobulbar injection, intraretinal injection, subconjunctival injection, or using iontophoresis, or peri-ocular devices which can actively or passively deliver drug.

Pharmaceutical formulations adapted for topical administration may be formulated as aqueous solutions, ointments, creams, suspensions, lotions, powders, solutions, pastes, gels, sprays, aerosols, liposomes, microcapsules, microspheres, or oils.

Pharmaceutical formulations adapted for topical administrations to the eye include eye drops wherein an NK1R antagonist is dissolved or suspended in a suitable carrier, especially an aqueous solvent. Formulations to be administered to the eye will have ophthalmically compatible pH and osmolality. The term “ophthalmically acceptable vehicle” means a pharmaceutical composition having physical properties (e.g., pH and/or osmolality) that are physiologically compatible with ophthalmic tissues.

In some embodiments, an ophthalmic composition of the present invention is formulated as sterile aqueous solutions having an osmolality of from about 200 to about 400 milliosmoles/kilogram water (“mOsm/kg”) and a physiologically compatible pH. The osmolality of the solutions may be adjusted by means of conventional agents, such as inorganic salts (e.g., NaCl), organic salts (e.g., sodium citrate), polyhydric alcohols (e.g., propylene glycol or sorbitol) or combinations thereof.

In various embodiments, the ophthalmic formulations of the present invention may be in the form of liquid, solid or semisolid dosage form. The ophthalmic formulations of the present invention may comprise, depending on the final dosage form, suitable ophthalmically acceptable excipients. In some embodiments, the ophthalmic formulations are formulated to maintain a physiologically tolerable pH range. In certain embodiments, the pH range of the ophthalmic formulation is in the range of from about 5 to about 9. In some embodiments, pH range of the ophthalmic formulation is in the range of from about 6 to about 8, or is about 6.5, about 7, or about 7.5.

In some embodiments, the composition is in the form of an aqueous solution, such as one that can be presented in the form of eye drops. By means of a suitable dispenser, a desired dosage of the active agent can be metered by administration of a known number of drops into the eye, such as by one, two, three, four, or five drops.

One or more ophthalmically acceptable pH adjusting agents and/or buffering agents can be included in a composition of the invention, including acids such as acetic, boric, citric, lactic, phosphoric, and hydrochloric acids; bases such as sodium hydroxide, sodium phosphate, sodium borate, sodium citrate, sodium acetate, and sodium lactate; and buffers such as citrate/dextrose, sodium bicarbonate, and ammonium chloride. Such acids, bases, and buffers can be included in an amount required to maintain pH of the composition in an ophthalmically acceptable range. One or more ophthalmically acceptable salts can be included in the composition in an amount sufficient to bring osmolality of the composition into an ophthalmically acceptable range. Such salts include those having sodium, potassium, or ammonium cations and chloride, citrate, ascorbate, borate, phosphate, bicarbonate, sulfate, thiosulfate, or bisulfite anions.

Pharmaceutical compositions for ocular delivery also include in situ gellable aqueous composition. Such a composition comprises a gelling agent in a concentration effective to promote gelling upon contact with the eye or with lacrimal fluid. Suitable gelling agents include but are not limited to thermosetting polymers. The term “in situ gellable” as used herein includes not only liquids of low viscosity that form gels upon contact with the eye or with lacrimal fluid, but also includes more viscous liquids such as semi-fluid and thixotropic gels that exhibit substantially increased viscosity or gel stiffness upon administration to the eye. See, for example, Ludwig, Adv. Drug Deliv. Rev. 3; 57:1595-639 (2005), the entire content of which is incorporated herein by reference.

### Drug Delivery by Contact Lens

A contact lens and a composition comprising an NK1R antagonist are provided herein. For example, the composition is incorporated into or coated onto the lens. The composition is chemically bound or physically entrapped by the contact lens polymer. Alternatively, a color additive is chemically bound or physically entrapped by the polymer composition that is released at the same rate as the therapeutic drug composition, such that changes in the intensity of the color additive indicate changes in the amount or dose of therapeutic drug composition remaining bound or entrapped within the polymer. Alternatively, or in addition, an ultraviolet (UV) absorber is chemically bound or physically entrapped within the contact lens polymer. The contact lens is either hydrophobic or hydrophilic.

Exemplary materials used to fabricate a hydrophobic lens with means to deliver the compositions of the invention include, but are not limited to, amefocon A, amsilfocon A, aquilafocon A, arfocon A, cabufocon A, cabufocon B, carbosilfocon A, crilfocon A, crilfocon B, dimefocon A, enflufocon A, enflofocon B, erifocon A, flurofocon A, flusilfocon A, flusilfocon B, flusilfocon C, flusilfocon D, flusilfocon E, hexafocon A, hofocon A, hybufocon A, itabisfluorofocon A, itafluorofocon A, itafocon A, itafocon B, kolfocon A, kolfocon B, kolfocon C, kolfocon D, lotifocon A, lotifocon B, lotifocon C, melafocon A, migafocon A, nefocon A, nefocon B, nefocon C, onsifocon A, oprifocon A, oxyfluflocon A, paflufocon B, paflufocon C, paflufocon D, paflufocon E, paflufocon F, pasifocon A, pasifocon B, pasifocon C, pasifocon D, pasifocon E, pemufocon A, porofocon A, porofocon B, roflufocon A, roflufocon B, roflufocon C, roflufocon D, roflufocon E, rosilfocon A, satafocon A, siflufocon A, silafocon A, sterafocon A, sulfocon A, sulfocon B, telafocon A, tisilfocon A, tolofocon A, trifocon A, unifocon A, vinafocon A, and wilofocon A.

Exemplary materials used to fabricate a hydrophilic lens with means to deliver the compositions of the invention include, but are not limited to, abafilcon A, acofilcon A, acofilcon B, acquafilcon A, alofilcon A, alphafilcon A, amfilcon A, astifilcon A, atlafilcon A, balafilcon A, bisfilcon A, bufilcon A, comfilcon A, crofilcon A, cyclofilcon A, darfilcon A, deltafilcon A, deltafilcon B, dimefilcon A, droxfilcon A, elastofilcon A, epsilfilcon A, esterifilcon A, etafilcon A, focofilcon A, galyfilcon A, genfilcon A, govafilcon A, hefilcon A, hefilcon B, hefilcon C, hilafilcon A, hilafilcon B, hioxifilcon A, hioxifilcon B, hioxifilcon C, hydrofilcon A, lenefilcon A, licryfilcon A, licryfilcon B, lidofilcon A, lidofilcon B, lotrafilcon A, lotrafilcon B, mafilcon A, mesafilcon A, methafilcon B, mipafilcon A, nelfilcon A, netrafilcon A, ocufilcon A, ocufilcon B, C, ocufilcon D, ocufilcon E, ofilcon A, omafilcon A, oxyfilcon A, pentafilcon A, perfilcon A, pevafilcon A, phemfilcon A, polymacon, senofilcon A, silafilcon A, siloxyfilcon A, surfilcon A, tefilcon A, tetrafilcon A, trilfilcon A, vifilcon A, vifilcon B, and xylofilcon A.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### EXAMPLES

### Example 1: Increased Level of Substance P (SP) in DED Mouse Model

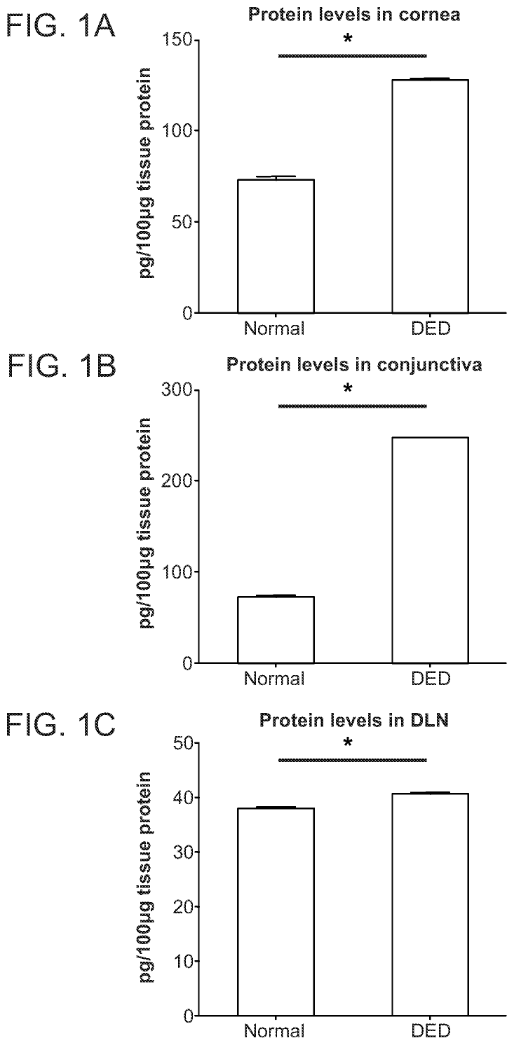

Regulatory T cells (Tregs) are the major component suppressing inflammation and maintaining immune quiescence (Nat Rev Immunol. 2008; 8:523-532). Tregs have been shown to be defective in suppressing inflammation in DED, and restoration of Treg function is crucial to treat DED (J Immunol 2009). In addition, FIGS. 1A-1C show increased levels of SP in DED ocular surface (FIGS. 1A and 1B) and in draining lymph nodes (FIG. 1C), indicating a potentially therapeutic efficacy of blocking SP signaling in DED. In FIGS. 1A-1C, cornea, conjunctiva, and draining lymph nodes (DLN) tissues from DED mice were harvested and homogenized. The level of SP in the tissue homogenates were measured using the ELISA kit. SP protein was significantly increased in DED (*, p<0.05).

However, SP signaling has also been reported to play a critical physiological role in maintaining corneal epithelial homeostasis (PLoS One 2016; 11:e0149865; J. Immunol. 2016; 197:4021-33), and to promote corneal wound healing (Diabetes 2014; 63:4262-74; J Cell Physiol. 1996; 169:159-6). For example, US2017/0246238 claims that activating (instead of blocking) SP signaling mediated eye protection, and can reduce dry eye (US2017/0246238), supported by Suvas S. et al.'s observation that mice genetically deficient in SP receptor NK1R (NK1R^{-/-}) presented DED-associated clinical features. However, there is currently no evidence demonstrating that any NK1R agonist (such as SP) treatment can reduce DED. In fact, NK1R^{-/-} mice have multiple distinct phenotypes from wild-type mice, including neurologic pathologies. The SP signaling could still be present or even enhanced in this genetically modified mouse strain through those “non-preferred” SP receptors (NK2R or NK3R) in wild-type case (becoming “preferred” in the knockout mouse). Therefore, the precise roles of SP signaling in DED pathogenesis required further studies using better animal models.

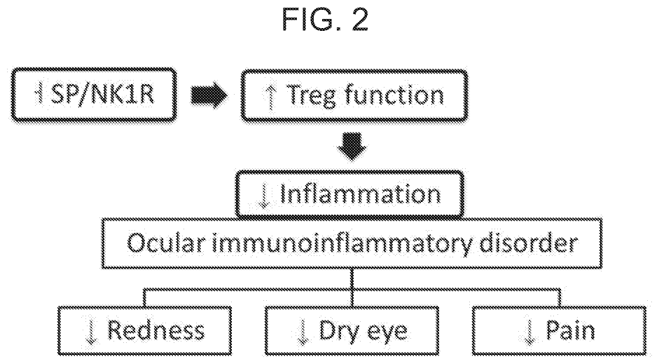

The present application describes achieving immune quiescence by restoring or enhancing suppressive functions of Tregs in non-infectious ocular immunoinflammatory disorders including DED, via blocking SP signaling (FIG. 2). For instance, FIG. 2 illustrates that blockade of SP-NK1R signaling restores or enhances Treg functions, and thus suppresses inflammation and achieve immune quiescence in ocular immunoinflammatory disorders, such as ocular redness, dry eye disease, and ocular pain (−I, blocking; ↑, enhancing; ↓, suppressing or decreasing).

This observation is distinctly the opposite of that of US2017/0246238, as the present application presents that SP antagonism (not agonism) is therapeutic.

### Example 2: NK1R Antagonists Abrogate SP-Mediated Treg Decrease

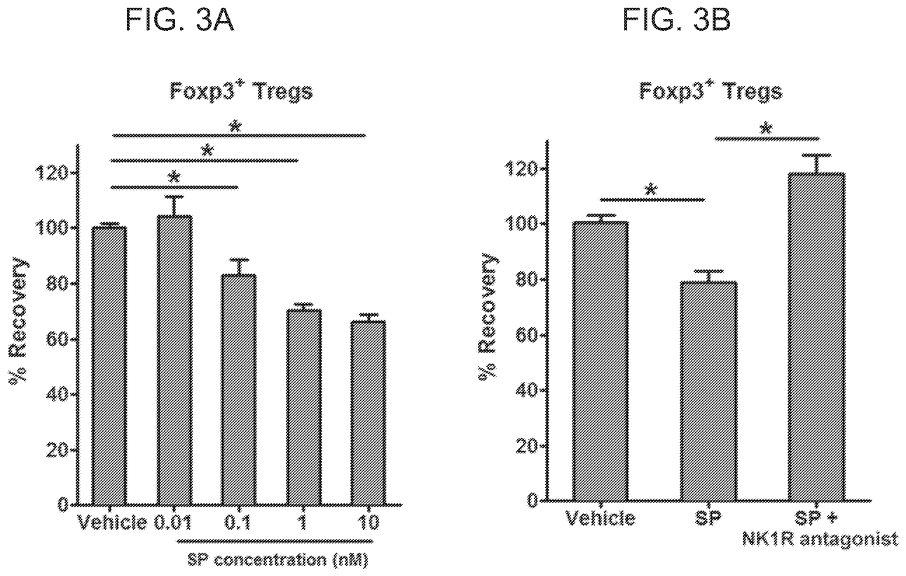

FIGS. 3A and 3B show the results of the in vitro effect of SP on Treg cells. The normal functional Foxp3⁺CD4⁺ Tregs were isolated from naive mice and co-cultured with different doses of SP, e.g., at 0.01, 0.1, 1, and 10 nM in vitro. It was found that starting from 0.1 nM, SP at 0.1, 1, and 10 nM significantly reduced Tregs (FIG. 3A). On the contrary, addition of the NK1R antagonist prevented the loss of Tregs (FIG. 3B). Isolated Tregs were co-cultured with SP (1 nM), and led to a significant decrease in recovered Tregs. When NK1R antagonist spantide I (100 μM) was added to the culture, SP-induced Treg reduction was abrogated (*, p<0.05) (FIG. 3B). These data demonstrate an in vitro protective effect of blocking SP-NK1R signaling on Tregs indicative of clinical therapeutic efficacy.

### Example 3: Topical NK1R Antagonists Reduce DED Severity and Suppress Ocular Surface Inflammation

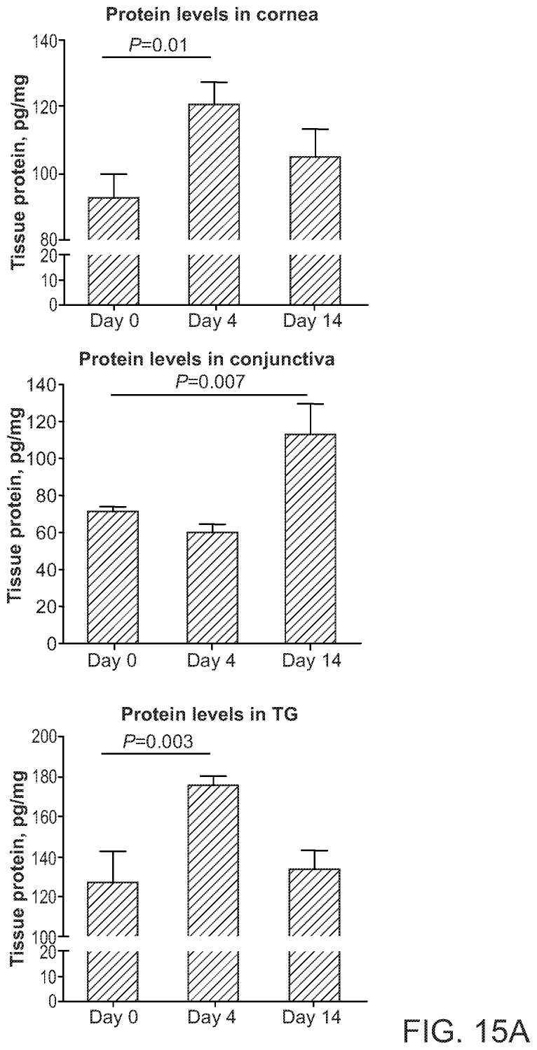

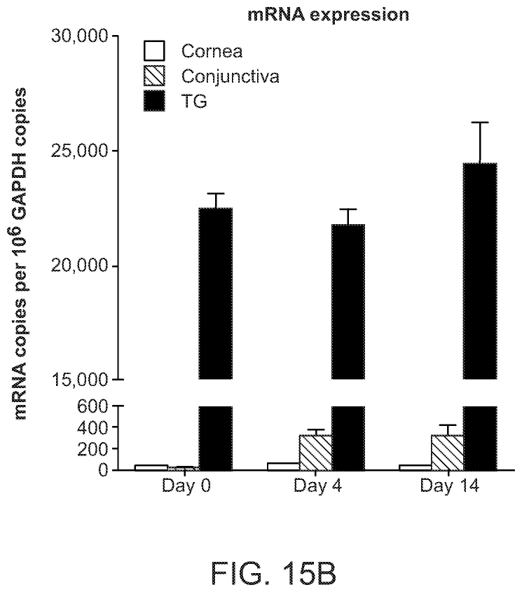

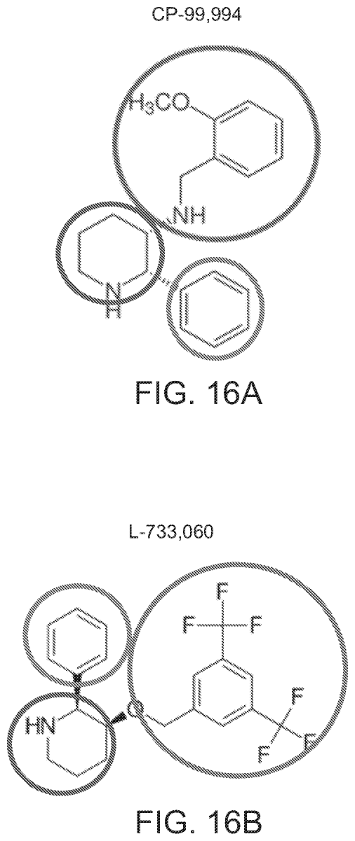

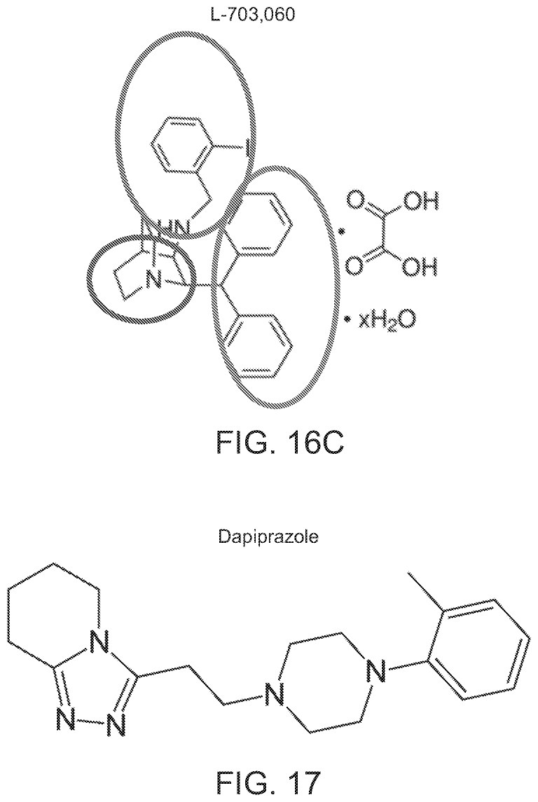

Substance P induces Treg dysfunction by suppressing Treg expression of inhibitory molecules and secreted immunoregulatory cytokines, which is reversed by Spantide I. FIGS. 15A and 15B are the results obtained demonstrating that substance P levels increase in the ocular surface in the course of dry eye disease (DED). DED was induced in C57BL/6 mice for 14 days. Corneas, conjunctiva, and trigeminal ganglia (TGs) of control (day 0) and DED mice (days 4 and 14) were harvested; and tissues were homogenized. FIG. 15A shows the results of studies in which protein levels of substance P in the cornea, conjunctiva, and TG were analyzed in the supernatant of homogenized tissues of control and DED mice using enzyme-linked immunosorbent assay; and protein levels were adjusted to the total protein measured by the BCA Protein assay kit (Thermo Scientific, Rockford, Ill.). FIG. 15B shows the results of studies in which Substance P mRNA levels in the cornea, conjunctiva, and TG of control and DED mice were analyzed using real-time PCR. Data are expressed as means ±SEM (FIGS. 15A and 15B). n=2 independent experiments (FIGS. 15A and 15B).

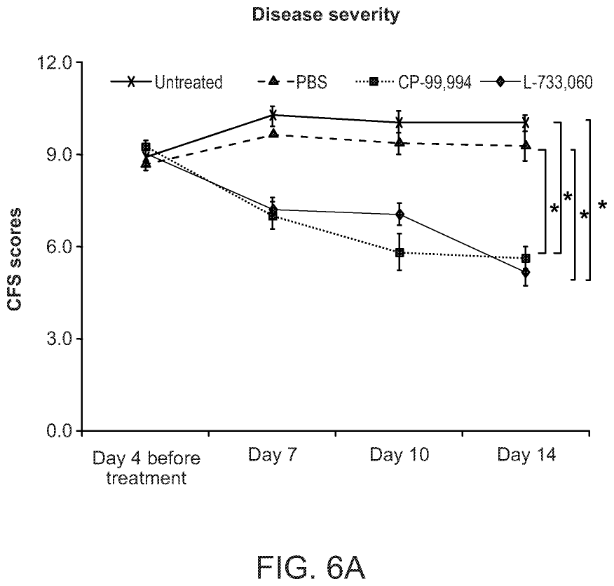

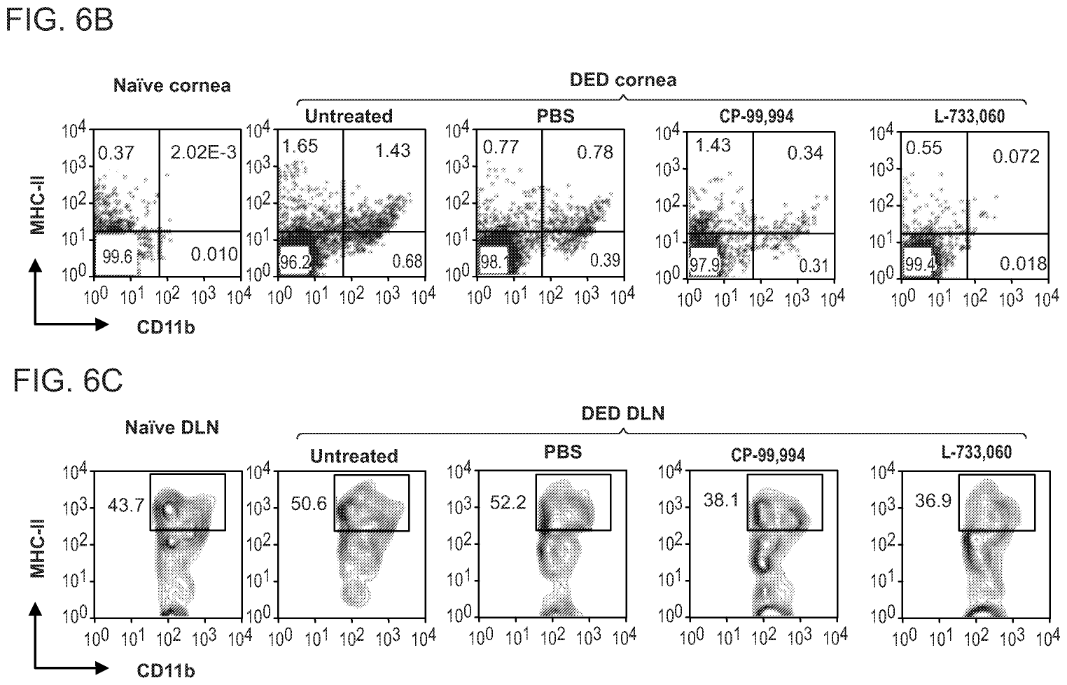

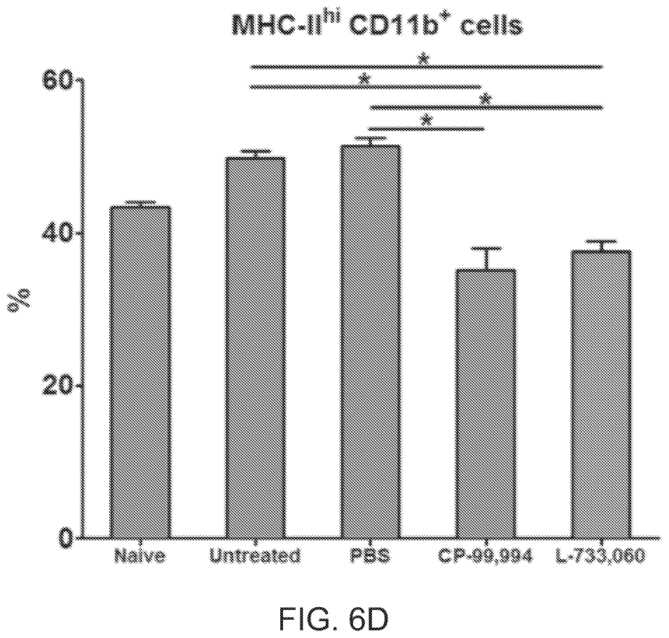

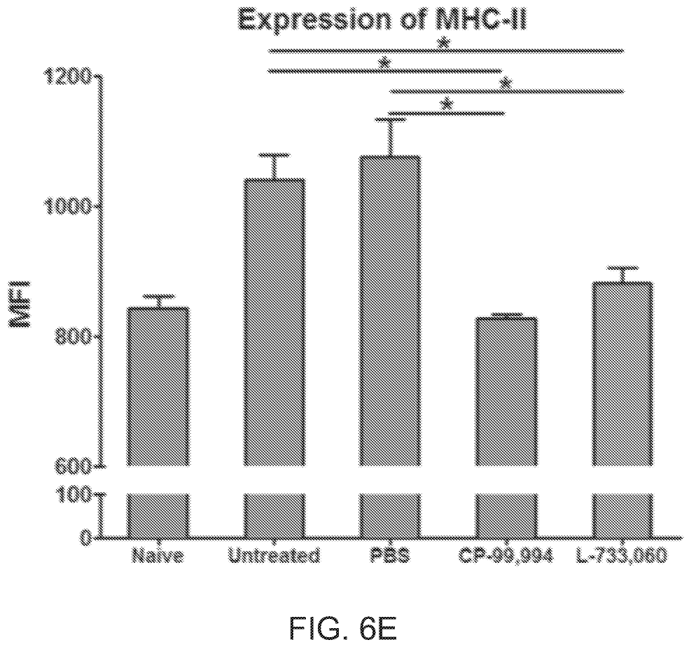

FIGS. 6A, 6B, 7C, 8A-8C, and 9A-9D show the results of evaluating the in vivo therapeutic efficacy of blocking SP signaling in animal models of DED and ocular redness. DED was induced by exposing wild-type mice to desiccating stress using a controlled environment chamber for 14 days. No any other manipulations were used in the model. After 4 days induction, a clinical overt disease developed in mice (art-recognized model) and then a topical NK1R antagonist of either CP-99,994 or L-733,060, 3×/day, started and lasted for 10 days (until day 14). Untreated or PBS-treated mice serve as controls. Clinical severity of the disease was evaluated by clinical fluorescein scoring (CFS) using the National Eye Institute/Industry NEI grading scheme. The NEI scale for grading fluorescein staining divides the corneal and conjunctival surfaces to help measure fluorescein uptake. A standardized grading system of 0 to 3 is used for each of the five areas on each cornea. Grade 0 is specified when no staining is present, and the maximum score is 15.

Topical CP-99,994 or L-733,060 significantly decreased CFS scores in DED mice as compared to untreated or vehicle (e.g., phosphate buffered saline (PBS))-treated mice at day 7, 10, and 14 (p<0.05) (FIG. 6A). In addition, the inflammatory infiltration in the ocular surface was significantly suppressed, e.g., reduced by NK1R antagonism (e.g., topical CP-99,994 or L-733,060), evidenced by significant reduction of activated CD11b⁺ cells (e.g., MHC-II⁺CD11b⁺ cells) in cornea (FIG. 6B) and T_{H}17 cells (e.g., IL-17⁺ CD4⁺ cells) in conjunctiva (FIG. 6C) (*, p<0.05). These data demonstrate that topical blockade of SP receptor NK1R effectively decreased DED severity by restoring ocular surface immune quiescence, indicating that topical NK1R blocker is an efficacious for treatment of ocular immunoinflammatory diseases.

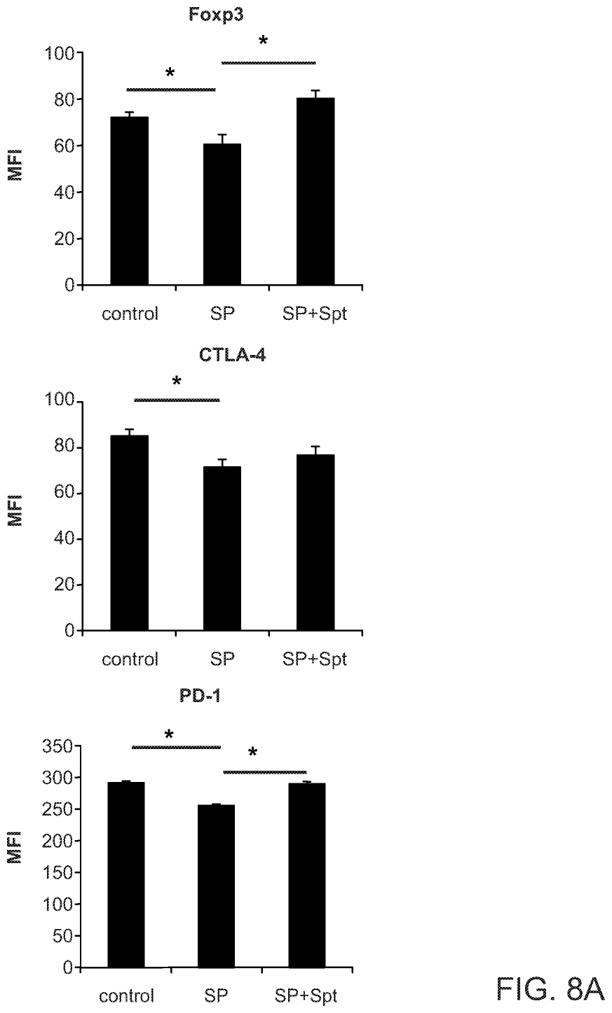

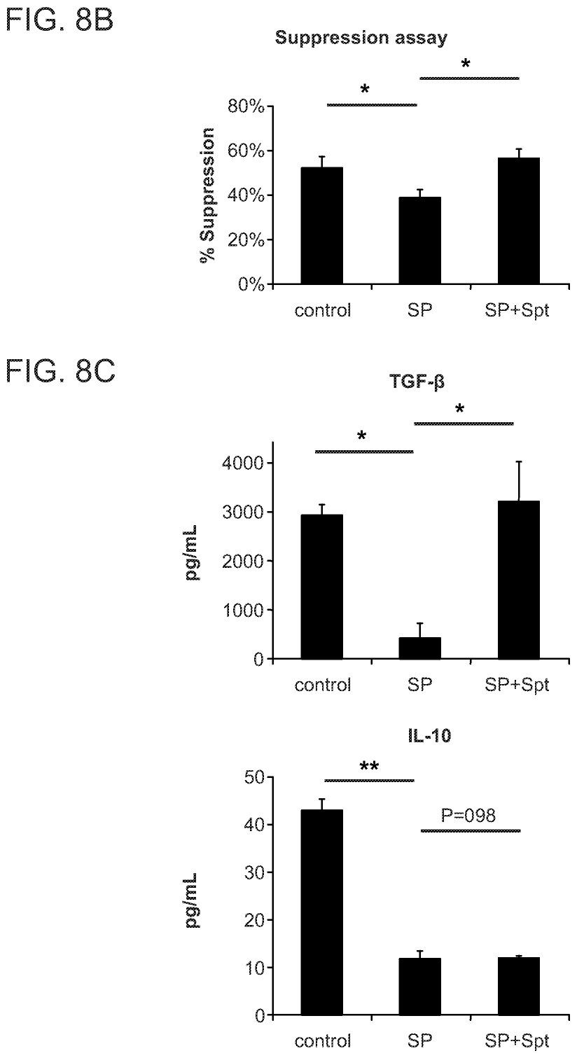

FIG. 8A shows results demonstrating that SP suppresses the expression of inhibitory molecules Foxp3, CTLA4 and PD-1 by Tregs in vitro. In addition, incubation of Tregs with SP results in secretion of significantly lower levels of immunoregulatory cytokines (TGFβ and IL-10) by Tregs (FIG. 8B). FIG. 8C demonstrates a lower capacity of Tregs pre-incubated with SP to suppress effector T cell proliferation in vitro. However, addition of NK-1R antagonist, Spantide I (Spt), to the co-culture reverses SP-induced Treg dysfunction. Data represent mean±SEM. *p<0.05.

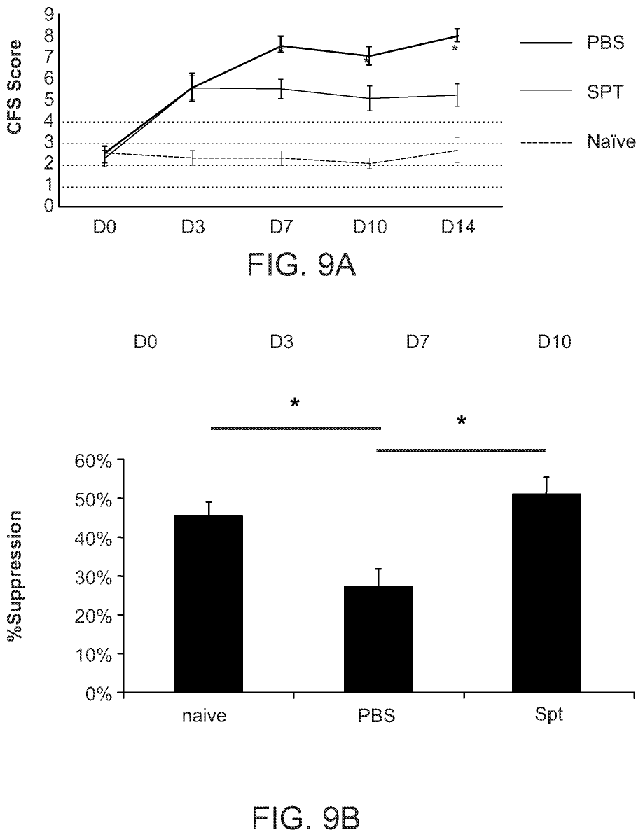

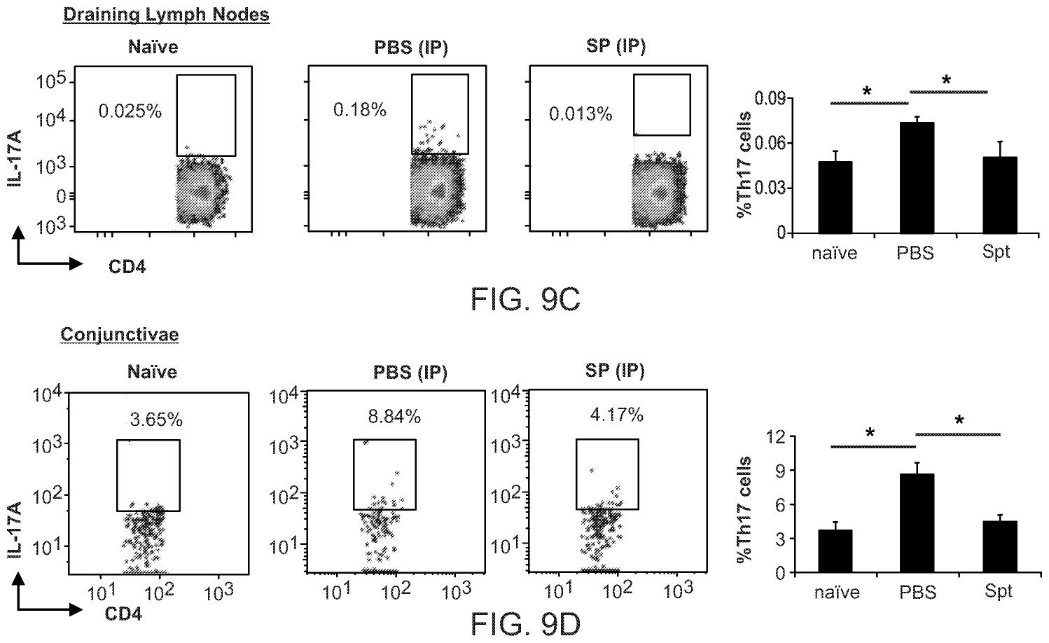

FIGS. 9A-9D show results demonstrating that inhibition of SP signaling through systemic administration of Spantide I restores Treg function, suppresses T_{H}17 cells and reduces the severity of DED. FIG. 9A shows that DED mice treated with Spantide I (Spt) had significantly lower corneal fluorescein staining (CFS) scores compared to PBS-treated controls. FIG. 9B shows that Tregs isolated from DLN of DED mice treated with Spantide I had a higher capacity in suppressing effector T cell proliferation than Tregs derived from the control group. Treatment of DED mice with Spantide I led to a significant reduction in frequencies of CD4⁺IL-17⁺ THh17 cells in (FIG. 9C) draining lymph nodes and (FIG. 9D) conjunctivae of DED mice compared to the control, thus demonstrating efficacy in reducting the severity of DED. Data represent mean±SEM. *p<0.05 **p<0.001.

These data demonstrated that topical blockade of SP receptor NK1R effectively decreased DED severity by restoring ocular surface immune quiescence, indicating that a topical NK1R blocker is an effective therapeutic intervention for ocular immunoinflammatory diseases such as DED and/or ocular redness.

### NK1R: UniProt P25103

(SEQ ID NO: 1)
MDNVLPVDSDLSPNISTNTSEPNQFVQPAWQIVLWAAAYTVIVVTSVVG
NVVVMWIILAHKRMRTVTNYFLVNLAFAEASMAAFNTVVNFTYAVHNEW
YYGLFYCKFHNFFPIAAVFASIYSMTAVAFDRYMAIIHPLQPRLSATAT
KVVICVIWVLALLLAFPQGYYSTTETMPSRVVCMIEWPEHPNKIYEKVY
HICVTVLIYFLPLLVIGYAYTVVGITLWASEIPGDSSDRYHEQVSAKRK
VVKMMIVVVCTFAICWLPFHIFFLLPYINPDLYLKKFIQQVYLAIMWLA
MSSTMYNPIIYCCLNDRFRLGFKHAFRCCPFISAGDYEGLEMKSTRYLQ
TQGSVYKVSRLETTISTVVGAHEEEPEDGPKATPSSLDLTSNCSSRSDS
KTMTESFSFSSNVLS.

### Example 4: NK1R Antagonists Reduce Severity of Ocular Redness

An Image J-based, objective quantification method of ocular redness severity was developed without relying on ophthalmologists' subjective scoring (Amparo, et al. “The Ocular Redness Index: A Novel Automated Method for Measuring Ocular Injection.” Investigative Ophthalmology & Visual Science, 2013, pp. Quick submit: 2017-06-18T21:14:33-0400). This digital scoring method automatically analyzes patient eye images and gives the score as Ocular Redness Index (ORI), which ranges on a continuous centesimal scale from 0 to 100, with 100 the most severe redness. This method has been used in the animal models of ocular redness to evaluate the severity of redness.

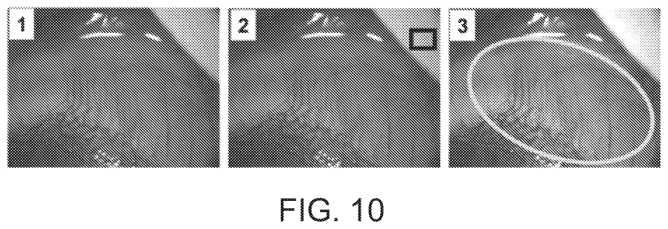

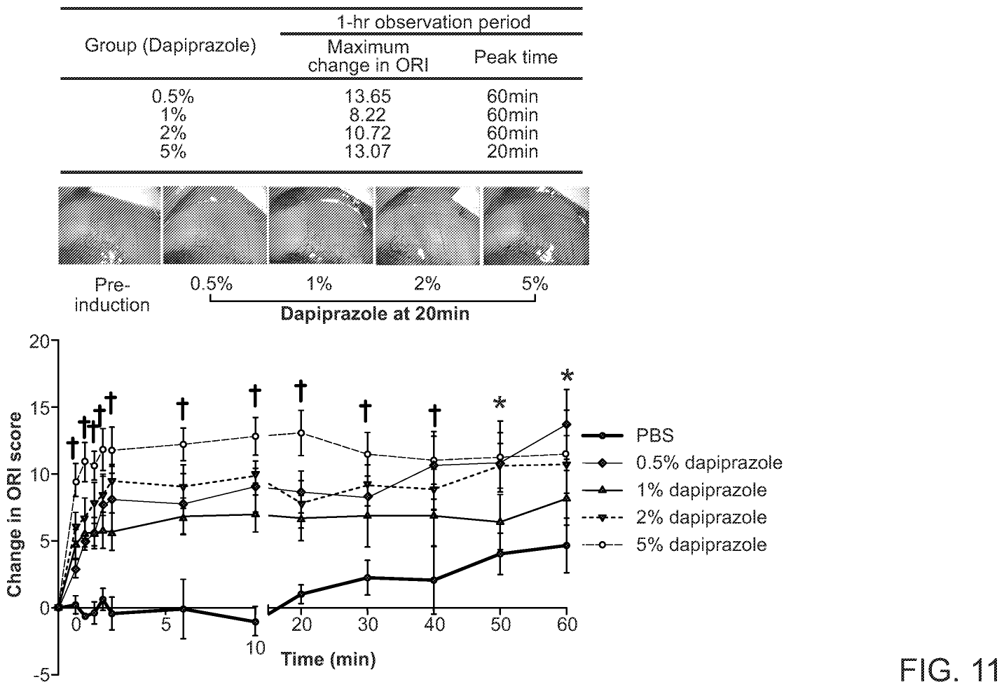

The results obtained can be seen in FIGS. 10-14. In the evaluation of ocular redness in animal models using Ocular Redness Index (ORI) with Image J the following steps. Step 1: The conjunctival image was captured by means of a digital camera and was recorded as an RGB color JPEG image (3264×2448 pixels). Step 2: To reduce the background noise in the images, a color correction (white balance) was made according to selected filter paper area (black box). Step 3: An ROI (Region Of Interest, area inside the yellow circle) was selected as the evaluation area by user, and the program read the red-green-blue (RGB) values of each pixel, converting it to huesaturation-value (HSV) space, and ultimately converted these values into a numeric centesimal value for redness in the selected area automatically.

FIG. 11 shows the results obtained from a rabbit model of non-allergic (non-infectious) ocular redness. Animals were anesthetized, and 40 μl of a series of increasing concentrations of dapiprazole was applied to the right eyes of animals to induce ocular redness. PBS was used as a control on the left eyes. Eyes were examined by a slit lamp, and images were taken pre-induction, and every 30 seconds for the first 2 minutes post-induction, followed by every 4 minutes for additional 8 minutes and every 10 minutes until 1 hour. The table summarizes the maximum change in ORI scores post-induction and the time when such change happened (peak time) during the 1-hour observing period. Representative images show the development of ocular redness, and the graph summarizes the kinetics of change in ORI score with data representing mean±SEM. Results show the 5% dapiprazole inducing the highest increase in ORI score. *, p<0.05, †, p<0.01 for 5% dapiprazole vs. PBS.

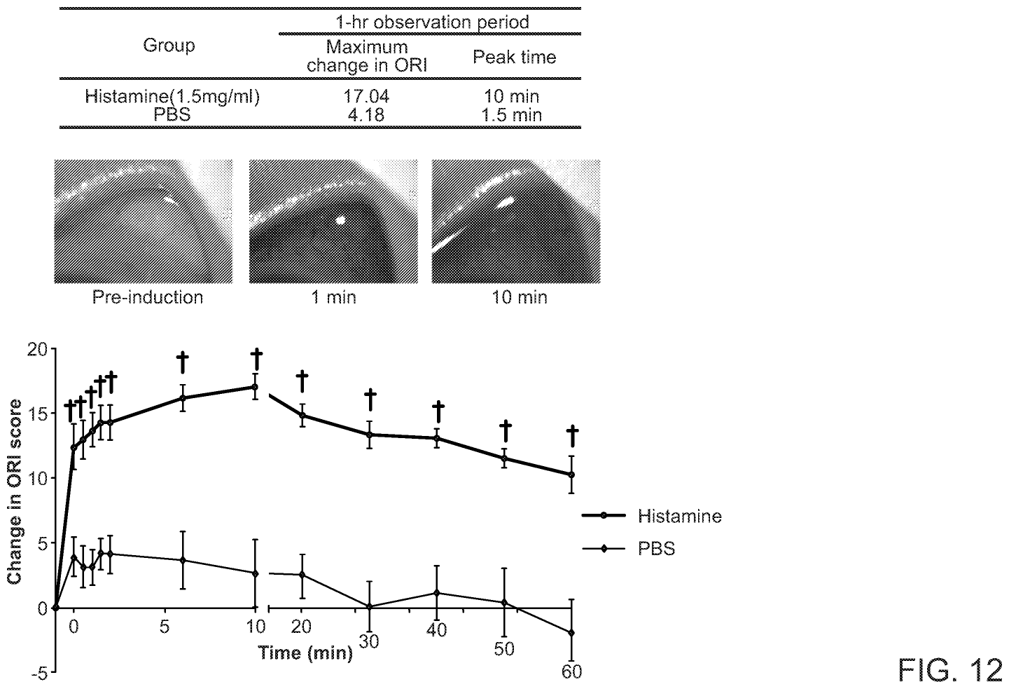

FIG. 12 shows the results obtained from a Guinea pig model of allergic ocular redness. Animals were anesthetized, and 20 μl of 1.5 mg/ml histamine was applied to the right eyes of animals to induce ocular redness. PBS was used as a control on the left eyes. Eyes were examined by a slit lamp, and images were taken pre-induction, and every 30 seconds for the first 2 minutes post-induction, followed by every 4 minutes for additional 8 minutes and every 10 minutes until 1 hour. The table summarizes the maximum change in ORI scores post-induction and the time when such change happened (peak time) during the 1-hour observing period. Representative images show the development of ocular redness, and the graph summarizes the kinetics of change in ORI score with data representing mean±SEM. †, p<0.01 as compared to PBS.

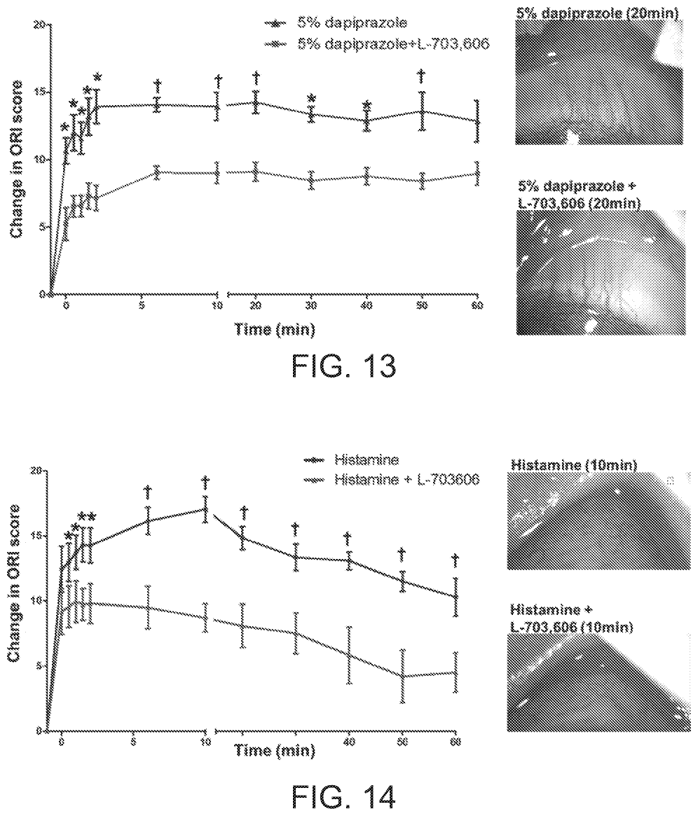

Neurokinin-1 receptor antagonism was shown to ameliorate non-allergic ocular redness (FIG. 13). Animals were topically treated with 1.0mg/m1 L-703,606 (a highly selective NK1R antagonist) at the same time of 5% dapiprazole instillation in 40 μl volume. Animals induced with dapiprazole but without L-703,606 treatment served as the control. Eyes were examined by a slit lamp, and images were taken pre-induction, and every 30 seconds for the first 2 minutes post-induction, followed by every 4 minutes for additional 8 minutes and every 10 minutes until 1 hour. The kinetics of change in ORI score with data representing mean±SEM is summarized. Results show blockade of SP/NK1R rapidly and consistently ameliorating the ocular redness. *, p<0.05, †, p<0.01 as compared to control.

Results shown in FIG. 14 demonstrated that neurokinin-1 receptor antagonism ameliorates allergic ocular redness. Animals were topically treated with 1.0 mg/ml L-703,606 (a highly selective NK1R antagonist) at the same time of 1.5 mg/ml histamine instillation in 20 μl volume. Animals induced with histamine but without L-703,606 treatment served as the control. Eyes were examined by a slit lamp, and images were taken pre-induction, and every 30 seconds for the first 2 minutes post-induction, followed by every 4 minutes for additional 8 minutes and every 10 minutes until 1 hour. The kinetics of change in ORI score with data representing mean±SEM is summarized. Results show blockade of SP/NK1R rapidly and consistently ameliorating the ocular redness. *, p<0.05, †, p<0.01 as compared to control.

One (1) drop of NK1R antagonist is applied to the animal eyes during the 1-hour study period. The optimal dosage and frequencies are evaluated according to the needs of the subject using methods known in the art or as determined by a physician. Treatments often require 4 times daily application.

Exemplary treatment regimens are described below. For clinical use in humans, a composition is administered to the eye of the subject at least 1, 2, 3, 4, 5, or 6 times per day, about 1, 2, 3, 4, 5, 6, or 7 times per week. A symptom of the ocular immunoinflammatory disorder is reduced within about 5, 15, 30, or 60 minutes; or within 1, 2, 3, 4, 5, 6, 7, 8, 9, 10, 11, 12, 13, or 14 days after administration of an inhibitor.

Typically 1 drop of 0.1˜10 μg/μL eye drops are delivered topically 1 to 4 times per day for a duration of as short as 1-2 days for ocular redness or as long as indefinite (life-long) in the case of dry eye therapy. In some cases, administration may be less than once daily.

### Example 5: Measurement of Treg-associated Biomarkers (TGFβ, IL-10) on Ocular Surface

To monitor the therapeutic efficacy of NK1R antagonist on Treg-associated ocular immunoinflammatory disorder, unstimulated tear is can be collected from the patients (Massingale, M. L., et al., 2009. Analysis of inflammatory cytokines in the tears of dry eye patients. Cornea 28, 1023-1027) is quantified for the protein levels of Treg functional biomarkers, such as TGF-β and IL-10, using ELISA. In addition, conjunctival samples are collected using the impression cytology technique (De Paiva, C. S., et al. 2009. IL-17 disrupts corneal barrier following desiccating stress. Mucosal. Immunol. 2, 243-253), and analyzed for mRNA levels of TGF-β and IL-10.

### Example 6: Neurokinin-1 Receptor Antagonism Ameliorates Dry Eye Disease by Inhibiting Antigen-Presenting Cell Maturation and T_{H}17 Cell Activation

DED-induced alterations in SP expression were evaluated and the effect of SP derived from stimulated corneal nerve endings on APC maturation was investigated, a key step in activation of effector T_{H}17 mechanisms in DED. Furthermore, the efficacy of blocking SP signaling using NK1R antagonists in reducing DED severity was evaluated. The results described herein show that SP is constitutively expressed at the ocular surface, and its expression is upregulated in the course of DED. Using in vitro studies, it was demonstrated that SP augments the maturation of bone marrow-derived dendritic cells and that antagonizing NK1R abrogates this effect. Finally, using a well-established mouse model of DED, it was shown that treatment of DED mice with topical NK1R antagonists CP-99,994 and L-733,060 suppresses APC maturation and T_{H}17 cell activation, and significantly reduces disease severity.

The following materials and methods were used in the studies described herein.

Animals: Eight to nine-week old female C57BL/6 mice (Charles River Laboratories, Wilmington, Mass.) were used in these experiments.

Induction of Dry Eye Disease (DED): DED was induced by housing the mice in a low-humidity (relative humidity: <20%) controlled environment chamber (CEC) with constant airflow of 15 L/min and temperature of 21 to 23° C. for 14 days. Age- and sex-matched mice housed in room air conditions served as controls. Corneal epithelial disease was evaluated using fluorescein (Sigma-Aldrich) staining and scored using the National Eye Institute grading system (NEI, Bethesda, Md.; Chen Y. et al. IFN-gamma-Expressing T_{H}17 Cells Are Required for Development of Severe Ocular Surface Autoimmunity. J Immunol 2017, 199:1163-9). 1 μl of 2.5% fluorescein was applied into the lateral conjunctival sac of the mice and after 3 minutes corneas were examined with a slit lamp biomicroscope under cobalt blue light. Punctate staining was recorded in a masked fashion with the standard National Eye Institute grading system of 0-3 for each of the five areas of the cornea.

Topical Treatment with NK1R Antagonist: Mice were assigned to one of four groups (n=5 each). 1 μg/μl of NK1R antagonists CP-99,994, L-733,060 (R&D systems, Minneapolis, MN) or PBS was administered topically three times per day from day 4 to day 14 after DED induction. Untreated DED mice served as controls.

Generation of Bone Marrow-Derived Dendritic Cells (BMDCs): Long bones (femur and tibia) were harvested from C57BL/6 mice and cell suspension was prepared. Cells were incubated with Red blood cell (RBC) lysis buffer (Sigma-Aldrich, St. Louis, Mo.) at 37° C. for 10 minutes. Bone marrow cells were plated at the concentration of 5×10⁶ cells in 5 mL RPMI-1640 medium/well (Lonza Biologics, Inc., Hopkinton, Mass., USA) supplemented with 5% heat inactivated fetal calf serum (Atlanta Biologicals, Flower Branch, Ga.), 2 mM L-glutamine (Lonza Biologics, Inc.), 100 U/mL of penicillin (Lonza Biologics, Inc.), 100 μg/mL of streptomycin (Lonza Biologics, Inc.), 50 mM 2-mercaptoethanol (Sigma-Aldrich), and 20 ng/mL of granulocyte/macrophage colony-stimulating factor (GM-CSF, Biolegend, San Diego, Calif.) for 6 to 7 days. Lymphocytes were removed by washing on days 2 and 4 of culture. On day 7, non-adherent and loosely adherent immature BMDCs were harvested. To activate BMDCs, immature BMDCs were cultured in the presence of 20 ng/mL of IL-1β (PeproTech, Rocky Hill, N.J.) in 6-well plates for 24 hours.

Primary Trigeminal Ganglion Culture: Trigeminal ganglions (TGs) were cultured using known methods, e.g., Bertke A S, et al. A5-positive primary sensory neurons are nonpermissive for productive infection with herpes simplex virus 1 in vitro. J Virol 2011, 85:6669-77. TGs were harvested from 6 to 8-week-old C57BL/6 mice, and were digested with papain followed by collagenase type II/dispase (Invitrogen). The TGs were selected by the lower layers of the 5-layered OptiPrep density gradients (Sigma-Aldrich). Neurons were counted and plated on Poly-L-Lysine and Laminin- coated 4-chamber slides or 24-well plates at a density of 3,000 cells per well (Malin S A, et al. Nat Protoc 2007, 2:152-60). Neuronal cultures were maintained with complete neuronal medium: Neurobasal A medium (Invitrogen, Cat. No. 10888-022) supplemented with 2% B27 supplement; 1% penicillin & streptomycin; L-glutamine (500 μM); nerve growth factor (NGF; 50 ng/ml); glial-cell-derived neurotrophic factor (GDNF; 50 ng/ml), and the mitotic inhibitors fluorodeoxyuridine (40 μM) and aphidicolin (16.6 μg/ml) for the first 3 days. The medium was then replaced with fresh medium without fluorodeoxyuridine and aphidicolin (growth factors were from R&D Systems, and other supplements were from Sigma).

Bone Marrow-Derived Dendritic Cell and Trigeminal Ganglion Co-culture: Primary TG culture was performed, and loosely adherent immature BMDCs were collected after 7 days of culture. After counting the number of BMDCs, the cell density was adjusted to 150,000 cells/mL in BMDC culture media (based on RPMI-1640) without GM-CSF. After removal of the TG culture medium, 1 mL of media containing immature BMDCs was added to each well. After 2 hours, IL-1β (20 ng/10 μL) was added to each well to induce BMDC maturation, and cells were co-cultured for 18 hours.

Single Cell Suspension Preparation and Flow Cytometry: Submandibular and cervical draining lymph nodes (DLNs) were harvested and single cell suspensions were prepared. Cells were stimulated with phorbol 12-myristate 13-acetate (PMA, 50 ng/mL; Sigma-Aldrich) and Ionomycin (500 ng/mL; Sigma-Aldrich) for 6 hours in the presence of a commercial protein transport inhibitor (0.7 μL/100 μL media, Golgistop; BD Biosciences, San Jose, Calif.). Conjunctivae were harvested by lifting at the junction of bulbar and palpebral conjunctiva and dissecting along both bulbar and palpebral insertion points (Vannas Scissors; Storz, Bausch & Lomb). Conjunctiva samples were cultured in RPMI (Thermo Fisher Scientific, Waltham, Mass.) supplemented with 10% fetal bovine serum (FBS) and stimulated with PMA (50 ng/mL; Sigma-Aldrich) and Ionomycin (500 ng/mL; Sigma-Aldrich) in the presence of a protein transport inhibitor (0.7 μL/100 μL media, Golgistop; BD Biosciences) at 37° C. for 24 hours. Harvested corneas were digested in RPMI media containing 2 mg/ml collagenase type IV (Sigma-Aldrich) and 2 mg/ml DNase I (Roche) for 1 h at 37° C. The suspension was then filtered through a 70-μm cell strainer. Single-cell suspensions of DLN and cornea were stained with the following antibodies: Brilliant Violet 421-conjugated anti-mouse I-A/I-E (MHC-II), PE-conjugated anti-CD11c (BD Pharmingen, San Jose, Calif.), PerCP/Cy5.5-conjugated anti-CD11b, FITC-conjugated anti-CD4 (BioLegend, San Diego, Calif.), and PE-conjugated anti-interleukin 17A (eBioscience, San Diego, Calif.). Intranuclear staining with PE/Cy7-conjugated anti-Foxp3 (eBioscience) was performed after fixation and permeabilization of cells. Control samples were stained with appropriate isotype-matched antibodies. Stained cells were examined using an LSRII Flow Cytometer (BD Biosciences, Franklin Lakes, N.J., USA), and data were analyzed using commercial Summit software (Summit v4.3; Dako Colorado, Inc., Fort Collins, Colo.).

Enzyme-Linked Immunosorbent Assay: For protein extraction, cornea and trigeminal ganglion were harvested and stored in cold sterile PBS containing protease inhibitors (Sigma-Aldrich) at −80° C. until used. The samples were homogenized on ice and centrifuged. The supernatant was assayed for levels of SP using commercial competitive enzyme immunoassay kit (R&D Systems).

Real-Time PCR: Bulbar and palpebral conjunctiva, cornea and trigeminal ganglion were harvested from mice and stored in TRIzol reagent (Invitrogen, Carlsbad, Calif.) at −80° C. until RNA was isolated and reverse-transcribed using RNeasy micro kit (Qiagen, Valencia, Calif.) and SuperScript III kit (Invitrogen). Real-time PCR was performed using TaqMan Universal PCR Master Mix and predesigned primers for IL-17A (Mm00439618_ml), SP (Mm01166996_ml), and glyceraldehyde 3-phosphate dehydrogenase (GAPDH, Mm99999915_gl) (Applied Biosystems, Carlsbad, Calif.). Samples were analyzed using a real-time PCR system (LightCycler 480 II System; Roche Applied Science, Indianapolis, Ind.), and results were analyzed by the comparative threshold cycle method, using GAPDH as an internal control.

Statistical Analysis: Unpaired student's t test was performed for comparison between two groups, and p<0.05 was considered statistically significant. Results are presented as mean±SEM of at least 2 independent experiments.

### Substance P Expression Increases in the Course of Dry Eye Disease

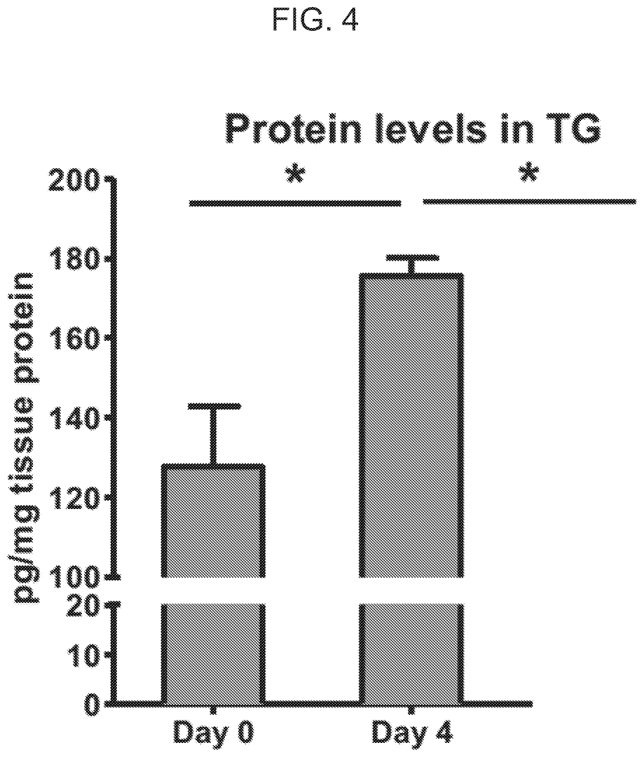

To evaluate expression levels of SP in different phases of DED, SP mRNA and protein levels were measured in the cornea and trigeminal ganglion (TG) of normal and DED mice. A well-known mouse model of DED was adopted, in which DED was induced by housing the animals in a controlled environment chamber for 2 weeks, as previously shown (Chauhan S K, et al., A novel pro-lymphangiogenic function for T_{H}17/IL- 17. Blood 2011, 118:4630-4). Corneas and TGs were harvested on days 4 and 14 after DED induction, and protein and mRNA levels of SP in both tissues were evaluated using ELISA and real-time PCR, respectively. SP protein levels were assessed in the TG culture supernatant using ELISA. Our results demonstrated baseline expression of SP in both the cornea and TG, levels of which were upregulated in both tissues after induction of DED (FIG. 4, p=0.01 and p=0.003, respectively). By day 14, SP protein levels in both the cornea and TG returned to near normal values. At the mRNA level, expression of SP in corneas of DED mice was significantly higher than healthy controls (0.84 fold on day 4, p=0.001; 1.43 fold on day 14, p=0.02). Furthermore, in the TG, SP mRNA levels were significantly higher than those seen in the cornea, and increased expression induced by desiccating stress occurred later in the course of DED. These results show constitutive expression of SP at the ocular surface mainly in the TG neurons, levels of which are upregulated in response to desiccating stress.

SP Derived from Trigeminal Nerves Promotes Bone Marrow-Derived Dendritic Cell Maturation In Vitro

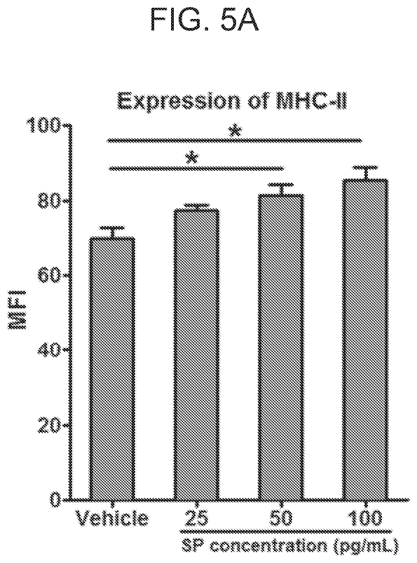

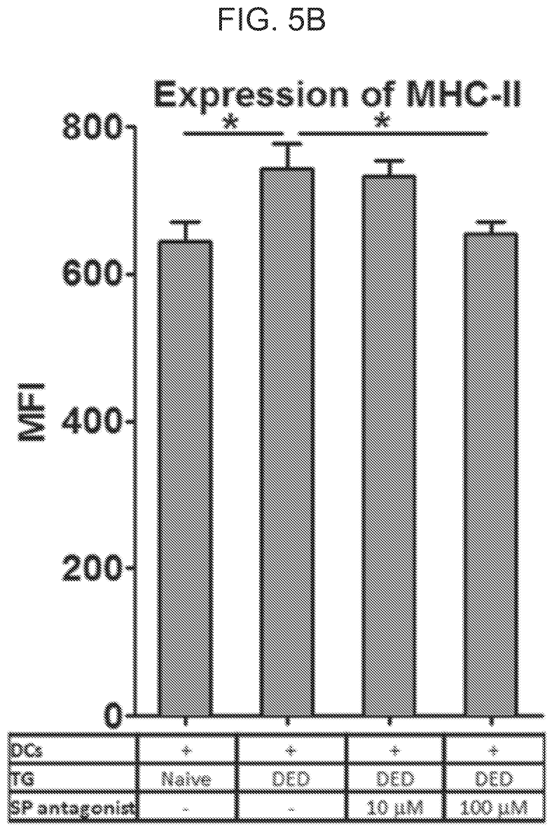

Mature APCs play a key role in the activation of T_{H}h17 cells and the autoimmune response in DED (Barabino S. et al. Prog Retin Eye Res 2012, 31:271-85; Stevenson W, Chauhan S K, Dana R: Dry eye disease: an immune-mediated ocular surface disorder. Arch Ophthalmol 2012, 130:90-100; Hamrah P, et al. Invest Ophthalmol Vis Sci 2003, 44:581-9). Given that increased SP levels in response to desiccating stress was observed, the effect of SP on maturation of APCs was next evaluated. Immature bone marrow-derived dendritic cells (BMDCs) were cultivated by culturing murine bone marrow cells with 20 ng/mL GM-CSF for 6 days. BMDCs were subsequently primed with 20 ng/mL IL-1β in the absence of GM-CSF for 24 hours. Primed BMDCs were cultured in the presence of three different concentrations of SP (25, 50 and 100 μg/mL) for 18 hours, and expression of MHC II maturation marker by BMDC was evaluated using flow cytometry (FIG. 5A). The results demonstrated that increasing concentrations of SP led to a significant and dose-dependent increase in the mean fluorescence intensity (MFI) of MHC II in CD11 BMDCs (p=0.03 and p=0.02 for SP 50 and 100 μg/mL vs. vehicle, respectively). To investigate the role of TG neurons (which innervate the cornea) on BMDC maturation, TGs harvested from DED or control mice were cultured for 3 days and subsequently co-cultured with primed BMDCs for 18 hours. In addition, two different doses of neurokinin-1 receptor antagonist, Spantide (10 and 100 μM), were added to the co-culture system to block the SP signaling. The expression of MHC II maturation marker by CD11c⁺ BMDCs was analyzed in different groups using flow cytometry. As shown in FIG. 5B, TG derived from DED mice induced a significant increase in expression of MHC II by BMDC compared to TG derived from normal healthy mice (743±32 vs. 644±26; p=0.04). Finally, blockade of SP with Spantide at the concentration of 100 μM, significantly abrogated the effect of DED TG on BMDC maturation, (p=0.04, FIG. 5B), implicating SP derived from TG neurons in the upregulated expression of MHC II by BMDCs.

### NK1R Antagonists Ameliorate DED and Inhibit Antigen-Presenting Cell Maturation in the Cornea and Draining Lymph Nodes

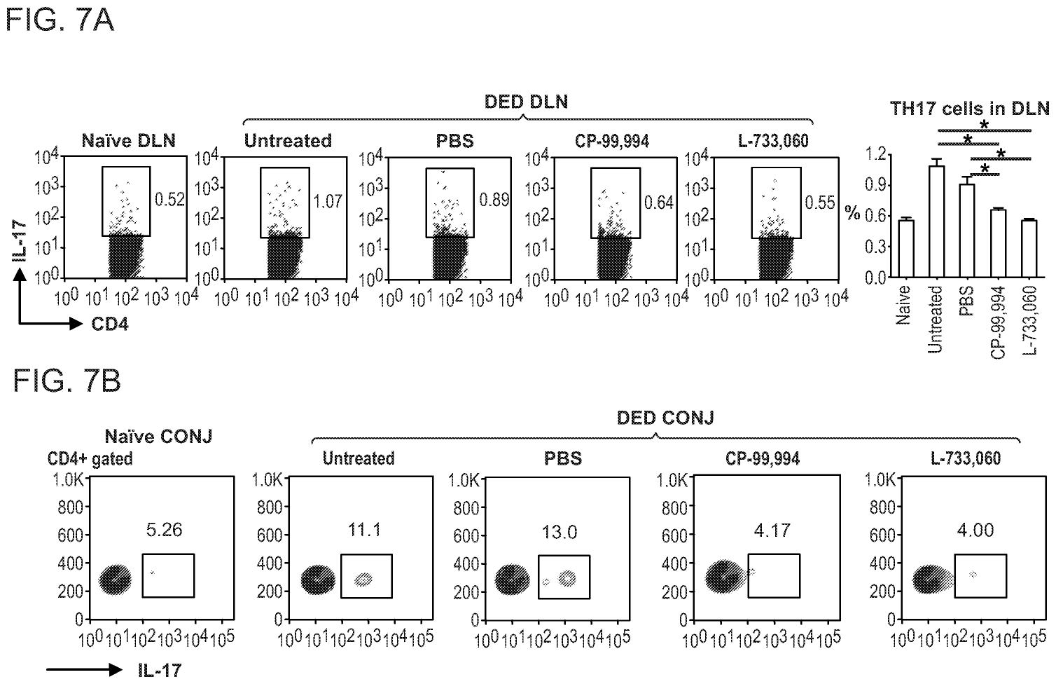

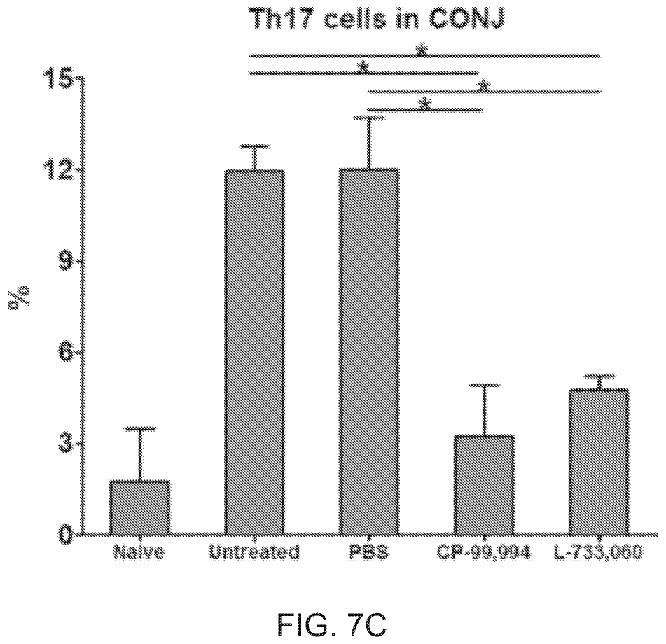

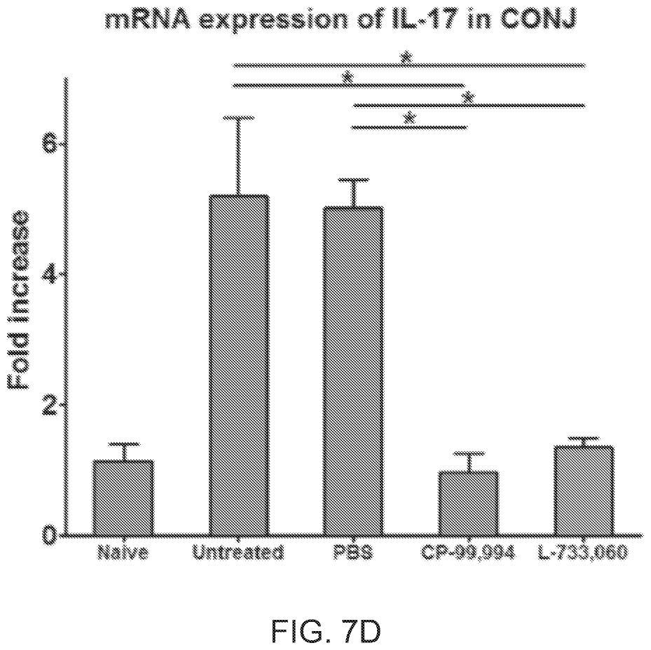

Having shown the in vitro effect of NK1R antagonists in abrogating SP-induced APC maturation, the effect of topical blockade of SP receptor on the clinical signs of DED was examined. Following the induction of DED for 4 days, mice received topical application of either NK1R antagonist CP-99,994, L-733,060 or PBS control three times per day until day 14 after DED induction. As shown in FIG. 6A, compared with untreated and PBS-treated controls, topical application of either NK1R antagonists CP-99,994 or L-733,060 significantly reduced CFS scores and DED severity at day 7, 10 and 14 after DED induction (p<0.05). To confirm the effect of topical blockade of SP on APC maturation in DED mice, we examined the frequencies of MHC-IIhi CD11b+cells in the cornea and DLNs using flow cytometry (FIGS. 7B and 7C). The cornea of untreated and PBS-treated DED mice showed a significant increase in the frequencies of MHC-II^{hi} CD11b⁺cells compared with that of naive mice. However, the frequencies of MHC-II^{hi} CD11b⁺ cells in the cornea were significantly lower in both CP-99,994 and L-733,060-treated mice (FIG. 6B). Similarly, the DLNs of CP-99,994 and L-733,060-treated mice showed a significant decrease in the frequencies of mature MHC-II^{hi} CD11b⁺ cells (FIGS. 7C and 7D; p=0.005 for CP-99,994 vs. PBS; p=0.001 for L-733,060 vs. PBS) as well as the level of MHC-II expression by CD11b⁺ cells (FIG. 6E, p=0.003 for CP-99,994 vs. PBS; p=0.02 for L-733,060 vs. PBS) compared with that of PBS-treated mice.

NK1R Antagonists Suppress Activation of T_{H}17 Cells in DLNs and their Infiltration in the Conjunctivae

The effect of topical blockade of SP signaling on T_{H}17 cell activation in DLNs and conjunctivae was examined next. The DLNs of untreated and PBS-treated mice showed an increase (1- to 1.5-fold) in the frequencies of T_{H}17 cells compared with that of naive mice (p=0.003 and p=0.013, respectively). However, topical application of either NK1R antagonist significantly decreased the frequencies of T_{H}17 cells in the DLNs (FIGS. 8A and 8B, p=0.02 for CP-99,994 vs. PBS, and p=0.01 for L-733,060 vs. PBS). A similar decreasing trend was observed in the frequencies of T_{H}17 cells in the conjunctivae of NK1R antagonist-treated mice (FIGS. 8C and 8D, p=0.02 for CP-99,994 vs. PBS, and p=0.01 for L-733,060 vs. PBS). In addition, the real-time PCR data demonstrated a significant reduction in mRNA expression levels of inflammatory cytokine IL-17 in conjunctivae of NK1R antagonist-treated mice (p=0.001 for CP-99,994 vs. PBS and L-733,060 vs. PBS), further confirming the effect of blocking SP signaling on suppressing the T_{H}17 immune response in DED mice.

### NK1R Antagonism Reduces Severity and Symptoms of DED

Neurogenic inflammation has been implicated as a potential mechanism involved in the development and chronicity of DED (Beuerman R W, et al., Ocul Surf2005, 3:S203-6; Stern M E, et al., The role of the lacrimal functional unit in the pathophysiology of dry eye. Exp Eye Res 2004, 78:409-16). However, the precise role of neuromodulators such as SP in the pathogenesis of DED heretofore has not been elucidated (Sabatino F. et al. The Intriguing Role of Neuropeptides at the Ocular Surface. Ocul Surf 2017, 15:2-14). Herein, it was demonstrated that TG is the major source of SP in the course of DED. It was shown herein that SP derived from TG enhances the expression of MHC II by BMDCs (a critical mechanism linked to DC antigen presenting function), and that this effect is abrogated by blockade of SP signaling using NK1R antagonist Spantide. Finally, using a well-established murine model of DED it was also shown that topical treatment of DED mice with NK1R antagonists CP-99,994 and L-733,060 suppresses APC acquisition of MHC II, reduces T_{H}h17 cell infiltration and activity, and ameliorates DED severity.

The cornea is the most richly innervated tissue in the body, which receives dense sensory nerve fibers from the ophthalmic branch of the trigeminal nerve. A dense network of SP-expressing fibers innervates the basal area of the corneal epithelium, with terminal branches that penetrate the more superficial layers. SP is known as a key molecule in the cross-communications between neural and immune systems. Although neuronal cells serve as the main source of SP, endogenous expression of SP has also been demonstrated in immune cells, keratocytes and epithelial cells (O'Connor T. M. et al., J Cell Physiol 2004, 201:167-80; Watanabe M. et al. Jpn J Ophthalmol 2002, 46:616-20). Thus far, few studies have studied the alterations in SP levels in DED. The results described herein demonstrated significantly higher baseline expression levels of SP in the TG neurons compared to the cornea, both of which are upregulated in response to desiccating stress. The fact that neuronal SP mRNA levels in these experiments increase at a later time point after induction of DED provide evidence that the early expression of SP at the protein level could likely be due to the pre-formed protein in the corneal epithelium.

Antigen presenting cells (APCs), including dendritic cells (DCs) play a pivotal role in the regulation of immune responses at the interface of innate and adaptive immunity (Fransen J. H. et al. Arthritis Res Ther 2010, 12:207). A heterogeneous population of tissue resident DCs has been described in the corneal epithelium and stroma (Hamrah P, Huq S O, Liu Y, Zhang Q, Dana M R. J Leukoc Biol 2003, 74:172-8). Under inflammatory conditions, these DCs undergo the maturation process and acquire antigen presenting capacity to stimulate T lymphocyte-dependent responses (Liu Y. J. et al. Nat Immunol 2001, 2:585-9). The presence of mature MHC DCs has been observed in the course of a wide variety of immunoinflammatory conditions, including dry eye disease (Catry L, et al. Graefes Arch Clin Exp Ophthalmol 1991, 229:182-5). The homing of mature APCs from the ocular surface to the DLN is a critical step in the early immunopathogenesis of DED (Barabino S, Chen Y, Chauhan S, Dana R. Prog Retin Eye Res 2012, 31:271-85; Stevenson W, Chauhan S K, Dana R. Arch Ophthalmol 2012, 130:90-100). APC-mediated priming of effector T cells has been proposed as a potential source of autoimmunity in DED (Barabino S, Chen Y, Chauhan S, Dana R: Ocular surface immunity: Homeostatic mechanisms and their disruption in dry eye disease. Progress in Retinal and Eye Research 2012, 31:271-85). The results of the TG-BMDC co-culture demonstrate that TG-derived SP enhances the expression of MHC class II by DCs, thus enhancing their antigen presenting capacity. Antagonizing NK1R in the co-culture interestingly abrogates SP-induced DC maturation, further demonstrating the role of SP signaling in inducing APC activation.

Heretofore, the efficacy of NK1R antagonists in animal models of DED has not as of yet been described. To evaluate their efficacy in the setting of DED, two different, highly specific and potent NK1R antagonists were tested: CP-99,994, and L-733,060. The results showed that topical application of either CP-99,994 [(2S,3S)—N-[(2-Methoxyphenyl)methyl]-2-phenyl-3-piperidinamine dihydrochioride] or L-733,060 [(2S,3S)-3-[[3,5-bis(Trifluoromethyl)phenyl]methoxy]-2-phenylpiperidine hydrochloride] suppressed APC maturation and T_{H}17 activity, resulting in amelioration of corneal epitheliopathy. The application of CP-99,994 has not been previously reported in ocular diseases. However, SP is a pleiotropic molecule with myriad functions, including in physiologic homeostasis of the ocular surface. Topical application of SP promotes corneal epithelial wound healing in diabetic mice and subconjunctival injection of L-733,060 significantly inhibits the protective role of SP on epithelial healing (Yang L, et al. Diabetes 2014, 63:4262-74).

In summary, the data herein, on the increased frequencies of MHC-II^{hi} CD11b⁺ APCs in the cornea and DLNs after the induction of DED demonstrated the effect of desiccating stress in enhancing the maturation of resident corneal APCs and their migration toward the DLNs. It was also shown that treatment of DED mice with NK1R antagonists significantly decreased the frequencies of mature APCs in the cornea and DLNs, where APCs prime naïve T cells to differentiate into IL-17-secreting T_{H}17 cells. The observed suppressive effect of NK1R antagonists on APC maturation prompted the assessment of their efficacy in inhibiting T_{H}17 cell activation. A substantial decrease in the frequencies of T_{H}17 cells was observed both in the DLNs and conjunctivae of NK1R antagonist-treated mice, which was associated with a marked reduction in IL-17 mRNA levels.

The findings described herein provide evidence that antagonizing NK1R-mediated signaling is effective in suppressing T_{H}17-mediated ocular surface disease and reducing the severity of DED.

### Example 7: Restoration of Regulatory T cell Function in Dry Eye Disease by Targeting Substance P/Neurokinin 1 Receptor

The purpose of these experiments were to assess the phenotypic and functional changes in Tregs in response to SP, and to evaluate the role of blocking neurokinin 1 receptor (NK-1R) in restoring Treg function in a mouse model of DED.

CD4⁺CD25⁺Foxp3⁺ Tregs were isolated from draining lymph nodes (DLN) of naïve female C57BL/6 mice.

Isolated Tregs were co-cultured with SP (1 μM) with or without Spantide I (10 μM) for 48 hours. Phenotype of Tregs was evaluated by flow cytometry, and the capacity of Tregs to suppress effector T cell proliferation was investigated.

DED was induced in mice for 14 days. Spantide I or PBS (control) was administered intraperitoneally (36 μg/day) from one day before DED induction until day 14. Suppressive function of Tregs in DLN, frequencies of T_{H}17 cells in DLN and conjunctivae, and severity of corneal epitheliopathy were evaluated.

The results demonstrate that substance P induces Treg dysfunction by suppressing Treg expression of inhibitory molecules and secreted immunomodulatory cytokines, which is reversed by Spantide I (FIGS. 9A-9C). Inhibition of SP signaling through systemic administration of Spantide I restores Treg function, suppresses T_{H}17 cells and reduces the severity of DED (FIGS. 10A-10D).

SP induces Treg dysfunction by suppressing Treg expression of inhibitory molecules and immunomodulatory cytokines. SP-induced Treg dysfunction is reversed by the NK-1R antagonist, Spantide I. Treatment of DED mice with systemic Spantide I restores the suppressive function of Tregs, suppresses T_{H}17 cells, and ameliorates DED severity.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of treating a non-infectious ocular immunoinflammatory disorder in a subject comprising administering to said subject a composition comprising one or more neurokinin 1 receptor (NK1R) antagonists, wherein said subject is diagnosed with or suffering from a regulatory T cell (Treg)-associated ocular disorder.

2. The method of claim 1, wherein said composition comprises L-733,060 or L-703,060.

3. The method of claim 1, wherein said Treg-associated ocular disorder is one selected from non- Dry Eye Disease (DED)-related ocular redness, Dry Eye Disease (DED), allergic conjunctivitis and/or ocular pain, and said non-DED-related ocular redness comprises allergic ocular redness or non-allergic ocular redness.

4. (canceled)

5. The method of claim 1, wherein said NK1R antagonist is one selected from a small molecule antagonist of NK1R, a neutralizing anti-NK1R antibody, a blocking fusion protein against SP, an anti-SP antibody or a nucleic acid.

6. (canceled)

7. The method of claim 1, wherein said NK1R antagonist comprises: Spantide (RPKPQQWFWLL; SEQ ID NO: 2), 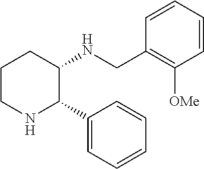 [SMILES: COc1ccccc1CN[C@H]1CCCN[C@H]1c1ccccc1]
(2S,3S)—N-[(2-Methoxyphenyl)methyl]-2-phenyl-3-piperidinamine dihydrochloride, 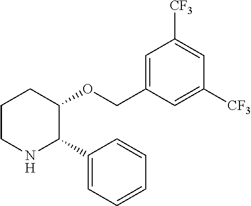 [SMILES: FC(F)(F)c1cc(CO[C@H]2CCCN[C@H]2c2ccccc2)cc(C(F)(F)F)c1]
(2S,3S)-3-[[3,5-bis(Trifluoromethyl)phenyl]methoxy]-2-phenylpiperidine hydrochloride, 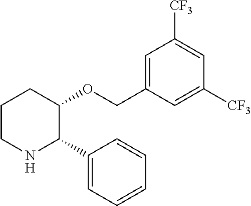 [SMILES: FC(F)(F)c1cc(CO[C@H]2CCCN[C@H]2c2ccccc2)cc(C(F)(F)F)c1]
(2S,3S)-3-[[3,5-bis(Trifluoromethyl)phenyl]methoxy]-2-phenylpiperidine hydrochloride, 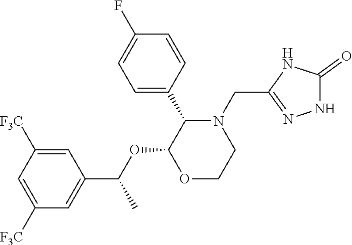 [SMILES: C[C@@H](O[C@H]1OCCN(Cc2n[nH]c(=O)[nH]2)[C@H]1c1ccc(F)cc1)c1cc(C(F)(F)F)cc(C(F)(F)F)c1]
5-[[(2R,3S)-2-[(1R)-1-[3-Bis(trifluoromethyl)phenyl]ethoxy]-3-(4-fluorophenyl)-4-morpholinyl]methyl]-1,2-dihydro-3H-1,2,4-triazol-3-one, 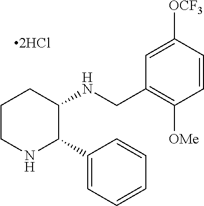 [SMILES: COc1ccc(OC(F)(F)F)cc1CN[C@H]1CCCN[C@H]1c1ccccc1]
(2S,3S)—N-[[2-Methoxy-5-(trifluoromethoxy)phenyl]methyl]-2-phenyl-3-piperidinamine dihydrochloride, 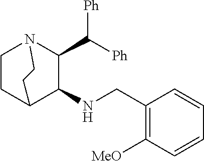 [SMILES: COc1ccccc1CN[C@H]1C2CCN(CC2)[C@H]1C(c1ccccc1)c1ccccc1]
(2S,3S)—N-(2-Methoxyphenyl)methyl-2-diphenylmethyl-1-azabicyclo[2.2.2]octan-3-amine, 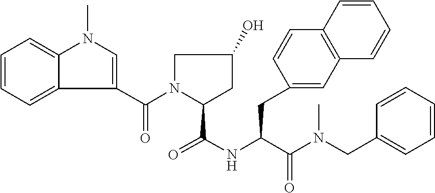 [SMILES: CN(Cc1ccccc1)C(=O)[C@H](Cc1ccc2ccccc2c1)NC(=O)[C@@H]1C[C@@H](O)CN1C(=O)c1cn(C)c2ccccc12]
(4R)-4-Hydroxy-1-[(1-methyl-1H-3-yl)carbonyl]-L-prolyl-N-methyl-3-(2-naphthalenyl)-N-(phenylmethyl)-L-alaninamide, 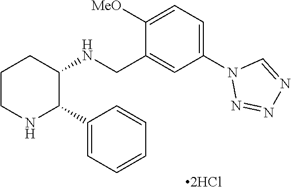 [SMILES: COc1ccc(-n2cnnn2)cc1CN[C@H]1CCCN[C@H]1c1ccccc1]
(2S,3S)—N-[[2-Methoxy-5-(1H-tetrazol-1-yl)phenyl]methyl]-3-piperidinamine dihydrochiloride, 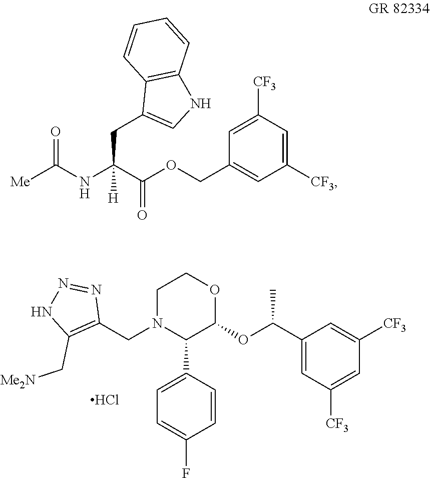
5-[[(2R,3S)-2-[(1R)-1-[3,5-Bis(trifluoromethyl)phenyl]ethoxyl]-3-(4-fluorophenyl)-4-morpholinyl]methyl-N,N-dimethyl-1H-1,2,3-triazole-4-methanamine hydrochloride, 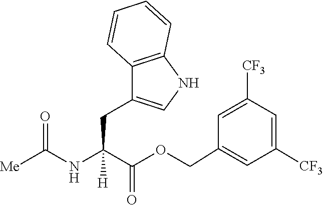
N-Acetyl-L-tryptophan 3,5-bis(trifluoromethyl)benzyl ester, 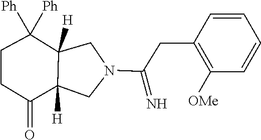 [SMILES: COc1ccccc1CC(=N)N1C[C@@H]2C(=O)CCC(c3ccccc3)(c3ccccc3)[C@@H]2C1]
(3aR,7aR)-Octahydro-2-[1-imino-2-(2-methoxyphenyl)ethyl]-7,7-diphenyl-4H-isoindol, 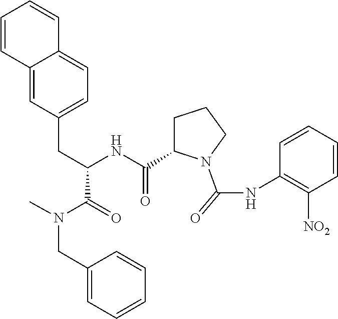 [SMILES: CN(Cc1ccccc1)C(=O)[C@H](Cc1ccc2ccccc2c1)NC(=O)[C@@H]1CCCN1C(=O)Nc1ccccc1[N+](=O)[O-]]
1-[[(2-Nitrophenyl)amino]carbonyl]-L-prolyl-N-rnethyl-3-(2-naphthalenyl)-N-(phenylmethyl)-L-alaninamide; 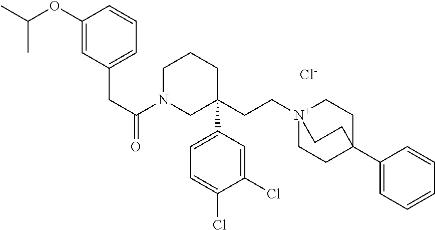 [SMILES: CC(C)Oc1cccc(CC(=O)N2CCC[C@](CC[N+]34CCC(c5ccccc5)(CC3)CC4)(c3ccc(Cl)c(Cl)c3)C2)c1 ; [Cl-]]
1-[2-[(3S)-3-(3,4-Dichlorophenyl)-1-[2-[3-(1-methylethoxy)phenyl]acetyl]-3-piperidinyl]ethyl]-4-phenyl-1-azoniabicyclo[2,2,2]octane chloride, analogs, or combinations thereof; or
said nucleic acid is one selected from an aptamer, a small interfering RNA, a microRNA, a small hairpin RNA and an antisense nucleic acid.

8. (canceled)

9. The method of claim 1, wherein the composition is administered to said subject by a topical administration, a subconjunctival administration, intravitreal administration, subcutaneous administration, ocularly administration, or combinations thereof.

10. The method of claim 9, wherein the composition is topically administered to said subject at least once a day, twice per day, or 3 times per day.

11. (canceled)

12. (canceled)

13. (canceled)

14. The method of claim 1, wherein said composition is administered to said subject in combination with a secondary therapy or a secondary agent.

15. A method of reducing a symptom of a non-infectious ocular immunoinflammatory disorder in a subject, comprising; administering to said subject with a Treg-associated ocular disorder a composition comprising a therapeutically effective amount of an SP signaling blockade-inducing agent.

16. The method of claim 15, wherein said Treg-associated ocular disorder is one selected from non-DED-related ocular redness, Dry Eye Disease (DED), allergic conjunctivitis and ocular pain, and said SP signaling blockade-inducing agent is selected from an SP blocker, an SP antagonist, an SP receptor blocker and an SP receptor antagonist.

17. (canceled)

18. The method of claim 16, wherein said SP receptor is NK1R (SEQ ID NO:1).

19. The method of claim 16, wherein the composition is administered to said subject by a topical administration, a subconjunctival administration, an intravitreal administration, subcutaneous administration or combinations thereof, and the subcutaneous administration is administered to an eyelid, forehead or the combination thereof.

20. (canceled)

21. (canceled)

22. (canceled)

23. A method of treating keratoneuralgia, corneal hyperalgesia, corneal alodynia in a subject, comprising: administering to said subject a composition comprising a therapeutically effective amount of one or more neurokinin 1 receptor (NK1R) antagonists.

24. The method of claim 23, wherein said NK1R antagonist is one selected from a small molecule antagonist of NK1R, a neutralizing anti-NK1R antibody, a blocking fusion protein against SP, an anti-SP antibody or a nucleic acid.

25. (canceled)

26. The method of claim 24, wherein said NK1R antagonist comprises: Spantide (RPKPQQWFWLL; SEQ ID NO: 2) or variants thereof, 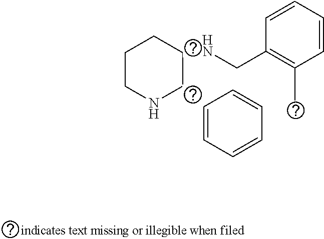
(2S,3S)—N-[(2-Methoxyphenyl)methyl]-2-phenyl-3-piperidinamine dihydrochloride, 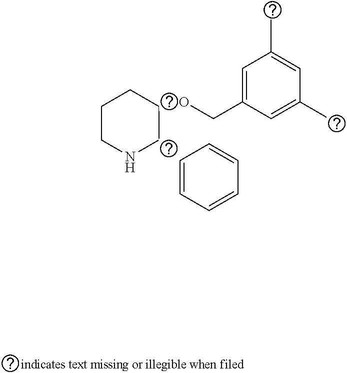
(2S,3S)-3-[[3,5-bis(Trifluoromethyl)phenyl]methoxy]-2-phenylpiperidine hydrochloride, 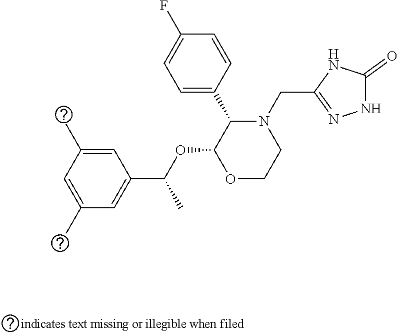 [SMILES: Cc1cc(C)cc([C@@H](C)O[C@H]2OCCN(Cc3n[nH]c(=O)[nH]3)[C@H]2c2ccc(F)cc2)c1]
5-[[(2R,3S)-2-[(1R)-1-[3,5-Bis(trifluoromethyl)phenyl]ethoxy]-3-(4-fluorophenyl)-4-morpholinyl]methyl]-1,2-dihydro-3H-triazol-3-one, 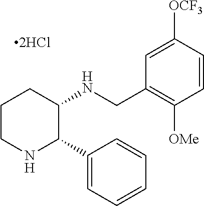 [SMILES: COc1ccc(OC(F)(F)F)cc1CN[C@H]1CCCN[C@H]1c1ccccc1]
(2S,3S)—N-[[2-Methoxy-5-(trifiuoromethoxy)phenyl]methyl]-2-phenyl-3-piperidinamine dihydrochloride, 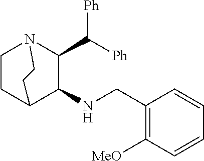 [SMILES: COc1ccccc1CN[C@H]1C2CCN(CC2)[C@H]1C(c1ccccc1)c1ccccc1]
(2S,3S)—N-(2-Methoxyphenyl)methyl-2-diphenylmethyl-1-azabicyclo[2.2.2]octan-3-amine, 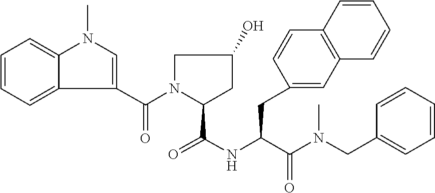 [SMILES: CN(Cc1ccccc1)C(=O)[C@H](Cc1ccc2ccccc2c1)NC(=O)[C@@H]1C[C@@H](O)CN1C(=O)c1cn(C)c2ccccc12]
(4R)-4-Hydroxy-[(1-methyl-1H-indol-3-yl)carbonyl]-L-prolyl-N-methyl-3-(2-naphthalenyl)-N-(phenylmethyl)-L-alaninamide, 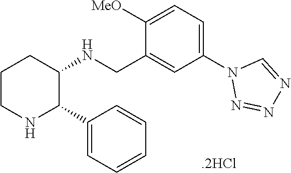 [SMILES: COc1ccc(-n2cnnn2)cc1CN[C@H]1CCCN[C@H]1c1ccccc1]
(2S,3S)—N-[[2-Methoxy-5-(1H-tetrazol-1-yl)phenyl]methyl]-2-phenyl-3-piperidinamine dihydrochloride, 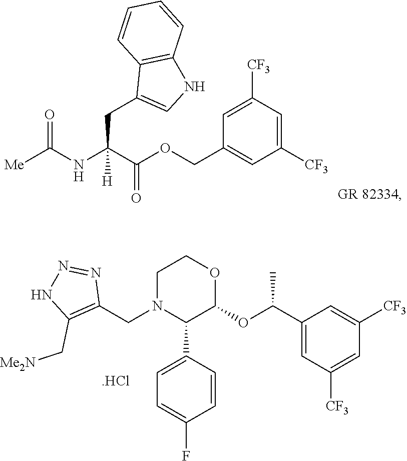
5-[[(2R,3S)-2-[(1R)-1-[3,5-Bis(trifluoromethyl)phenyl]ethoxy]-3-(4-fluorophenyl)-4-morpholinyl]methyl-N,N-dimethyl-1H-1,2,3-triazole-4-methanamine hydrochloride, 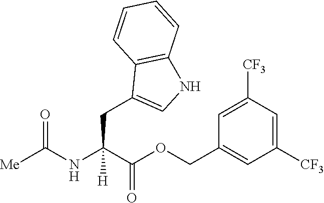
N-Acetyl-L-tryptophan 3,5-bis(trifluoromethyl)benzyl ester, 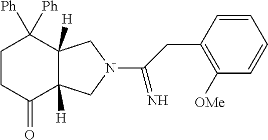 [SMILES: COc1ccccc1CC(=N)N1C[C@@H]2C(=O)CCC(c3ccccc3)(c3ccccc3)[C@@H]2C1]
(3aR;7aR)-Octahydro-2-[1-imino-2-(2methoxyphenyl)ethyl]-7,7-diphenyl-4H-isoindol, 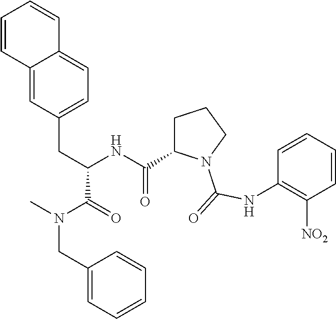 [SMILES: CN(Cc1ccccc1)C(=O)[C@H](Cc1ccc2ccccc2c1)NC(=O)[C@@H]1CCCN1C(=O)Nc1ccccc1[N+](=O)[O-]]
1-[[(2-Nitrophenyl)amino]carbonyl]-L-prolyl-N-methyl-3-(2-naphthalenyl)-N-(phenylmethyl)-L-alaninamide; 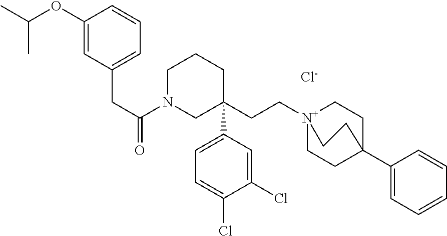 [SMILES: CC(C)Oc1cccc(CC(=O)N2CCC[C@](CC[N+]34CCC(c5ccccc5)(CC3)CC4)(c3ccc(Cl)c(Cl)c3)C2)c1 ; [Cl-]]
1-[2-[(3S)-3-(3,4-Dichlorophenyl)-1-[2-[3-(1-methylethoxy)phenyl]acetyl]-3-piperidinyl]ethyl]-4-phenyl-1-azoniabicyclo[2.2.2]octane chloride, analogs or combinations thereof; or said nucleic acid is one selected from an aptamer, a small interfering RNA, a microRNA, a small hairpin RNA and an antisense nucleic acid.

27. (canceled)

28. The method of claim 23, wherein the composition is administered to said subject by a topical administration, a subconjunctival administration, an intravitreal administration, or an ocular administration.

29. The method of claim 28, wherein the composition is topically administered to said subject at least once a day, at least twice a day, or at least three times a day.

30. (canceled)

31. (canceled)

32. (canceled)

33. The method of claim 23, wherein said composition is administered to said subject in combination with a secondary therapy or a secondary agent.

34. A neurokinin 1 receptor (NK1R) antagonist comprising a compound having a formula (I), 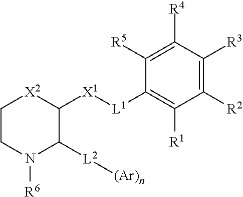
(I), or a pharmaceutically acceptable salt thereof;
wherein:
Ar is substituted or unsubstituted aryl or heteroaryl,
n is an integer from 1 to 3,
X¹ is —NH—, —C(O)— or —O—,
X² is —CHR⁷— or —O—,
L¹ is a bond, or substituted or unsubstituted C₁-C₄ alkylene,
L² is a bond, or substituted or unsubstituted C₁-C₄ alkylene,
each R¹, R², R³, R⁴, R⁵, R⁶, and R⁷ is independently hydrogen, halogen, substituted or unsubstituted C₁-C₄ alkylene, substituted or unsubstituted 2 to 4 membered heteroalkyl, substituted or unsubstituted cycloalkyl, substituted or unsubstituted heterocycloalkyl, substituted or unsubstituted aryl, or substituted or unsubstituted heteroaryl; or R⁶ and R⁷ are jointed to form a substituted or unsubstituted heterocycloalkyl.

35. (canceled)

36. (canceled)

37. (canceled)

38. (canceled)

39. The neurokinin 1 receptor (NK1R) antagonist of claim 34, wherein the compound has the following formula, 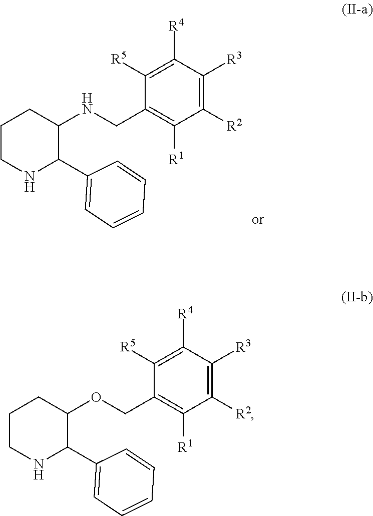
or a pharmaceutically acceptable salt thereof.

40. (canceled)

41. The NK1R antagonist of claim 39, wherein R³ is hydrogen; R¹ or R⁵ is independently hydrogen or —OCH₃; and/or R² or R⁴ is independently hydrogen, 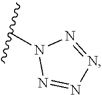 [SMILES: *n1nnnn1]
—CF₃ or —OCF₃.

42. (canceled)

43. (canceled)

44. The NK1R antagonist of claim 39, wherein the compound comprises:

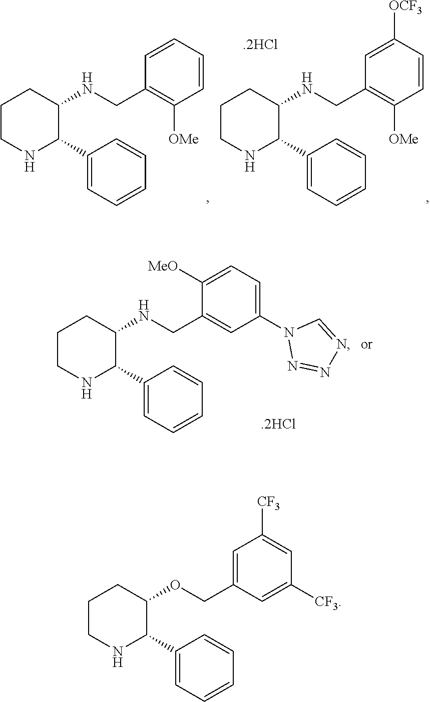

45. (canceled)

46. (canceled)

47. (canceled)

48. (canceled)

49. The neurokinin 1 receptor (NK1R) antagonist of claim 34, wherein the antagonist is a compound having a formula (III-a). 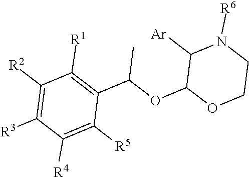
(III-a), or a pharmaceutically acceptable salt thereof.

50. The NK1R antagonist of claim 49, wherein Ar is substituted or unsubstituted phenyl; R⁶ is substituted C₁-C₄ alkyl,

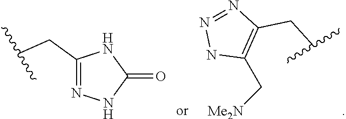 [SMILES: *Cc1n[nH]c(=O)[nH]1 ; *Cc1nn[nH]c1CN(C)C]

51. (canceled)

52. (canceled)

53. (canceled)

54. (canceled)

55. The NK1R antagonist of claim 49, wherein R³ is hydrogen; each R¹ and R⁵ is independently hydrogen; and/or each R² and R⁴ is independently hydrogen or —CF₃.

56. (canceled)

57. (canceled)

58. The NK1R antagonist of claim 49, wherein the compound comprises

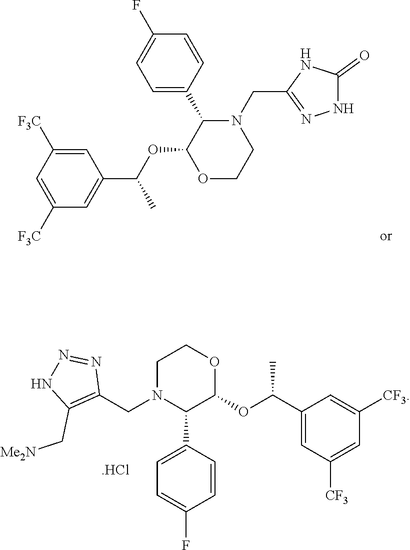 [SMILES: C[C@@H](O[C@H]1OCCN(Cc2n[nH]c(=O)[nH]2)[C@H]1c1ccc(F)cc1)c1cc(C(F)(F)F)cc(C(F)(F)F)c1 ; C[C@@H](O[C@H]1OCCN(Cc2nn[nH]c2CN(C)C)[C@H]1c1ccc(F)cc1)c1cc(C(F)(F)F)cc(C(F)(F)F)c1 ; ".HCl"]

59. (canceled)

60. (canceled)

61. (canceled)

62. The neurokinin 1 receptor (NK1R) antagonist of claim 61, wherein the antagonist is a compound having a formula (IV-a), 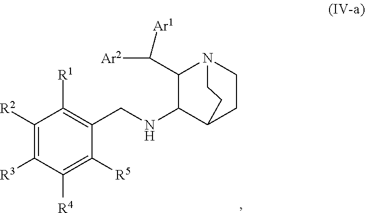
or a pharmaceutically acceptable salt thereof
wherein the Ar¹ and Ar² are phenyl; each R¹ and R⁵ is independently hydrogen, or —OCH₃; and/or R², R³ and R⁴ are hydrogen.

63. (canceled)

64. (canceled)

65. (canceled)

66. The NK1R antagonist of claim 62, wherein each compound of formula (IV) includes

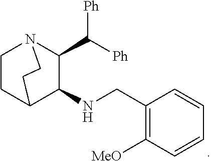 [SMILES: COc1ccccc1CN[C@H]1C2CCN(CC2)[C@H]1C(c1ccccc1)c1ccccc1]

67. A topical ocular formulation comprising a neurokinin 1 receptor (NK1R) antagonist of claim 34.

68. A pharmaceutical composition comprising a neurokinin 1 receptor (NK1R) antagonist of claim 34.


SEQUENCE LISTING (4 sequences)

SEQ ID NO: 1 - protein, 407 aa, Homo sapiens
MDNVLPVDSDLSPNISTNTSEPNQFVQPAWQIVLWAAAYTVIVVTSVVGNVVVMWIILAHKRMRTVTNYFLVNLAFAEASMAAFNTVVNFTYAVHNEWYYGLFYCKFHNFFPIAAVFASIYSMTAVAFDRYMAIIHPLQPRLSATATKVVICVIWVLALLLAFPQGYYSTTETMPSRVVCMIEWPEHPNKIYEKVYHICVTVLIYFLPLLVIGYAYTVVGITLWASEIPGDSSDRYHEQVSAKRKVVKMMIVVVCTFAICWLPFHIFFLLPYINPDLYLKKFIQQVYLAIMWLAMSSTMYNPIIYCCLNDRFRLGFKHAFRCCPFISAGDYEGLEMKSTRYLQTQGSVYKVSRLETTISTVVGAHEEEPEDGPKATPSSLDLTSNCSSRSDSKTMTESFSFSSNVLS

SEQ ID NO: 2 - protein, 11 aa, Artificial Sequence
  Description of Artificial Sequence Synthetic peptide
RPKPQQWFWLL

SEQ ID NO: 3 - protein, 6 aa, Artificial Sequence
  Description of Artificial Sequence Synthetic peptide
LDQWFG

SEQ ID NO: 4 - protein, 6 aa, Artificial Sequence
  Description of Artificial Sequence Synthetic peptide
QWFGLM